(19) United States
(12) Patent Application Publication
(10) Pub. No.: US 2024/0262803 A1
(43) Pub. Date: Aug. 8, 2024

(54) COMPOUNDS, COMPOSITIONS AND METHODS OF TREATING DISORDERS

(71) Applicant: Chungdu Anticancer Bioscience Ltd, Chengdu (CN)

(72) Inventors: Dun Yang, Chengdu (CN); Jing Zhang, Chengdu (CN); Shenqiu Zhang, St. Andrews (GB); Thaddeus Allen, Daly City, CA (US); Qiong Shi, Chengdu (CN); Gang Lv, Chengdu (CN); Hongmei Li, Chengdu (CN); Chenglu Yang, Chengdu (CN)

(21) Appl. No.: 18/283,539

(22) PCT Filed: Mar. 24, 2022
(86) PCT No.: PCT/CN2022/082691

§ 371 Date: Sep. 22, 2023

(30) Foreign Application Priority Data
Mar. 24, 2021 (WO) PCT/CN2021/082684

(51) Int. Cl. C07D 401/04; A61K 31/444; A61K 31/497; A61K 31/506; A61P 35/00; C07D 213/647; C07D 231/12; C07D 241/18; C07D 401/14; C07D 405/14; C07D 413/14

(52) U.S. Cl. CPC C07D 401/04; A61K 31/444; A61K 31/497; A61K 31/506; A61P 35/00; C07D 213/647; C07D 231/12; C07D 241/18; C07D 401/14; C07D 405/14; C07D 413/14

(57) ABSTRACT

The present disclose includes, among other things, compounds that treat or lessen the severity of cancer, pharmaceutical compositions and methods of making and using the same.

### BACKGROUND

Cancer is a term used for diseases in which abnormal cells divide without control and may invade other tissues. Cancer cells may also spread to other parts of the body through the blood and lymph systems.

There are more than 100 different types of cancer, with most cancers named for the organ or type of cell in which they start. For example, cancer that begins in the colon may be referred to as colon cancer; cancer that begins in basal cells of the skin may be referred to as basal cell carcinoma. Common types of cancer include breast cancer and lung cancer.

Cancer types can also be grouped into broader categories. The main categories of cancer include: carcinoma-cancer that begins in the skin or in tissues that line or cover internal organs; sarcoma—cancer that begins in bone, cartilage, fat, muscle, blood vessels, or other connective or supportive tissue; leukemia-cancer that starts in blood-forming tissue such as the bone marrow and causes large numbers of abnormal blood cells to be produced and enter the blood; lymphoma and myeloma—cancers that begin in the cells of the immune system; central nervous system cancers—cancers that begin in the tissues of the brain and spinal cord.

Several techniques for treating cancer are known in the art. Such techniques include chemotherapy, radiation therapy, surgery, and transplantation. Each of these techniques, however, have undesirable side effects and varying success rates. Therefore, a need exists to develop new methods for treating cancer and/or diseases associated with cellular proliferation.

### SUMMARY

The present disclosure provides for compounds of formula (I):

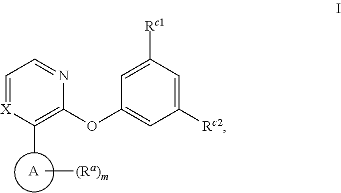

or a pharmaceutically acceptable salt thereof. Additionally, the present disclosure includes, among other things, pharmaceutical compositions, methods of using and methods of making a compound of formula (I).

### BRIEF DESCRIPTION OF DRAWINGS

FIG. 1 depicts minimal effective concentrations by which 86 compounds elicit polyploidy in the RPEMYCH2B-GFP cell line.

FIG. 2A depicts NCI-H23 cell lines and that compound #2 Suppresses the Anchorage-independent Growth of Cancer Cells in 3D Culture.

FIG. 2B shows that Compounds #1, #21 and #23 Suppress the Anchorage-independent Growth of Cancer Cells in 3D Culture.

FIG. 3 depicts a graph that shows compounds #9, #10 and #29 suppress the growth of human lung cancer cell line NCI-H23 in immunocompromised mice.

FIG. 4 depicts a graph that shows compounds #9, #10 and #29 suppress the growth of human lung cancer cell line HCT116 in immunocompromised mice.

### DETAILED DESCRIPTION

In some embodiments, the present disclosure includes a compound of Formula (I):

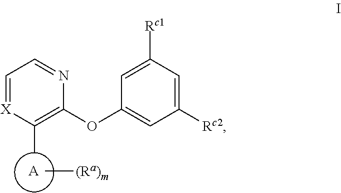

or a pharmaceutically acceptable salt,
   wherein
X is —CH═ or —N═;
Group A is optionally substituted phenyl or optionally substituted 6-membered heteroaryl; each R^{a} is independently selected from the group consisting of halogen, —CN, —OH, —OR¹, —SR¹, —NH₂, —NR²R³, —C(O)R¹, —C(O)OH, —C(O)OR¹, —C(O)NH₂, —C(O)NR²R³, optionally substituted C₁-C₇ aliphatic, optionally substituted phenyl, optionally substituted 5-10 membered heteroaryl, and optionally substituted 5-10 membered heterocyclyl;
R^{c1} is selected from the group consisting of C₁-C₆ haloalkyl, halogen, —OR¹, —C(O)OR¹, and optionally substituted 5-6 membered heteroaryl;
R^{c2} is selected from the group consisting of halogen, —CN, —OH, —OR¹, —NH₂, —NR²R³, optionally substituted C₁-C₇ aliphatic, optionally substituted phenyl, —C(O)R¹, —C(O)OH, —C(O)OR¹, —C(O)NH₂, —C(O)NR²R³, optionally substituted 5-10 membered heteroaryl, and optionally substituted 5-10 membered heterocyclyl;
each R¹ is independently selected from the group consisting of optionally substituted C₁-C₆ aliphatic, optionally substituted phenyl, optionally substituted 5-10 membered heteroaryl, and optionally substituted 5-10 membered heterocyclyl;
each R² is independently selected from the group consisting of —C(O)R¹, —C(O)OR¹, —C(O)NR¹R³, optionally substituted C₁-C₆ aliphatic, optionally substituted phenyl, optionally substituted 5-10 membered heteroaryl, and optionally substituted 5-10 membered heterocyclyl;
each R³ is independently selected from the group consisting of hydrogen, —C(O)R¹, —C(O)OR¹, —C(O)NHR¹, optionally substituted C₁-C₆ aliphatic, optionally substituted phenyl, optionally substituted 5-10 membered heteroaryl, and optionally substituted 5-10 membered heterocyclyl; and
m is 0, 1, 2, 3, 4, or 5.

In some embodiments, present disclosure includes a compound of formula (I-a):

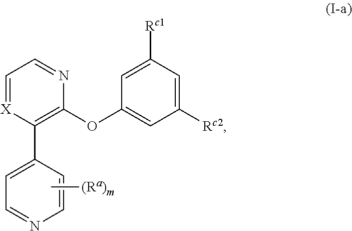

or a pharmaceutically acceptable salt thereof, wherein X, R^{a}, R^{c1}, R^{c2}, and m are defined above and described in classes and subclasses herein.

In some embodiments, present disclosure includes a compound of formula (I-b):

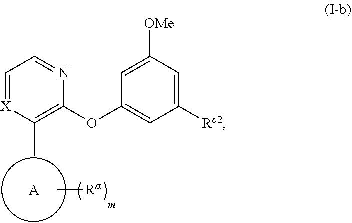

or a pharmaceutically acceptable salt thereof, wherein Ring A, X, R^{a}, R^{c2}, and m are defined above and described in classes and subclasses herein.

In some embodiments, present disclosure includes a compound of formula (I-c):

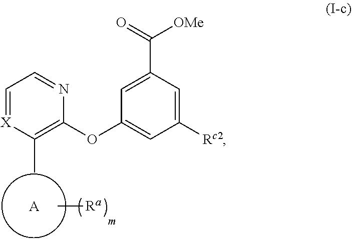

or a pharmaceutically acceptable salt thereof, wherein Ring A, X, R^{a}, R^{c2}, and m are defined above and described in classes and subclasses herein.

In some embodiments, present disclosure includes a compound of formula (I-d):

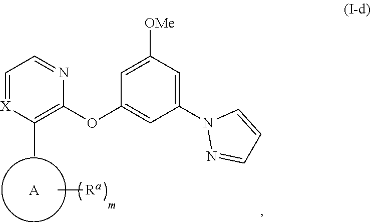

or a pharmaceutically acceptable salt thereof, wherein Ring A, X, R^{a}, and m are defined above and described in classes and subclasses herein.

In some embodiments, the present disclosure includes a compound of Formula (II):

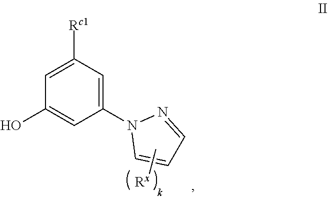

or a pharmaceutically acceptable salt,
wherein
R^{c1} is selected from the group consisting of C₁-C₆ haloalkyl, —OR¹, —C(O)OR¹, and optionally substituted 5-6 membered heteroaryl;
R^{x} is selected from the group consisting of halogen, —CN, —OH, —OR¹, —NH₂, —NR²R³, optionally substituted C₁-C₇ aliphatic, optionally substituted phenyl, —C(O)R¹, —C(O)OH, —C(O)OR¹, —C(O)NH₂, —C(O)NR²R³, optionally substituted 5-10 membered heteroaryl, and optionally substituted 5-10 membered heterocyclyl; and
k is 0, 1, 2, or 3.

In some embodiments, the present disclosure includes a compound of Formula (II-a):

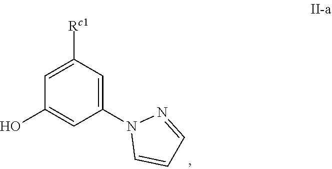

or a pharmaceutically acceptable salt thereof, wherein R^{c1} is defined above and described in classes and subclasses herein.

### Group A

In some embodiments, Group A is optionally substituted phenyl or optionally substituted 6-membered heteroaryl. In some embodiments, Group A is optionally substituted phenyl. In some embodiments, Group A is optionally substituted 6-membered heteroaryl. In some embodiments, Group A is optionally substituted pyridine or optionally substituted pyrimidine. In some embodiments, Group A is optionally substituted pyridine. In some embodiments, Group A is optionally substituted pyrimidine.

In some embodiments, Ring A is selected from the group consisting of

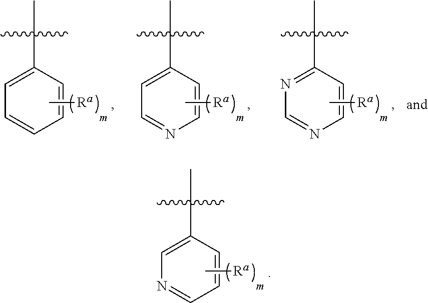

In some embodiments, Ring A is selected from the group consisting of

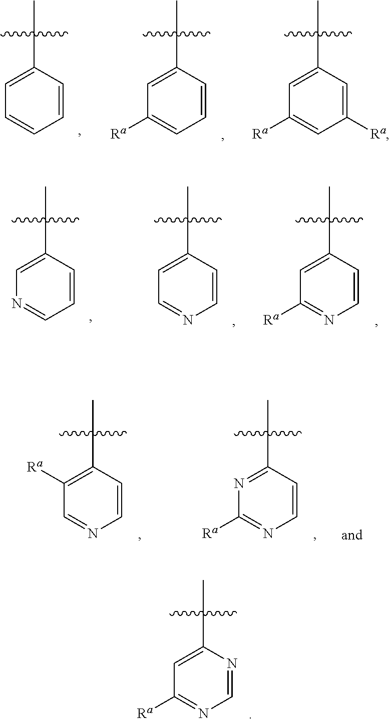

### X

In some embodiments, X is —CH═ or —N═. In some embodiments, X is —CH═. In some embodiments, X is —N═

### R^{a}

In some embodiments, each R is independently selected from the group consisting of halogen, —CN, —OH, —OR¹, —SR¹, —NH₂, —NR²R³, —C(O)R¹, —C(O)OH, —C(O)OR¹, —C(O)NH₂, —C(O)NR²R³, optionally substituted C₁-C₇ aliphatic, optionally substituted phenyl, optionally substituted 5-10 membered heteroaryl, and optionally substituted 5-10 membered heterocyclyl. In some embodiments, each R^{a} is independently selected from the group consisting of halogen, —NH₂, —NR²R³, —OR¹, —SR¹, and optionally substituted C₁-C₇ aliphatic. In some embodiments, each R^{a} is independently selected from the group consisting of —Cl, —F, —CF₃, -Me, —NH₂, —NHMe, OMe, —OCH₂CH₂OCH₂CH₃, and —SMe.

### R^{c1}

In some embodiments, R^{c1} is selected from the group consisting of C₁-C₆ haloalkyl, halogen, —OR¹, —C(O)OR¹, and optionally substituted 5-6 membered heteroaryl. In some embodiments, R^{c1} is selected from the group consisting of C₁-C₃ haloalkyl, halogen, —OR¹, —C(O)OR¹, and optionally substituted 5-membered heteroaryl. In some embodiments, R^{c1} is selected from the group consisting of —OR¹, —C(O)OR¹, and optionally substituted 5-membered heteroaryl. In some embodiments, R^{c1} is —OMe, —OEt, —C(O)OMe, —CF₃, —OCF₃, —Br, pyrazole, and oxadiazole.

In some embodiments, R^{c1} is —OR¹. In some embodiments, R^{c1} is —OMe or —OEt.

In some embodiments, R^{c1} is —C(O)OR¹. In some embodiments, R^{c1} is —C(O)OMe.

### R^{c2}

In some embodiments R^{c2} is selected from the group consisting of halogen, —CN, —OH, —OR¹, —NH₂, —NR²R³, optionally substituted C₁-C₇ aliphatic, optionally substituted phenyl, —C(O)R¹, —C(O)OH, —C(O)OR¹, —C(O)NH₂, —C(O)NR²R³, optionally substituted 5-10 membered heteroaryl, and optionally substituted 5-10 membered heterocyclyl. In some embodiments, R^{∘2} is selected from the group consisting of —C(O)NR²R³, optionally substituted pyrazole, optionally substituted oxadiazole, optionally substituted triazole, OR¹, —C(O)OR¹, —NR²R³, C₁-C₆, —C(O)OH, haloalkyl,

In some embodiments, R^{c2} is optionally substituted 5-membered heteroaryl. In some embodiments, R^{c2} is selected from the group consisting of optionally substituted pyrazole, optionally substituted oxadiazole, and optionally substituted triazole.

### R¹

In some embodiments, each R¹ is independently selected from the group consisting of optionally substituted C₁-C₆ aliphatic, optionally substituted phenyl, optionally substituted 5-10 membered heteroaryl, and optionally substituted 5-10 membered heterocyclyl. In some embodiments, each R¹ is independently selected from the group consisting of optionally substituted C₁-C₆ aliphatic. In some embodiments, each R¹ is independently selected from the group consisting of -Me, -Et or —CF₃.

### R²

In some embodiments, each R² is independently selected from the group consisting of —C(O)R¹, —C(O)OR¹, —C(O)NR¹R³, optionally substituted C₁-C₆ aliphatic, optionally substituted phenyl, optionally substituted 5-10 membered heteroaryl, and optionally substituted 5-10 membered heterocyclyl. In some embodiments, each R² is independently optionally substituted C₁-C₆ aliphatic. In some embodiments, R² is optionally substituted methyl.

### R³

In some embodiments, each R³ is independently selected from the group consisting of hydrogen, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl. In some embodiments, R³ is hydrogen. In some embodiments, each R³ is independently optionally substituted C₁-C₆ aliphatic. In some embodiments, each R³ is independently selected from the group consisting of hydrogen, optionally substituted methyl, —CF₃, and —CHF₂.

In some embodiments, the present disclosure includes compounds described in Table 1.

TABLE 1

| NO. | Structure |
|---|---|
| 1 | 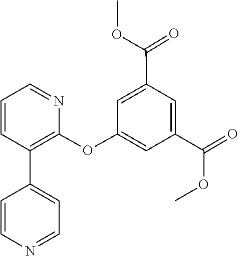 [SMILES: COC(=O)c1cc(Oc2ncccc2-c2ccncc2)cc(C(=O)OC)c1] |
| 2 | 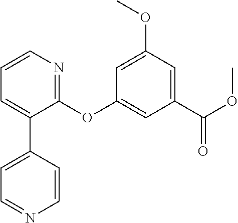 [SMILES: COC(=O)c1cc(OC)cc(Oc2ncccc2-c2ccncc2)c1] |
| 3 | 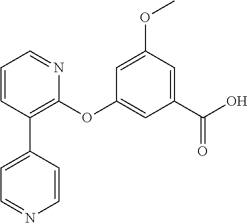 [SMILES: COc1cc(Oc2ncccc2-c2ccncc2)cc(C(=O)O)c1] |
| 4 | 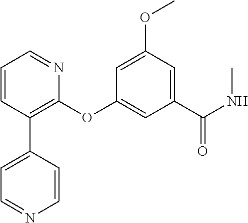 [SMILES: CNC(=O)c1cc(OC)cc(Oc2ncccc2-c2ccncc2)c1] |
| 5 | 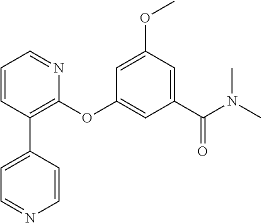 [SMILES: COc1cc(Oc2ncccc2-c2ccncc2)cc(C(=O)N(C)C)c1] |
| 6 | 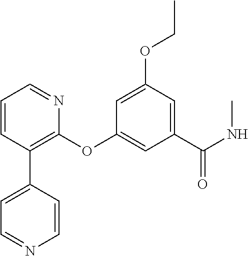 [SMILES: CCOc1cc(Oc2ncccc2-c2ccncc2)cc(C(=O)NC)c1] |
| 7 | 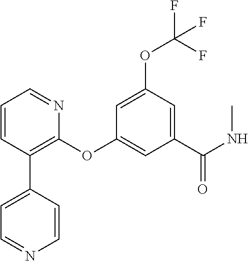 [SMILES: CNC(=O)c1cc(Oc2ncccc2-c2ccncc2)cc(OC(F)(F)F)c1] |
| 8 | 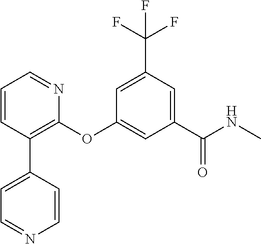 [SMILES: CNC(=O)c1cc(Oc2ncccc2-c2ccncc2)cc(C(F)(F)F)c1] |
| 9 | 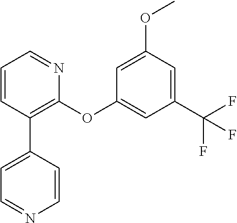 [SMILES: COc1cc(Oc2ncccc2-c2ccncc2)cc(C(F)(F)F)c1] |
| 10 | 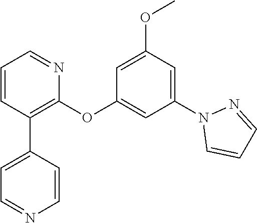 [SMILES: COc1cc(Oc2ncccc2-c2ccncc2)cc(-n2cccn2)c1] |
| 11 | 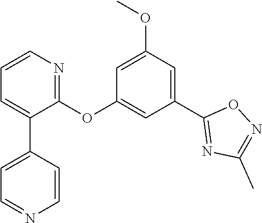 [SMILES: COc1cc(Oc2ncccc2-c2ccncc2)cc(-c2nc(C)no2)c1] |
| 12 | 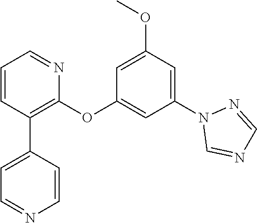 [SMILES: COc1cc(Oc2ncccc2-c2ccncc2)cc(-n2cncn2)c1] |
| 13 | 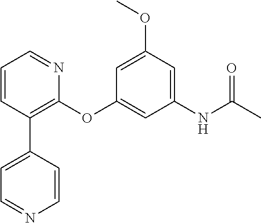 [SMILES: COc1cc(NC(C)=O)cc(Oc2ncccc2-c2ccncc2)c1] |
| 14 | 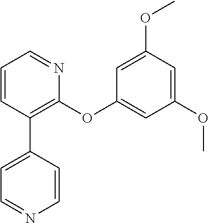 [SMILES: COc1cc(OC)cc(Oc2ncccc2-c2ccncc2)c1] |
| 15 | 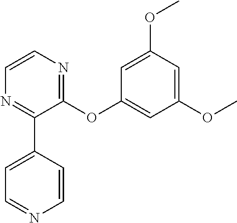 [SMILES: COc1cc(OC)cc(Oc2nccnc2-c2ccncc2)c1] |
| 17 | 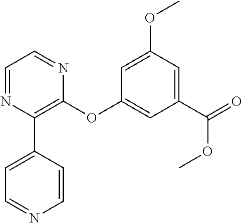 [SMILES: COC(=O)c1cc(OC)cc(Oc2nccnc2-c2ccncc2)c1] |
| 18 | 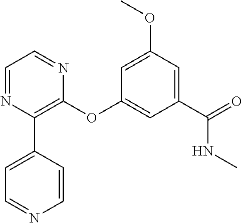 [SMILES: CNC(=O)c1cc(OC)cc(Oc2nccnc2-c2ccncc2)c1] |
| 19 | 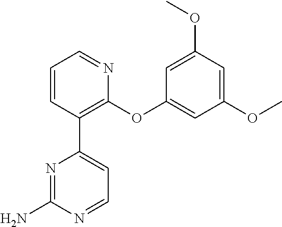 [SMILES: COc1cc(OC)cc(Oc2ncccc2-c2ccnc(N)n2)c1] |
| 21 | 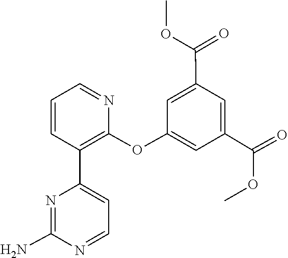 [SMILES: COC(=O)c1cc(Oc2ncccc2-c2ccnc(N)n2)cc(C(=O)OC)c1] |
| 22 | 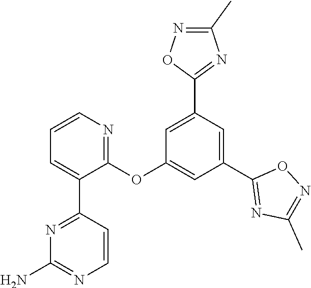 [SMILES: Cc1noc(-c2cc(Oc3ncccc3-c3ccnc(N)n3)cc(-c3nc(C)no3)c2)n1] |
| 23 | 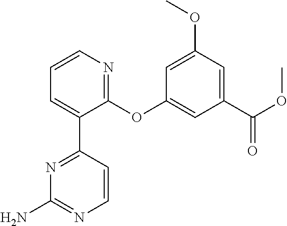 [SMILES: COC(=O)c1cc(OC)cc(Oc2ncccc2-c2ccnc(N)n2)c1] |
| 24 | 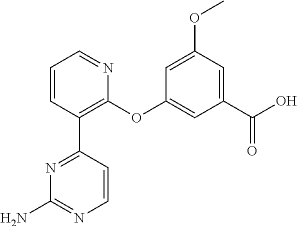 [SMILES: COc1cc(Oc2ncccc2-c2ccnc(N)n2)cc(C(=O)O)c1] |
| 25 | 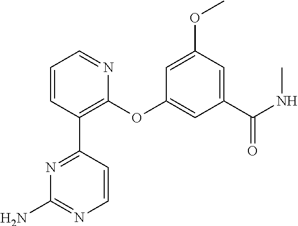 [SMILES: CNC(=O)c1cc(OC)cc(Oc2ncccc2-c2ccnc(N)n2)c1] |
| 26 | 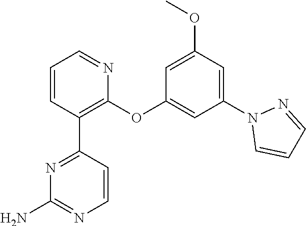 [SMILES: COc1cc(Oc2ncccc2-c2ccnc(N)n2)cc(-n2cccn2)c1] |
| 27 | 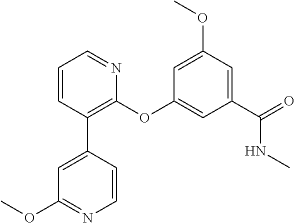 |
| 28 | 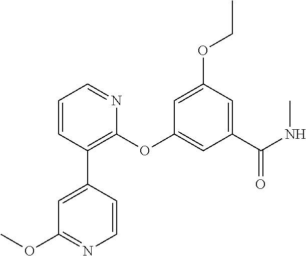 |
| 29 | 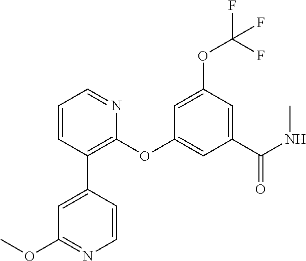 [SMILES: CNC(=O)c1cc(Oc2ncccc2-c2ccnc(OC)c2)cc(OC(F)(F)F)c1] |
| 30 | 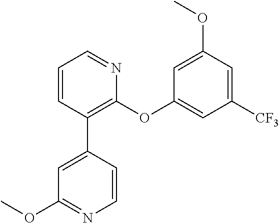 [SMILES: COc1cc(Oc2ncccc2-c2ccnc(OC)c2)cc(C(F)(F)F)c1] |
| 31 | 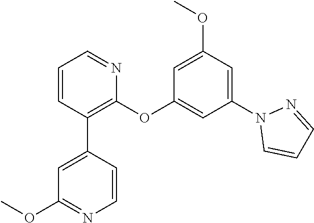 [SMILES: COc1cc(Oc2ncccc2-c2ccnc(OC)c2)cc(-n2cccn2)c1] |
| 32 | 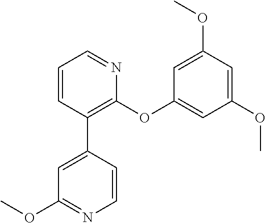 |
| 34 | 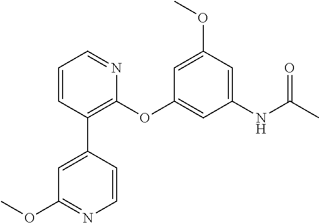 [SMILES: COc1cc(NC(C)=O)cc(Oc2ncccc2-c2ccnc(OC)c2)c1] |
| 35 | 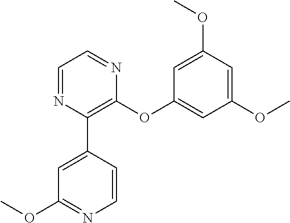 |
| 36 | 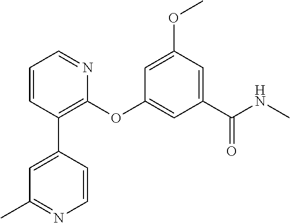 |
| 37 | 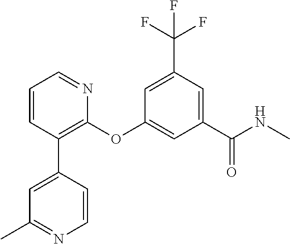 |
| 38 | 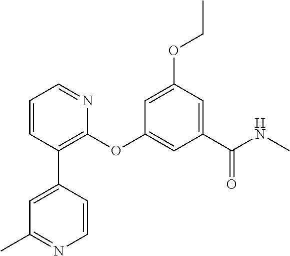 |
| 39 | 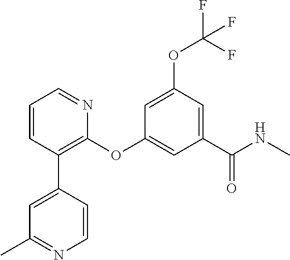 |
| 40 | 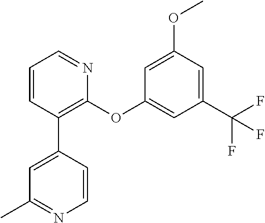 [SMILES: COc1cc(Oc2ncccc2-c2ccnc(C)c2)cc(C(F)(F)F)c1] |
| 41 | 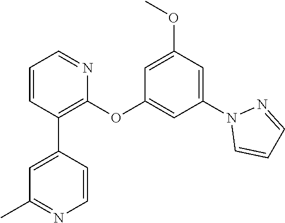 [SMILES: COc1cc(Oc2ncccc2-c2ccnc(C)c2)cc(-n2cccn2)c1] |
| 42 | 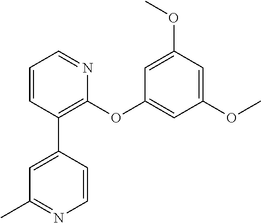 |
| 43 | 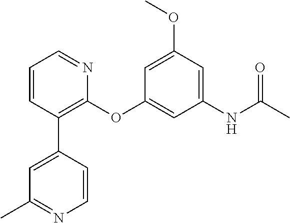 [SMILES: COc1cc(NC(C)=O)cc(Oc2ncccc2-c2ccnc(C)c2)c1] |
| 44 | 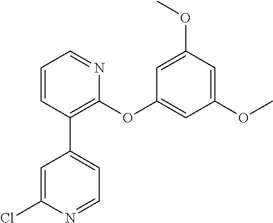 [SMILES: COc1cc(OC)cc(Oc2ncccc2-c2ccnc(Cl)c2)c1] |
| 45 | 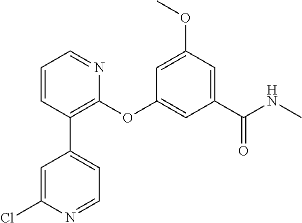 [SMILES: CNC(=O)c1cc(OC)cc(Oc2ncccc2-c2ccnc(Cl)c2)c1] |
| 46 | 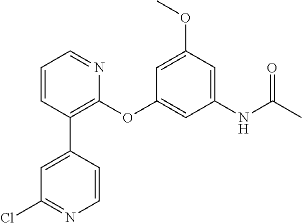 [SMILES: COc1cc(NC(C)=O)cc(Oc2ncccc2-c2ccnc(Cl)c2)c1] |
| 47 | 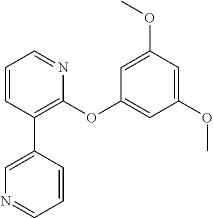 [SMILES: COc1cc(OC)cc(Oc2ncccc2-c2cccnc2)c1] |
| 50 | 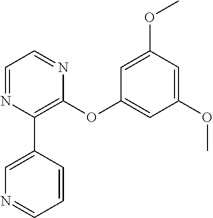 [SMILES: COc1cc(OC)cc(Oc2nccnc2-c2cccnc2)c1] |
| 52 | 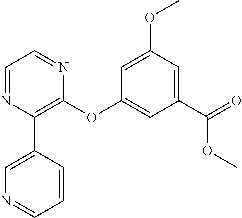 [SMILES: COC(=O)c1cc(OC)cc(Oc2nccnc2-c2cccnc2)c1] |
| 55 | 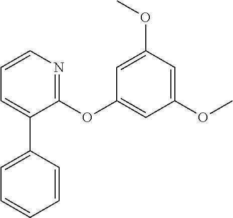 [SMILES: COc1cc(OC)cc(Oc2ncccc2-c2ccccc2)c1] |
| 56 | 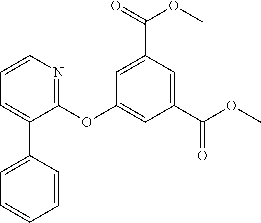 [SMILES: COC(=O)c1cc(Oc2ncccc2-c2ccccc2)cc(C(=O)OC)c1] |
| 57 | 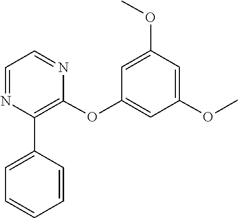 [SMILES: COc1cc(OC)cc(Oc2nccnc2-c2ccccc2)c1] |
| 58 | 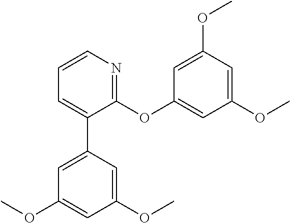 |
| 59 | 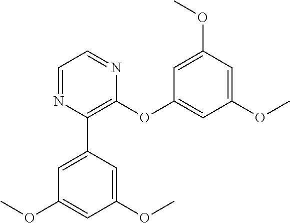 |
| 60 | 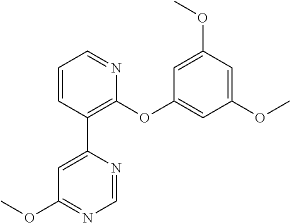 |
| 61 | 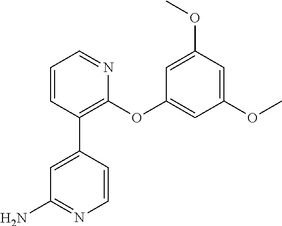 [SMILES: COc1cc(OC)cc(Oc2ncccc2-c2ccnc(N)c2)c1] |
| 62 | 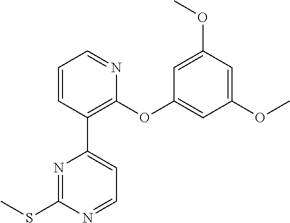 |
| 63 | 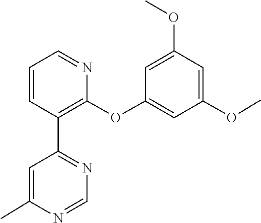 |
| 64 | 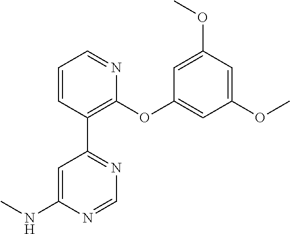 |
| 65 | 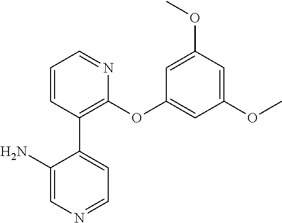 [SMILES: COc1cc(OC)cc(Oc2ncccc2-c2ccncc2N)c1] |
| 66 | 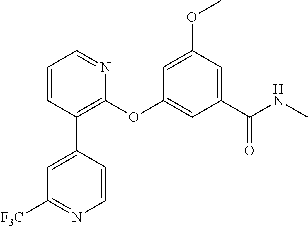 [SMILES: CNC(=O)c1cc(OC)cc(Oc2ncccc2-c2ccnc(C(F)(F)F)c2)c1] |
| 67 | 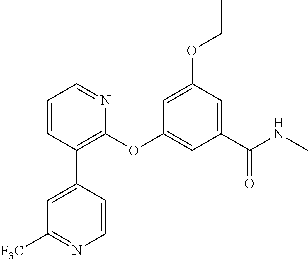 [SMILES: CCOc1cc(Oc2ncccc2-c2ccnc(C(F)(F)F)c2)cc(C(=O)NC)c1] |
| 81 | 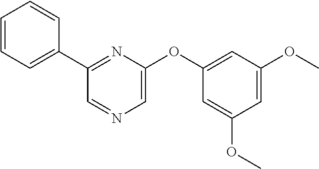 [SMILES: COc1cc(OC)cc(Oc2cncc(-c3ccccc3)n2)c1] |
| 82 | 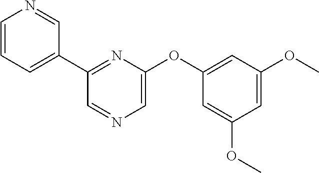 [SMILES: COc1cc(OC)cc(Oc2cncc(-c3cccnc3)n2)c1] |
| 83 | 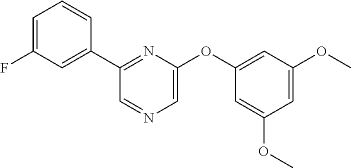 [SMILES: COc1cc(OC)cc(Oc2cncc(-c3cccc(F)c3)n2)c1] |
| 84 | 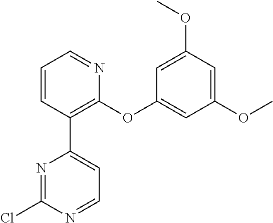 [SMILES: COc1cc(OC)cc(Oc2ncccc2-c2ccnc(Cl)n2)c1] |
| 85 | 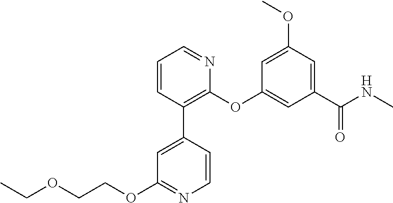 [SMILES: CCOCCOc1cc(-c2cccnc2Oc2cc(OC)cc(C(=O)NC)c2)ccn1] |
| 86 | 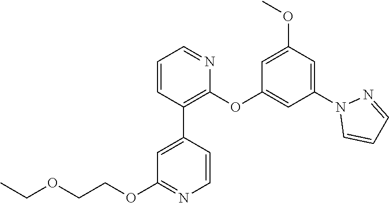 [SMILES: CCOCCOc1cc(-c2cccnc2Oc2cc(OC)cc(-n3cccn3)c2)ccn1] |
| 87 | 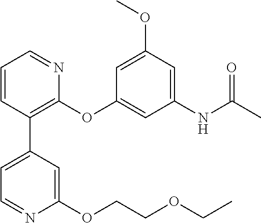 [SMILES: CCOCCOc1cc(-c2cccnc2Oc2cc(NC(C)=O)cc(OC)c2)ccn1] |
| 88 | 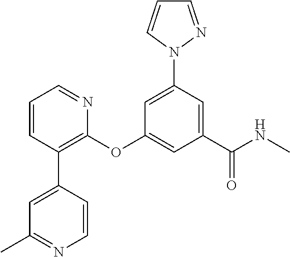 |
| 89 | 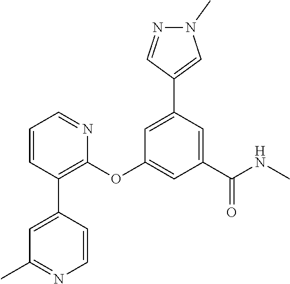 |
| 90 | 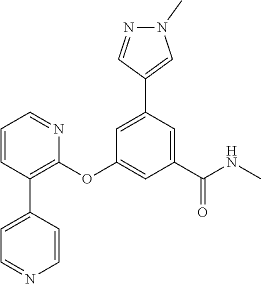 [SMILES: CNC(=O)c1cc(Oc2ncccc2-c2ccncc2)cc(-c2cnn(C)c2)c1] |
| 91 | 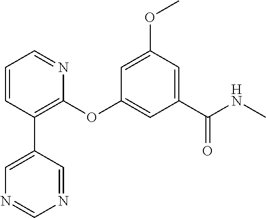 [SMILES: CNC(=O)c1cc(OC)cc(Oc2ncccc2-c2cncnc2)c1] |
| 92 | 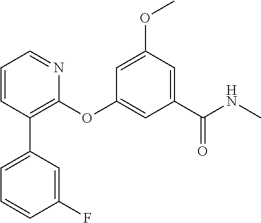 [SMILES: CNC(=O)c1cc(OC)cc(Oc2ncccc2-c2cccc(F)c2)c1] |
| 93 | 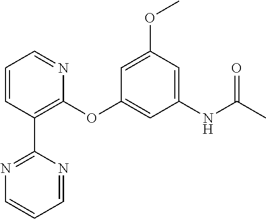 [SMILES: COc1cc(NC(C)=O)cc(Oc2ncccc2-c2ncccn2)c1] |
| 94 | 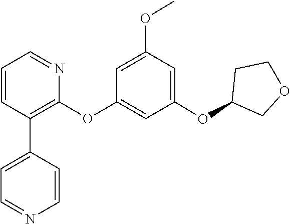 [SMILES: COc1cc(Oc2ncccc2-c2ccncc2)cc(O[C@H]2CCOC2)c1] |
| 95 | 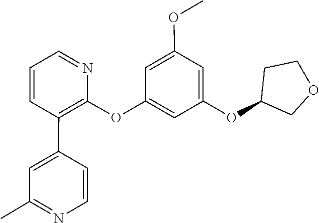 [SMILES: COc1cc(Oc2ncccc2-c2ccnc(C)c2)cc(O[C@H]2CCOC2)c1] |
| 96 | 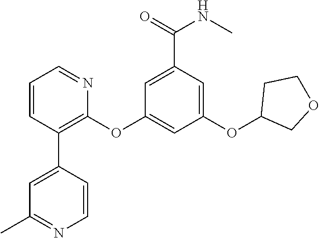 [SMILES: CNC(=O)c1cc(Oc2ncccc2-c2ccnc(C)c2)cc(OC2CCOC2)c1] |
| 97 | 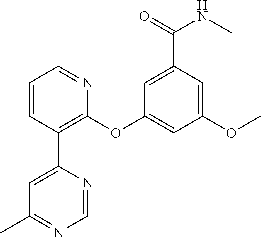 |
| 98 | 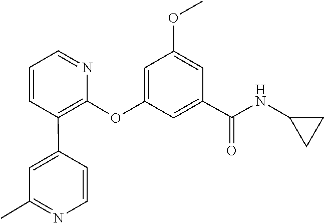 [SMILES: COc1cc(Oc2ncccc2-c2ccnc(C)c2)cc(C(=O)NC2CC2)c1] |
| 99 | 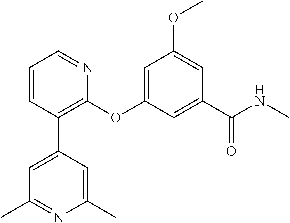 |
| 100 | 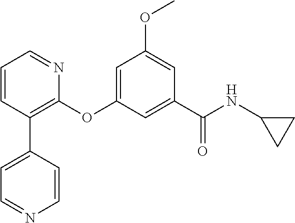 [SMILES: COc1cc(Oc2ncccc2-c2ccncc2)cc(C(=O)NC2CC2)c1] |
| 101 | 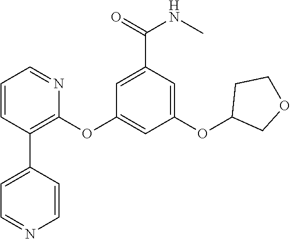 [SMILES: CNC(=O)c1cc(Oc2ncccc2-c2ccncc2)cc(OC2CCOC2)c1] |

### Definitions

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

The term “haloaliphatic” refers to an aliphatic group that is substituted with one or more halogen atoms.

The term “alkyl” refers to a straight or branched alkyl group. Exemplary alkyl groups are methyl, ethyl, propyl, isopropyl, butyl, isobutyl, and tert-butyl.

The term “haloalkyl” refers to a straight or branched alkyl group that is substituted with one or more halogen atoms.

The term “halogen” means F, Cl, Br, or I.

The term “aryl” used alone or as part of a larger moiety as in “aralkyl”, “aralkoxy”, or “aryloxyalkyl”, refers to monocyclic and bicyclic ring systems having a total of five to fourteen ring members, wherein at least one ring in the system is aromatic and wherein each ring in the system contains three to seven ring members. The term “aryl” may be used interchangeably with the term “aryl ring”. In certain embodiments of the present disclosure, “aryl” refers to an aromatic ring system which includes, but not limited to, phenyl, biphenyl, naphthyl, anthracyl and the like, which may bear one or more substituents. Also included within the scope of the term “aryl”, as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-”, used alone or as part of a larger moiety, e.g., “heteroaralkyl”, or “heteroaralkoxy”, refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring”, “heteroaryl group”, or “heteroaromatic”, any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle”, “heterocyclyl”, “heterocyclic radical”, and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in TV-substituted pyrrolidinyl). A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle”, “heterocyclyl”, “heterocyclyl ring”, “heterocyclic group”, “heterocyclic moiety”, and “heterocyclic radical”, are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable c m of an “optionally substituted” group are independently halogen; —(CH₂)₀₋₄R^{∘}; (CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; N(R^{∘})C(S)NR^{∘}₂; (CH₂)₀₋₄N(R^{∘})C(O)OR^{∘}; N(R^{∘})N(R^{∘})C(O)R^{∘}; N(R^{∘})N(R^{∘})C(O)NR^{∘}₂; —N(R^{∘})N(R^{∘})C(O)OR^{∘}; —(CH₂)₀₋₄C(O)R^{∘}; —C(S)R; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}, SC(S)SR^{∘}; (CH₂)₀₋₄SC(O)R^{∘}; (CH₂)₀₋₄C(O)NR^{∘}₂; C(S)NR^{∘}₂; C(S)SR^{∘}; SC(S)SR^{∘}, (CH₂)₀₋₄OC(O)NR^{∘}₂; C(O)N(OR^{∘})R^{∘}; C(O)C(O)R; C(O)CH₂C(O)R^{∘}; C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}2; —N(R^{∘})S(O)₂R; —N(OR^{∘})R^{∘}; —C(NH)NR^{∘}₂; P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, CH₂Ph, O(CH₂)₀₋₁Ph, CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, (CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘0} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R are independently halogen, R^{●}, -(haloR^{●}), —OH, —OR^{●}, —O(haloR^{●}), —CN, —C(O)OH, —C(O)OR^{●}, —NH₂, —NHR, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Combinations of substituents and variables envisioned by this disclosure are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylactic administration to a subject).

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof.

The term “biological sample”, as used herein, includes, without limitation, cell cultures or extracts thereof; biopsied material obtained from a mammal or extracts thereof; and blood, saliva, urine, feces, semen, tears, or other body fluids or extracts thereof. Examples of such purposes include, but are not limited to, blood transfusion, organ transplantation, biological specimen storage, and biological assays.

As used herein, a “therapeutically effective amount” means an amount of a substance (e.g., a therapeutic agent, composition, and/or formulation) that elicits a desired biological response. In some embodiments, a therapeutically effective amount of a substance is an amount that is sufficient, when administered as part of a dosing regimen to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of a provided compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition.

### I

As used herein, the terms “treatment,” “treat,” and “treating” refer to partially or completely alleviating, inhibiting, delaying onset of, preventing, ameliorating and/or relieving a disorder or condition, or one or more symptoms of the disorder or condition, as described herein. In some embodiments, treatment may be administered after one or more symptoms have developed. In some embodiments, the term “treating” includes preventing or halting the progression of a disease or disorder. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence. Thus, in some embodiments, the term “treating” includes preventing relapse or recurrence of a disease or disorder.

The term “patient”, as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound(s) with which it is formulated. Pharmaceutically acceptable carriers, adjuvants or vehicles that may be used in the compositions of the compounds disclosed herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” mneans any non-toxic salt, ester, salt of an ester or other derivative of a compound of this disclosure that, upon administration to a recipient, is capable of providing, either directly or indirectly, a compound of this disclosure or an inhibitorily active metabolite or residue thereof.

The expression “dosage unit form” as used herein refers to a physically discrete unit of agent appropriate for the patient to be treated. It will be understood, however, that total daily usage of compounds and compositions of the present disclosure will be decided by the attending physician within the scope of sound medical judgment. Specific effective dose level for any particular patient or organism will depend upon a variety of factors including disorder being treated and severity of the disorder; activity of specific compound employed; specific composition employed; age, body weight, general health, sex and diet of the patient; time of administration, route of administration, and rate of excretion of a specific compound employed; duration of treatment; drugs used in combination or coincidental with a specific compound employed, and like factors well known in the medical arts.

### Alternative Embodiments

In an alternative embodiment, compounds described herein may also comprise one or more isotopic substitutions. For example, hydrogen may be ²H (D or deuterium) or ³H (T or tritium); carbon may be, for example, ¹³C or ¹⁴C; oxygen may be, for example, 180; nitrogen may be, for example, ¹⁵N, and the like. In other embodiments, a particular isotope (e.g., ³H, ¹³C, ¹⁴C, ¹⁸O, or ¹⁵N) can represent at least 1%, at least 5%, at least 10%, at least 15%, at least 20%, at least 25%, at least 30%, at least 35%, at least 40%, at least 45%, at least 50%, at least 60%, at least 65%, at least 70%, at least 75%, at least 80%, at least 85%, at least 90%, at least 95%, at least 99%, or at least 99.9% of the total isotopic abundance of an element that occupies a specific site of the compound.

### Pharmaceutical Compositions

In some embodiments, the present disclosure provides a composition comprising a compound of Formula (I) and a pharmaceutically acceptable carrier, adjuvant, or vehicle. In some embodiments, the amount of compound in compositions contemplated herein is such that is effective to measurably treat a disease or disorder in a biological sample or in a patient. In certain embodiments, the amount of compound in compositions of this disclosure is such that is effective to measurably treat a disease or disorder in a biological sample or in a patient. In certain embodiments, a composition contemplated by this disclosure is formulated for administration to a patient in need of such composition. In some embodiments, a composition contemplated by this disclosure is formulated for oral administration to a patient.

In some embodiments, compositions of the present disclosure may be administered orally, parenterally, by inhalation spray, topically, rectally, nasally, buccally, vaginally or via an implanted reservoir. In some preferred embodiments, compositions are administered orally, intraperitoneally or intravenously. In some embodiments, sterile injectable forms of the compositions comprising one or more compounds of Formula (I) may be aqueous or oleaginous suspension. In some embodiments, suspensions may be formulated according to techniques known in the art using suitable dispersing or wetting agents and suspending agents. In some embodiments, sterile injectable preparation may also be a sterile injectable solution or suspension in a non-toxic parenterally acceptable diluent or solvent, for example as a solution in 1,3-butanediol. In some embodiments, among the acceptable vehicles and solvents that may be employed are water, Ringer's solution and isotonic sodium chloride solution. In some embodiments, additional examples include, but are not limited to, sterile, fixed oils are conventionally employed as a solvent or suspending medium.

The term “parenteral” as used herein includes subcutaneous, intravenous, intramuscular, intra-articular, intra-synovial, intrasternal, intrathecal, intrahepatic, intralesional and intracranial injection or infusion techniques.

Pharmaceutically acceptable compositions comprising one or more compounds of Formula (I) may be orally administered in any orally acceptable dosage form including, but not limited to, capsules, tablets, aqueous suspensions or solutions. In some embodiments, carriers used include lactose and corn starch. Lubricating agents, such as magnesium stearate, are also typically added. In some embodiments, useful diluents include lactose and dried cornstarch. In some embodiments, when aqueous suspensions are required for oral use, an active ingredient is combined with emulsifying and suspending agents. In some embodiments, certain sweetening, flavoring or coloring agents may also be added.

Alternatively, pharmaceutically acceptable compositions comprising a compound of Formula (I) may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions comprising a compound of Formula (I) may also be administered topically, especially when the target of treatment includes areas or organs readily accessible by topical application, including diseases of the eye, the skin, or the lower intestinal tract. Suitable topical formulations are readily prepared for each of these areas or organs. In some embodiments, pharmaceutically acceptable compositions may be formulated in a suitable ointment containing the active component suspended or dissolved in one or more carriers. Carriers for topical administration of compounds of this disclosure include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

Pharmaceutically acceptable compositions comprising a compound of Formula (I) may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

In some embodiments, an amount of a compound of the present disclosure that may be combined with the carrier materials to produce a composition in a single dosage form will vary depending upon the host treated, the particular mode of administration. Preferably, provided compositions should be formulated so that a dosage of between 0.01-100 mg/kg body weight/day of the inhibitor can be administered to a patient receiving these compositions.

### Methods of Using Compounds of the Present Disclosure

In some embodiments, the present disclosure provides a method for treating or lessening the severity of a disease or condition associated with cell proliferation in a patient comprising the step of administering to said patient a composition according to the present disclosure.

The term “disease or condition associated with cell proliferation”, as used herein means any disease or other deleterious condition in which cell proliferation is known to play a role. Accordingly, another embodiment of the present disclosure relates to treating or lessening the severity of one or more diseases in which cell proliferation is known to play a role. In some embodiments, a disease or condition associated with cell proliferation is cancer.

In some embodiments, administration of a compound of the present disclosure results in arrest of mitosis or change in DNA content.

In some embodiments, administration of a compound of the present disclosure results in arrest of mitosis. In some embodiments, mitotic arrest is defined as a 10-100% reduction in mitosis. In some embodiments, mitotic arrest is defined as a 20-100% reduction in mitosis. In some embodiments, mitotic arrest is defined as a 30-100% reduction in mitosis. In some embodiments, mitotic arrest is defined as a 40-100% reduction in mitosis. In some embodiments, mitotic arrest is defined as a 50-100% reduction in mitosis. In some embodiments, mitotic arrest is defined as a 60-100% reduction in mitosis. In some embodiments, mitotic arrest is defined as a 70-100% reduction in mitosis. In some embodiments, mitotic arrest is defined as a 80-100% reduction in mitosis. In some embodiments, mitotic arrest is defined as a 90-100% reduction in mitosis. In some embodiments, mitotic arrest is defined as a 100% reduction in mitosis.

In some embodiments, administration of a compound of the present disclosure results in change in DNA content. In some embodiments, change of DNA content is induction of polyploidy.

In some embodiments, compounds and compositions, according to a method of the present disclosure, may be administered using any amount and any route of administration effective for treating or lessening the severity of cancer. The exact amount required will vary from subject to subject, depending on the species, age, and general condition of the subject, severity of the infection, particular agent, its mode of administration, and the like. Compounds of the present disclosure are preferably formulated in dosage unit form for ease of administration and uniformity of dosage.

In some embodiments, cancer is selected from the group consisting of lung cancer and breast cancer. In some embodiments, cancer is lung cancer. In some embodiments, lung cancer is non-small cell lung cancer. In some embodiments, non-small cell lung cancer is lung adenocarcinoma. In some embodiments, cancer is breast cancer. In some embodiments, breast cancer is mammary cancer. In some embodiments, breast cancer is breast adenocarcinoma.

In some embodiments, pharmaceutically acceptable compositions of comprising compounds of the present disclosure can be administered to humans and other animals orally, rectally, parenterally, intracisternally, intravaginally, intraperitoneally, topically (as by powders, ointments, or drops), buccally, as an oral or nasal spray, or the like, depending on the severity of infection being treated. In certain embodiments, compounds of the present disclose may be administered orally or parenterally at dosage levels of about 0.01 mg/kg to about 50 mg/kg and preferably from about 1 mg/kg to about 25 mg/kg, of subject body weight per day, one or more times a day, to obtain desired therapeutic effect.

In some embodiments, one or more additional therapeutic agents, may also be administered in combination with compounds of the present disclosure. In some embodiments, a compound of the present disclosure and one or more additional therapeutic agents may be administered as part of a multiple dosage regime. In some embodiments, a compound of the present disclosure and one or more additional therapeutic agents may be administered may be administered simultaneously, sequentially or within a period of time. In some embodiments, a compound of the present disclosure and one or more additional therapeutic agents may be administered within five hours of one another. In some embodiments, a compound of the present disclosure and one or more additional therapeutic agents may be administered within 24 hours of one another. In some embodiments, a compound of the present disclosure and one or more additional therapeutic agents may be administered within one week of one another.

In some embodiments, a compound of the present disclosure and one or more additional therapeutic agents may be formulated into a single dosage form.

### Exemplification

### General Methods

Unless stated otherwise, all the chemicals required for synthesis were purchased from commercially available suppliers and used without further purification. ¹H NMR spectra was determined with a Bruker Avance III-400 at 400 MHz. LC-MS analysis was performed on a platform equipped with Agilent LC-MS 1260-6110 or Agilent LC-MS 1260-6120, using a Waters X Bridge C18: 50 mm×4.6 mm×3.5 um column. Flash column chromatography was conducted with silica gel (200-300 mesh, Qingdao Haiyang Chemical Co. Ltd., China). Analytical and preparative TLC analysis were performed on GF254 silica gel plates (Yantai Jiangyou Inc., China). Unless otherwise noted, reagents and all solvents are analytically pure grade and were obtained commercially from vendors such as Chron Chemical or Energy-Chemical.

Abbreviations: TLC: Thin Layer Chromatography, EA: Ethyl Acetate, PE: Petroleum Ether, DMF: N,N-dimethylformamide, THF: Eetrahydrofuran, DCM: Dichloromethane, DIPEA: N,N-diisopropylethylamine,

DMAP: 4-dimethylaminopyridine

### Synthesis of Intermediates

### Method A:

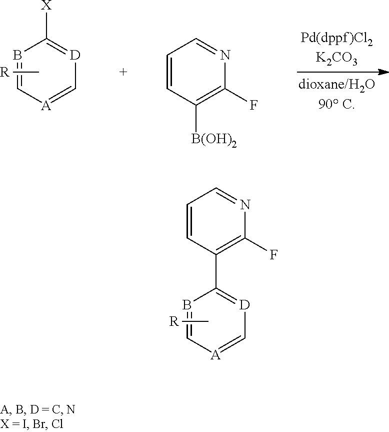

Aromatic halide (10 mmol, 1.0 eq), (2-fluoropyridin-3-yl)boronic acid (10 mmol, 1.0 eq), Pd(dppf)Cl₂ (182.7 mg, 0.25 mmol, 2.5% mol) and K₂CO₃ (4.5 g, 33 mmol, 3.3 eq) were added to a round-bottom flask with a magnetic bar, then dioxane (50 ml) and H₂O (6.25 ml) (v/v=8/1) were added as solvent. The reaction vessel was evacuated and backfilled with N₂ three times and protected with a balloon of N₂. The reaction mixture was heated at 90° C. for at least 5 h with vigorous stirring. The cooled solution was diluted with ethyl acetate (100 ml) and washed with brine (80 ml, 3 times). The organic phase was dried over anhydrous Na₂SO₄, and concentrated in vacuo. The residue was purified by silica gel flash chromatography to afford the product as a solid (yield=40%˜80%, purity>90%).

### Method B:

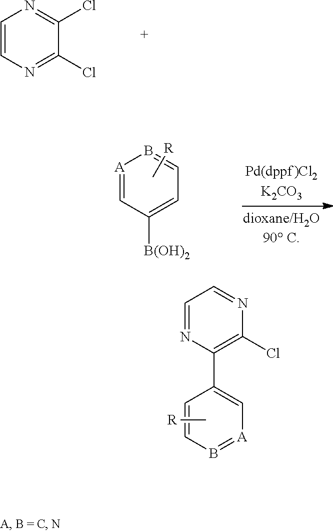

2,3-dichloropyrazine (10 mmol, 1.0 eq), organoboronic acid (10 mmol, 1.0 eq), Pd(dppf)Cl₂ (182.7 mg, 0.25 mmol, 2.5% mol) and K₂CO₃ (4.5 g, 33 mmol, 3.3 eq) were added to a round-bottom flask with a magnetic bar, then dioxane (50 ml) and H₂O (6.25 ml) (v/v=8/1) were added as solvent. The reaction vessel was evacuated and backfilled with N₂ three times and protected with a balloon of N₂. The reaction mixture was heated at 90° C. for at least 5 h with vigorous stirring. The cooled solution was diluted with ethyl acetate and washed with brine (80 ml, 3 times). The organic phase was dried over anhydrous Na₂SO₄, and concentrated in vacuo. The residue was purified by silica gel flash chromatography to afford the product as a solid (yield=40%˜60%, purity>90%).

### Method C:

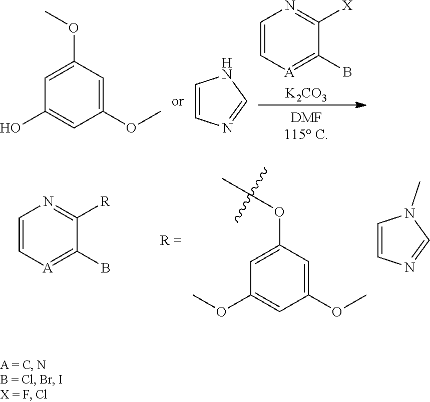

Halogenated aromatic compound (10 mmol, 1.0 eq), 3,5-dimethoxyphenol/1H-imidazole (10 mmol, 1.0 eq) and K₂CO₃ (2.76 g, 20 mmol, 2 eq) were added to a round-bottom flask with a magnetic bar, then DMF (50 ml) was added as solvent. The reaction vessel was evacuated and backfilled with N₂ three times and protected with a balloon of N₂. The reaction mixture was heated at 115° C. for at least 8 h with vigorous stirring. The cooled solution was diluted with ethyl acetate (100 ml) and washed with brine (80 ml, 3 times). The organic phase was dried over anhydrous Na₂SO₄, and concentrated in vacuo. The residue was purified by silica gel flash chromatography to afford the product as a white solid (yield=70%˜80%, purity>90%).

### Method D:

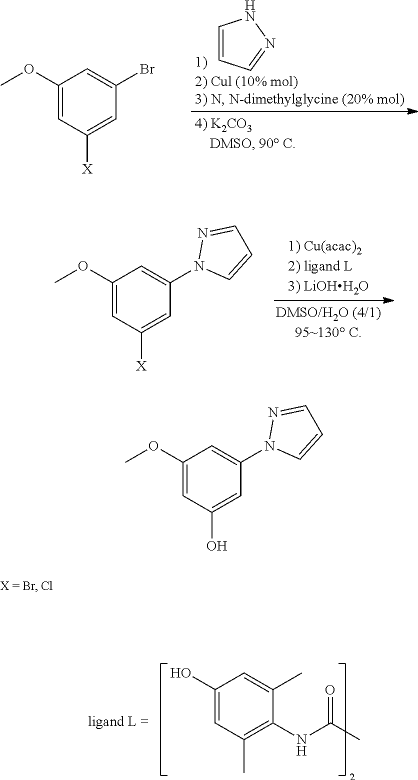

Step 1: A resealable tube was charged with CuI (95 mg, 0.5 mmol, 10% mol), N,N-dimethylglycine (103 mg, 1 mmol, 20% mol), K₂CO₃, aryl halide (5 mmol, 1 eq) and 1H-pyrazole (5 mmol, 1 eq), evacuated and backfilled with nitrogen. To this mixture was added DMSO (10 ml) by syringe at room temperature under nitrogen. The mixture was heated at 90° C. for 24 h. Then it was partitioned between water and ethyl acetate. The organic layer was separated and the aqueous layer was extracted with ethyl acetate. The combined organic layer was washed with brine, dried over Na₂SO₄, and concentrated in vacuo. The residue was loaded on a silica gel column and eluted with 1/10 to 1/5 ethyl acetate/petroleum ether to afford the corresponding coupling product (yield=40%˜70%, purity>90%).

1-(3-bromo-5-methoxyphenyl)-1H-pyrazole:

TLC R_{f}=0.45 (PE/EA=10/1)

MS (ESI⁺): m/z=253.30 (M+1)

1-(3-chloro-5-methoxyphenyl)-1H-pyrazole:

TLC R_{f}=0.48 (PE/EA=10/1)

MS (ESI⁺): m/z=209.40 (M+1)

Step 2: The aryl halide (5 mmol, 1 eq), Cu(acac)₂ (13 mg, 0.05 mmol, 1% mol), LiOH·H₂O (441 mg, 10.5 mmol, 2.1 eq) and ligand L (32.8 mg, 0.1 mmol, 2% mol) were placed into a tube with a magnetic stir bar. The reaction vessel was evacuated and backfilled with nitrogen three times, then DMSO (4 ml) and degassed water (1 ml) were added under a positive nitrogen pressure. The reaction mixture was heated at 90° C. (X═Br)/130° C. (X═Cl) for 24 h under vigorous stirring. The cooled solution was acidified with 2N HCl, then diluted with ethyl acetate (50 ml) and washed with brine (30 ml, 3 times). The organic phase was dried over Na₂SO₄ and concentrated in vacuo. The residue was purified by silica gel flash chromatography to afford the corresponding product (yield=40%˜50%, purity>90%). 3-methoxy-5-(1H-pyrazol-1-yl)phenol:

TLC R_{f}=0.2 (PE/EA=4/1)

MS (ESI⁺): m/z=191.03 (M+1)

### Summary of Intermediates Synthesized

| | | Synthetic |
|---|---|---|
| Intermediates | Structure | method |
| 2-fluoro-3, 4′-bipyridine | 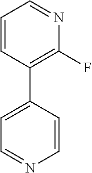 [SMILES: Fc1ncccc1-c1ccncc1] | A |
| 4-(2- fluoropyridin- 3-yl)pyrimidin- 2-amine | 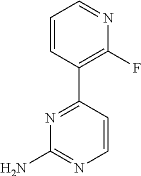 [SMILES: Nc1nccc(-c2cccnc2F)n1] | A |
| 2-fluoro-2′- methoxy-3,4′- bipyridine | 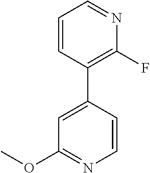 | A |
| 2-fluoro-2′- methyl-3,4′- bipyridine | 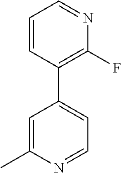 | A |
| 2′-chloro-2- fluoro-3,4′- bipyridine | 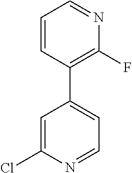 [SMILES: Fc1ncccc1-c1ccnc(Cl)c1] | A |
| 2-fluoro-3,3′- bipyridine | 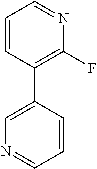 [SMILES: Fc1ncccc1-c1cccnc1] | A |
| 2-fluoro-3- phenylpyridine | 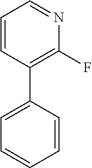 [SMILES: Fc1ncccc1-c1ccccc1] | A |
| 4-(2- fluoropyridin-3- yl)-6- methoxypyrimidine | 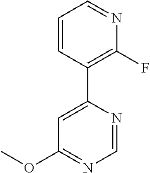 | A |
| 2-fluoro-[3,4′- bipyridin]-2′- amine | 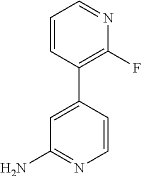 [SMILES: Nc1cc(-c2cccnc2F)ccn1] | A |
| 4-(2- fluoropyridin- 3-yl)-2- (methylthio) pyrimidine | 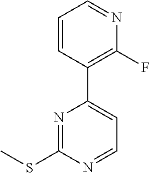 | A |
| 4-(2- fluoropyridin- 3-yl)-6- methylpyrimidine | 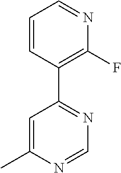 | A |
| 6-(2- fluoropyridin- 3-yl)-N- methylpyrimidin- 4-amine | 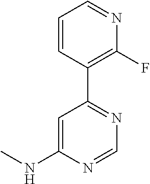 | A |
| 2-fluoro-[3,4′- bipyridin]-3′-amine | 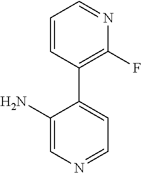 [SMILES: Nc1cnccc1-c1cccnc1F] | A |
| 2-fluoro-2′- (trifluoromethyl)- 3,4′- bipyridine | 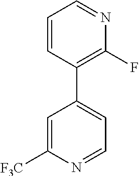 [SMILES: Fc1ncccc1-c1ccnc(C(F)(F)F)c1] | A |
| 2-chloro-4-(2- fluoropyridin-3- yl)pyrimidine | 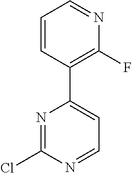 [SMILES: Fc1ncccc1-c1ccnc(Cl)n1] | A |
| 2′-(2- ethoxyethoxy)- 2-fluoro-3,4′- bipyridine | 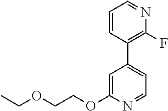 [SMILES: CCOCCOc1cc(-c2cccnc2F)ccn1] | A |
| 2-chloro-3- (pyridin-4-yl) pyrazine | 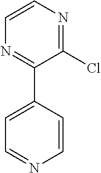 [SMILES: Clc1nccnc1-c1ccncc1] | B |
| 2-chloro-3-(2- methoxypyridin- 4- yl)pyrazine | 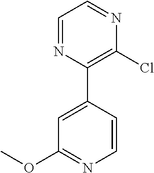 | B |
| 2-chloro-3- (pyridin-3-yl) pyrazine | 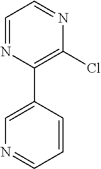 [SMILES: Clc1nccnc1-c1cccnc1] | B |
| 2-chloro-3-(3,5- dimethoxyphenoxy) pyrazine | 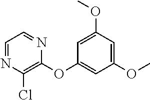 [SMILES: COc1cc(OC)cc(Oc2nccnc2Cl)c1] | C |
| 2-(3,5- dimethoxyphenoxy)- 3- iodopyridine | 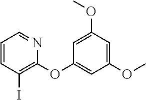 [SMILES: COc1cc(OC)cc(Oc2ncccc2I)c1] | C |
| 2-chloro-3- (1H-imidazol-1- yl)pyrazine | 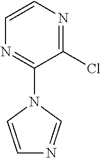 [SMILES: Clc1nccnc1-n1ccnc1] | C |
| 1-(3-chloro-5- methoxyphenyl)- 1H-pyrazole | 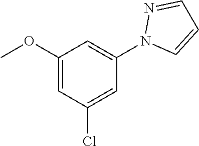 | D |
| 1-(3-bromo-5- methoxyphenyl)- 1H-pyrazole | 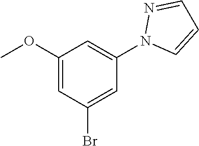 | D |
| 3-methoxy-5- (1H-pyrazol-1- yl)phenol | 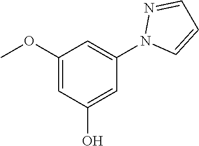 | D |

### EXAMPLES

### Example 1

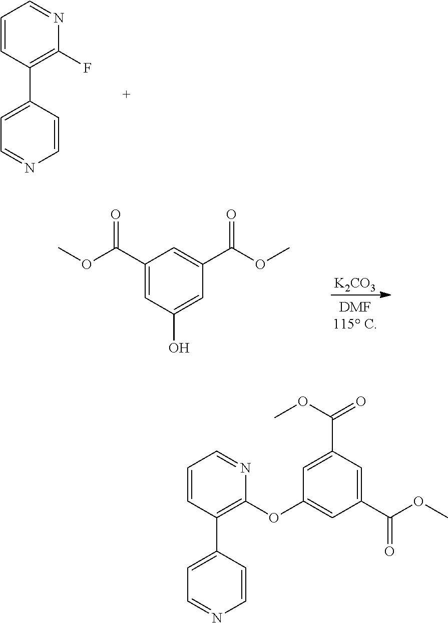

Dimethyl 5-([3,4′-bipyridin]-2-yloxy)isophthalate (Compound 1): 2-fluoro-3,4′-bipyridine (87 mg, 0.5 mmol, 1.0 eq), dimethyl 5-hydroxyisophthalate (105 mg, 0.5 mmol, 1.0 eq) and K₂CO₃ (138 mg, 1.0 mmol, 2 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 8 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product as a white solid. (110 mg, yield=60.4%, purity=98%)

TLC R_{f}=0.25 (PE/EA=1/1)

MS (ESI⁺): m/z=365.40 (M+1)

¹H NMR: (400 MHz, DMSO-d6) δ 8.67 (dt, J=4.5, 1.4 Hz, 2H), 8.31 (dt, J=3.2, 1.5 Hz, 1H), 8.19 (dt, J=4.9, 1.9 Hz, 1H), 8.08 (dt, J=7.6, 2.3 Hz, 1H), 7.98 (dd, J=2.7, 1.5 Hz, 2H), 7.83-7.72 (m, 2H), 7.34 (ddd, J=7.4, 4.9, 2.4 Hz, 1H), 3.88 (d, J=1.7 Hz, 6H). ¹³C NMR: NMR (101 MHz, DMSO) δ 164.74, 158.97, 153.78, 149.71, 147.33, 142.91, 140.46, 131.64, 126.79, 125.73, 123.81, 122.18, 120.22, 52.60, 39.47.

### Example 2

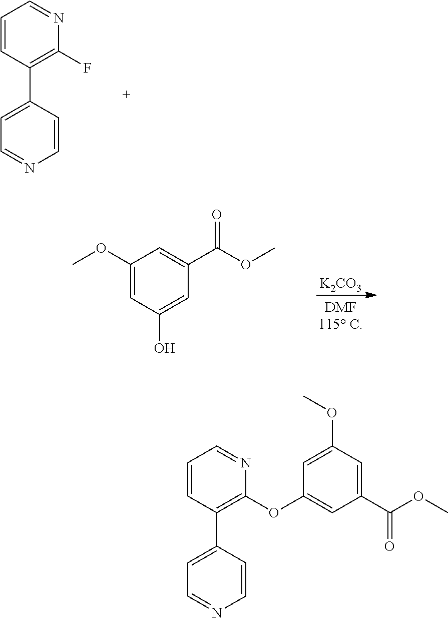

Methyl 3-([3,4′-bipyridin]-2-yloxy)-5-methoxybenzoate (Compound 2): 2-fluoro-3,4′-bipyridine (104 mg, 0.6 mmol, 1.0 eq), methyl 3-hydroxy-5-methoxybenzoate (109 g, 0.6 mmol, 1.0 eq) and K₂CO₃ (165 mg, 1.2 mmol, 2 eq) were added to a round-bottom flamsk with a magnetic bar, then 3 ml DMF was added as solvent. The reaction vessel was evacuated and backfilled with N₂ three times and protected with a balloon of N₂. The reaction mixture was heated at 115° C. for at least 8 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product as a white solid. (183 mg, yield=90.8%, purity=99.6%)

TLC R_{f}=0.33 (PE/EA=1/1)

MS (ESI⁺): m/z=337.40 (M+1)

¹H NMR: (400 MHz, DMSO-d6) δ 8.67 (d, J=5.1 Hz, 2H), 8.21 (dd, J=4.9, 1.9 Hz, 1H), 8.06 (ddq, J=8.4, 4.4, 2.0 Hz, 1H), 7.78-7.65 (m, 2H), 7.31 (tdd, J=7.4, 3.6, 1.9 Hz, 3H), 7.10 (t, J=2.3 Hz, 1H), 3.82 (dd, J=9.8, 1.7 Hz, 6H). ¹³C NMR: (101 MHz, DMSO-d6) δ 165.44, 160.39, 149.73, 147.55, 143.08, 140.29, 131.62, 123.75, 120.00, 114.48, 112.66, 110.61, 55.73, 52.35.

### Example 3

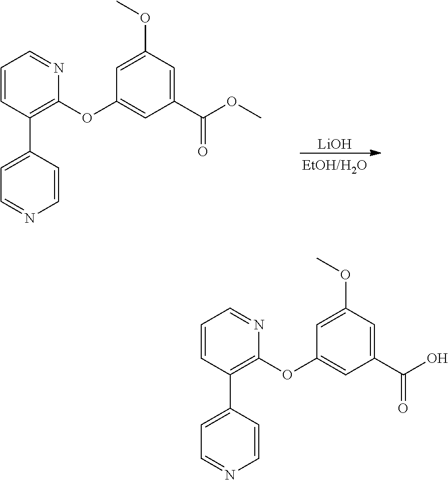 [SMILES: COC(=O)c1cc(OC)cc(Oc2ncccc2-c2ccncc2)c1 ; "LiOH" ; COc1cc(Oc2ncccc2-c2ccncc2)cc(C(=O)O)c1]

3-([3,4′-bipyridin]-2-yloxy)-5-methoxybenzoic acid (Compound 3): methyl 3-([3,4′-bipyridin]-2-yloxy)-5-methoxybenzoate (50 mg, 0.149 mmol, 1.0 eq) and LiOH (18.7 mg, 0.446 mmol, 3.0 eq) were added to a round-bottom flask with a magnetic bar. Then 0.6 ml EtOH and 0.3 ml H₂O were added as solvent. The reaction mixture was stirred overnight. When methyl 3-([3,4′-bipyridin]-2-yloxy)-5-methoxybenzoate was consumed, the pH of reaction mixture was adjusted to 7 and some white solid formed, which was filtered and dried to give the product without further purification. (25 mg, yield=52.1%, purity=96.57%)

MS (ESI⁺): m/z=323.6 (M+1)

### Example 4

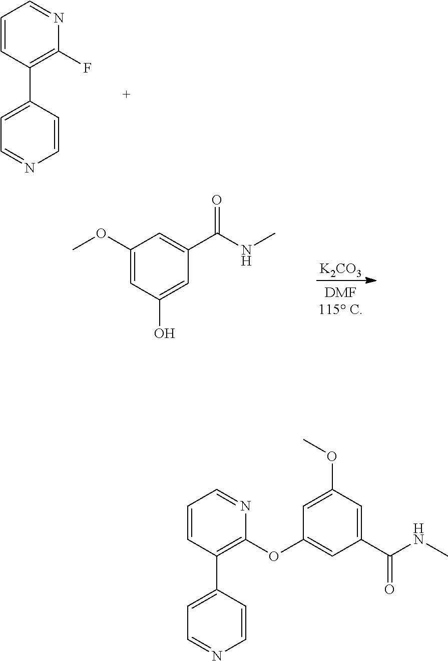

3-([3,4′-bipyridin]-2-yloxy)-5-methoxy-N-methylbenzamide (Compound 4): 2-fluoro-3,4′-bipyridine (87 mg, 0.5 mmol, 1.0 eq), 3-hydroxy-5-methoxy-N-methylbenzamide (91 mg, 0.5 mmol, 1.0 eq) and K₂CO₃ (166 mg, 1.0 mmol, 2 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 8 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product as a white solid. (113 mg, yield=67.5%, purity=99.6%)

TLC R_{f}=0.33 (PE/EA=1/4)

MS (ESI⁺): m/z=336.60 (M+1)

¹H NMR: (400 MHz, DMSO-d6) δ 8.74-8.64 (m, 2H), 8.44 (q, J=4.4 Hz, 1H), 8.21 (dd, J=4.8, 1.9 Hz, 1H), 8.06 (dd, J=7.5, 1.9 Hz, 1H), 7.78-7.68 (m, 2H), 7.31 (dd, J=7.5, 4.9 Hz, 1H), 7.27 (dd, J=2.4, 1.4 Hz, 1H), 7.19 (dd, J=2.1, 1.4 Hz, 1H), 6.96 (t, J=2.2 Hz, 1H), 3.80 (s, 3H), 2.76 (d, J=4.5 Hz, 3H). ¹³C NMR: (101 MHz, DMSO) δ 165.48, 160.20, 159.34, 154.61, 149.75, 147.65, 143.17, 140.22, 136.57, 123.70, 122.11, 119.89, 112.40, 110.30, 108.98, 55.61, 26.22.

### Example 5

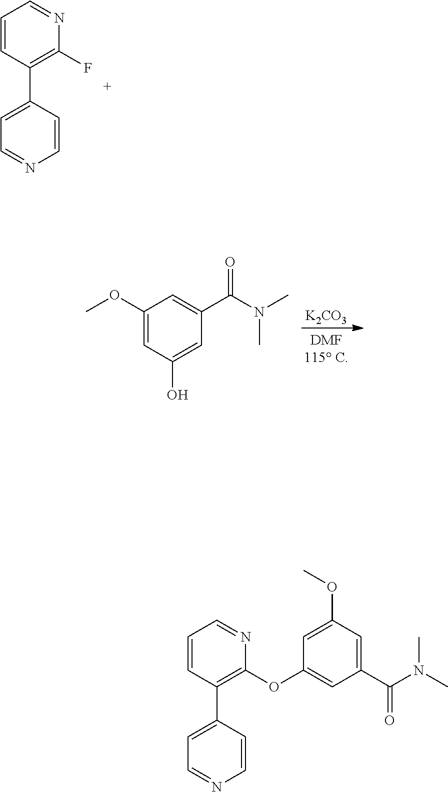

3-([3,4′-bipyridin]-2-yloxy)-5-methoxy-N,N-dimethylbenzamide (Compound 5): 2-fluoro-3,4′-bipyridine (104.4 mg, 0.6 mmol, 1.0 eq), 3-hydroxy-5-methoxy-N,N-dimethylbenzamide (117.1 mg, 0.6 mmol, 1.0 eq) and K₂CO₃ (166 mg, 1.0 mmol, 2 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 8 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product as a white solid. (191 mg, yield=91.2%, purity=99.7%)

TLC R_{f}=0.3 (PE/EA=1/4)

MS (ESI⁺): m/z=350.60 (M+1).

¹H NMR: (400 MHz, DMSO-d6) δ 8.79-8.58 (m, 2H), 8.21 (dd, J=4.8, 1.9 Hz, 1H), 8.05 (dd, J=7.4, 1.9 Hz, 1H), 7.82-7.64 (m, 2H), 7.31 (dd, J=7.5, 4.9 Hz, 1H), 6.85 (t, J=2.2 Hz, 1H), 6.79 (dd, J=2.4, 1.3 Hz, 1H), 6.75 (dd, J=2.1, 1.3 Hz, 1H), 3.77 (s, 3H), 2.92 (d, J=17.8 Hz, 6H). ¹³C NMR: (101 MHz, DMSO) b 168.99, 160.15, 159.29, 154.48, 149.71, 147.61, 143.18, 140.22, 138.42, 123.73, 122.17, 119.90, 112.19, 108.83, 108.34, 55.58, 54.87, 34.62.

### Example 6

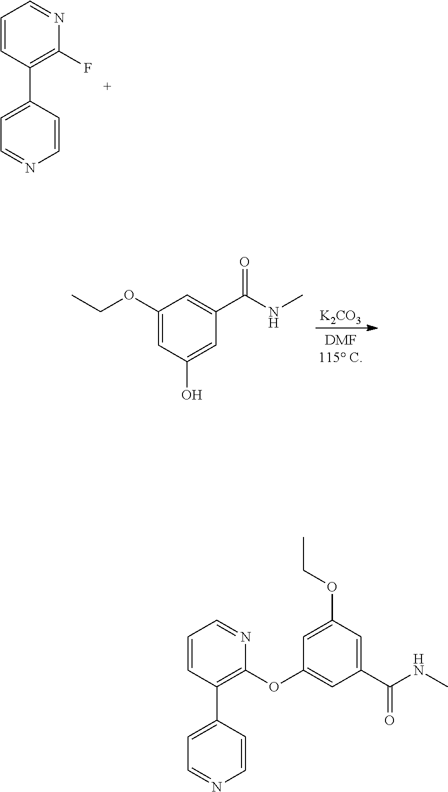

3-([3,4′-bipyridin]-2-yloxy)-5-ethoxy-N-methylbenzamide (Compound 6): 2-fluoro-3,4′-bipyridine (104.4 mg, 0.6 mmol, 1.0 eq), 3-ethoxy-5-hydroxy-N-methylbenzamide (117.1 mg, 0.6 mmol, 1.0 eq) and K₂CO₃ (166 mg, 1.0 mmol, 2 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 8 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product as a white solid. (168 mg, yield=80.1%, purity=83%)

TLC R_{f}=0.3 (PE/EA=1/4)

MS (ESI⁺): m/z=350.40 (M+1).

### Example 7

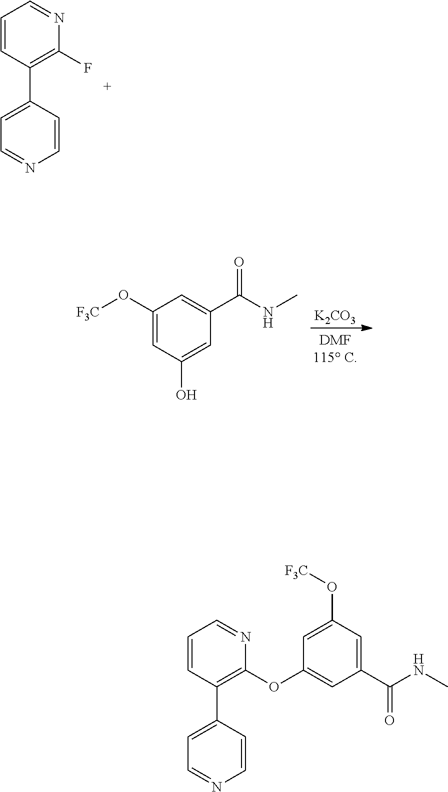

3-([3,4′-bipyridin]-2-yloxy)-N-methyl-5-(trifluoromethoxy)benzamide (Compound 7): 2-fluoro-3,4′-bipyridine (104.4 mg, 0.6 mmol, 1.0 eq), 3-hydroxy-N-methyl-5-(trifluoromethoxy)benzamide (141 mg, 0.6 mmol, 1.0 eq) and K₂CO₃ (166 mg, 1.0 mmol, 2 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 8 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product as a white solid. (193 mg, yield=82.6%, purity=99.6%)

TLC R_{f}=0.3 (PE/EA=1/4)

MS (ESI⁺): m/z=390.30 (M+1).

¹H NMR: (400 MHz, DMSO-d6) δ 8.74-8.67 (m, 2H), 8.64 (q, J=4.5 Hz, 1H), 8.24 (dd, J=4.9, 1.9 Hz, 1H), 8.10 (dd, J=7.5, 1.9 Hz, 1H), 7.80-7.74 (m, 2H), 7.71 (t, J=1.8 Hz, 1H), 7.68 (dt, J=2.4, 1.2 Hz, 1H), 7.54 (t, J=2.1 Hz, 1H), 7.36 (dd, J=7.5, 4.8 Hz, 1H), 2.79 (d, J=4.5 Hz, 3H). ¹³C NMR: (101 MHz, DMSO) b 164.64, 159.47, 154.98, 150.27, 149.22, 148.06, 143.43, 140.97, 137.72, 124.23, 122.68, 120.80, 119.98, 118.13, 116.27, 26.78.

### Example 8

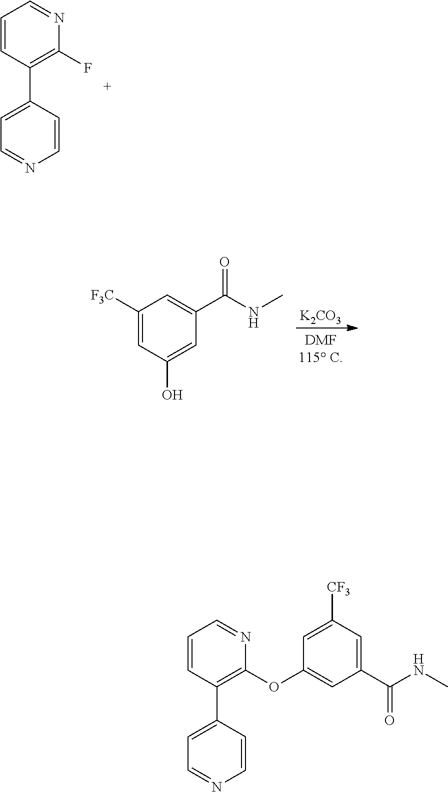

3-([3,4′-bipyridin]-2-yloxy)-N-methyl-5-(trifluoromethyl)benzamide (Compound 8): 2-fluoro-3,4′-bipyridine (104.4 mg, 0.6 mmol, 1.0 eq), 3-hydroxy-N-methyl-5-(trifluoromethyl)benzamide (131.4 mg, 0.6 mmol, 1.0 eq) and K₂CO₃ (166 mg, 1.0 mmol, 2 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 8 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product as a white solid. (130 mg, yield=58%, purity=98%)

TLC R_{f}=0.3 (PE/EA=1/1)

MS (ESI⁺): m/z=374.40 (M+1).

### Example 9

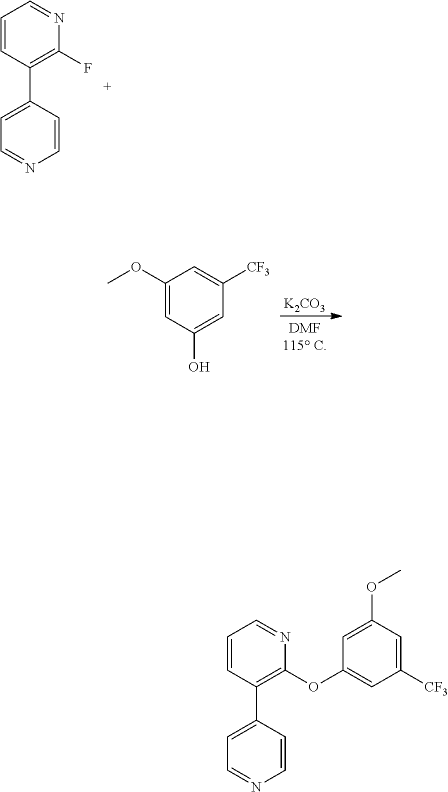

2-(3-methoxy-5-(trifluoromethyl)phenoxy)-3,4′-bipyridine (Compound 9): 2-fluoro-3,4′-bipyridine (87 mg, 0.5 mmol, 1.0 eq), 3-methoxy-5-(trifluoromethyl)phenol (96 mg, 0.5 mmol, 1.0 eq) and K₂CO₃ (166 mg, 1.0 mmol, 2 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 8 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product as a white solid. (108 mg, yield=62.4%, purity=91.6%) TLC R_{f}=0.3 (PE/EA=1/1)

MS (ESI⁺): m/z=347.40 (M+1).

¹H NMR: (400 MHz, DMSO-d6) δ 8.69 (d, J=5.2 Hz, 2H), 8.22 (dd, J=4.9, 1.8 Hz, 1H), 8.06 (dd, J=7.5, 1.9 Hz, 1H), 7.82-7.71 (m, 2H), 7.32 (dd, J=7.5, 4.8 Hz, 1H), 7.19 (t, J=1.7 Hz, 1H), 7.13 (dt, J=9.1, 2.1 Hz, 2H), 3.83 (s, 3H). ¹³C NMR: (101 MHz, DMSO) δ 161.28, 159.66, 155.56, 150.20, 147.98, 143.55, 140.78, 131.57, 125.46, 124.26, 122.60, 120.52, 112.32, 111.34, 107.79, 56.41.

### Example 10

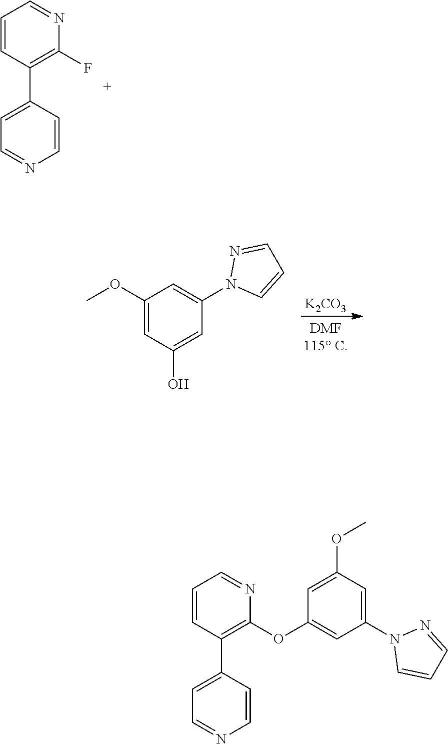

2-(3-methoxy-5-(1H-pyrazol-1-yl)phenoxy)-3,4′-bipyridine (Compound 10): 2-fluoro-3,4′-bipyridine (87 mg, 0.5 mmol, 1.0 eq), 3-methoxy-5-(1H-pyrazol-1-yl)phenol (95.1 mg, 0.5 mmol, 1.0 eq) and K₂CO₃ (166 mg, 1.0 mmol, 2 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 8 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product as a white solid. (164 mg, yield=95.2%, purity=99%)

TLC R_{f}=0.3 (PE/EA=1/2)

MS (ESI⁺): m/z=345.50 (M+1).

### Example 11

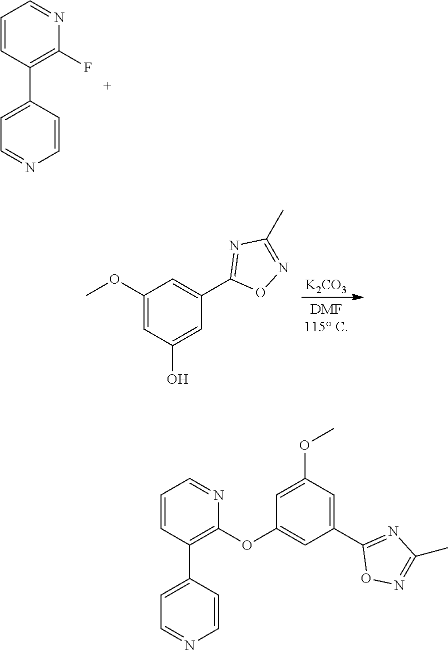

5-(3-([3,4′-bipyridin]-2-yloxy)-5-methoxyphenyl)-3-methyl-1,2,4-oxadiazole (Compound 11): 2-fluoro-3,4′-bipyridine (87 mg, 0.5 mmol, 1.0 eq), 3-methoxy-5-(3-methyl-1,2,4-oxadiazol-5-yl)phenol (103 mg, 0.5 mmol, 1.0 eq) and K₂CO₃ (166 mg, 1.0 mmol, 2 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 8 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product as a white solid. (152 mg, yield=84.4%, purity=95%)

TLC R_{f}=0.45 (PE/EA=1/2)

MS (ESI⁺): m/z=361.40 (M+1).

¹H NMR: (400 MHz, DMSO-d6) δ 8.74-8.63 (m, 2H), 8.22 (dd, J=4.8, 1.9 Hz, 1H), 8.08 (dd, J=7.5, 1.9 Hz, 1H), 7.82-7.71 (m, 2H), 7.46 (t, J=1.8 Hz, 1H), 7.42 (dd, J=2.4, 1.4 Hz, 1H), 7.34 (dd, J=7.5, 4.9 Hz, 1H), 7.14 (t, J=2.3 Hz, 1H), 3.85 (s, 3H), 2.40 (s, 3H). ¹³C NMR: (101 MHz, DMSO) δ 174.53, 168.19, 161.38, 159.58, 155.79, 150.20, 148.05, 143.57, 140.85, 125.54, 124.27, 122.73, 120.63, 113.61, 112.97, 109.59, 56.37, 11.68.

### Example 12

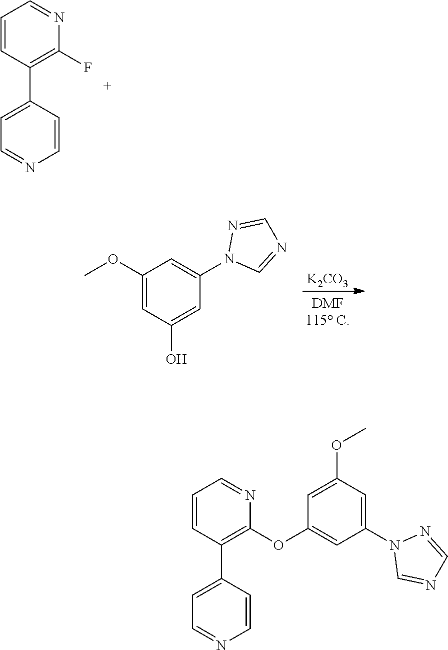

2-(3-methoxy-5-(1H-1,2,4-triazol-1-yl)phenoxy)-3,4′-bipyridine (Compound 12): 2-fluoro-3,4′-bipyridine (87 mg, 0.5 mmol, 1.0 eq), 3-methoxy-5-(1H-1,2,4-triazol-1-yl)phenol (95.5 mg, 0.5 mmol, 1.0 eq) and K₂CO₃ (166 mg, 1.0 mmol, 2 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 8 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product as a white solid. (12 mg, yield=6.9%, purity=83%)

TLC R_{f}=0.33 (PE/EA=1/2)

MS (ESI⁺): m/z=346.4 (M+1).

### Example 13

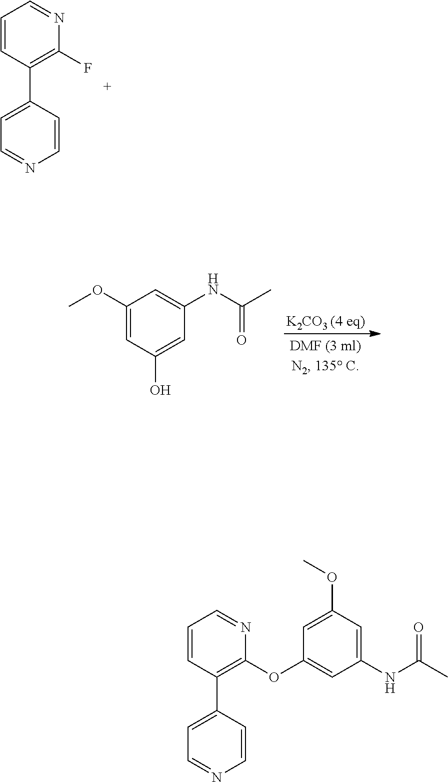 [SMILES: Fc1ncccc1-c1ccncc1 ; COc1cc(O)cc(NC(C)=O)c1 ; "K2CO3 (4 eq)" ; COc1cc(NC(C)=O)cc(Oc2ncccc2-c2ccncc2)c1]

N-(3-([3,4′-bipyridin]-2-yloxy)-5-methoxyphenyl)acetamide (Compound 13): 2-fluoro-3,4′-bipyridine (87 mg, 0.5 mmol, 1.0 eq), N-(3-hydroxy-5-methoxyphenyl)acetamide (90.5 mg, 0.5 mmol, 1.0 eq) and K₂CO₃ (276 mg, 2 mmol, 4.0 eq) were added to a round-bottom flask with a magnetic bar, then 3 ml DMF was added as solvent. The reaction vessel was evacuated and backfilled with N₂ three times and protected with a balloon of N₂. The reaction mixture was heated at 135° C. for at least 8 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product as a white solid. (86.6 mg, yield=49.3%, purity=99%).

TLC R_{f}=0.30 (PE/EA=1/2)

MS (ESI⁺): m/z=336.40 (M+1)

¹H NMR: (400 MHz, DMSO-d6) δ 10.00 (s, 1H), 9.04-8.53 (m, 2H), 8.23 (d, J=4.7 Hz, 1H), 8.05 (d, J=7.5 Hz, 1H), 7.70 (d, J=5.1 Hz, 2H), 7.31 (t, J=6.0 Hz, 1H), 7.03 (d, J=16.7 Hz, 2H), 6.48 (s, 1H), 3.72 (s, 3H), 2.02 (s, 3H). ¹³C NMR: (101 MHz, DMSO) δ 168.95, 160.81, 159.82, 155.51, 150.24, 148.27, 143.70, 141.38, 140.67, 124.15, 122.77, 120.38, 104.68, 102.32, 101.46, 55.75, 24.57.

### Example 14

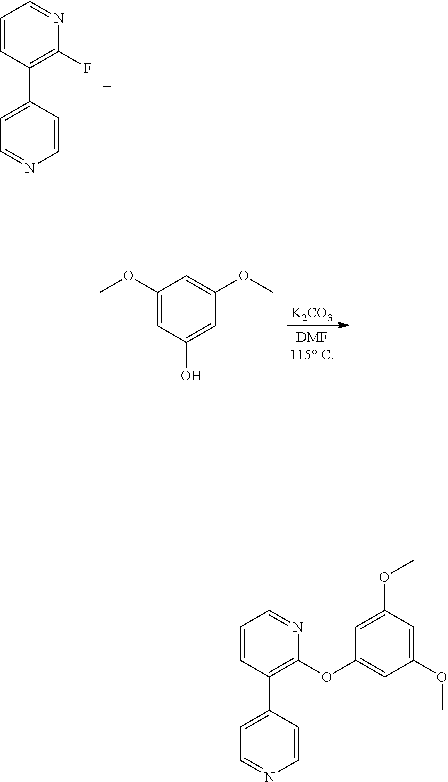

2-(3,5-dimethoxyphenoxy)-3,4′-bipyridine (Compound 14): 2-fluoro-3,4′-bipyridine (104 mg, 0.6 mmol, 1.0 eq), 3,5-dimethoxyphenol (92 mg, 0.6 mmol, 1.0 eq) and K₂CO₃ (166 mg, 1.2 mmol, 2 eq) were added to a round-bottom flask with a magnetic bar, then 6 ml DMF was added as solvent. The reaction vessel was evacuated and backfilled with N₂ three times and protected with a balloon of N₂. The reaction mixture was heated at 115° C. for at least 8 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product as a colorless oil. (162 mg, yield=88%, purity=95.9%)

TLC R_{f}=0.25 (PE/EA=1/1)

MS (ESI⁺): m/z=309.40 (M+1)

¹H NMR: (400 MHz, DMSO-d6) δ 8.73-8.63 (m, 2H), 8.21 (dd, J=4.8, 1.9 Hz, 1H), 8.08-7.98 (m, 1H), 7.75-7.68 (m, 2H), 7.37-7.22 (m, 1H), 6.35 (tt, J=3.4, 1.3 Hz, 3H), 3.71 (s, 6H). ¹³C NMR: (101 MHz, DMSO) δ 160.98, 159.38, 155.49, 150.07, 149.71, 147.68, 143.25, 140.08, 123.70, 122.14, 119.73, 99.96, 96.77, 55.35.

### Example 15

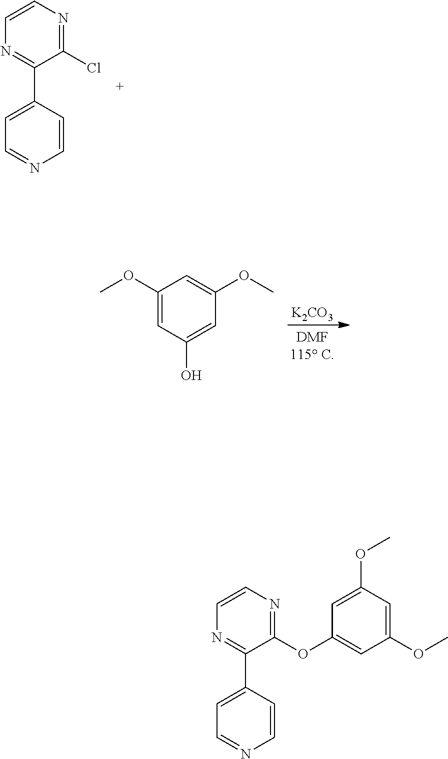

2-(3,5-dimethoxyphenoxy)-3-(pyridin-4-yl)pyrazine (Compound 15): 2-chloro-3-(pyridin-4-yl)pyrazine (130 mg, 0.68 mmol, 1.0 eq), 3,5-dimethoxyphenol (154 mg, 0.68 mmol, 1.0 eq) and K₂CO₃ (188 mg, 1.36 mmol, 2.0 eq) were added to a round-bottom flask with a magnetic bar, then 6 ml DMF was added as solvent. The reaction vessel was evacuated and backfilled with N₂ three times and protected with a balloon of N₂. The reaction mixture was heated at 115° C. for at least 8 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product as a white solid. (95 mg, yield=45.3%, purity=97.9%)

TLC R_{f}=0.25 (PE/EA=2/1)

MS (ESI⁺): m/z=310.50 (M+1)

¹H NMR: (400 MHz, DMSO-d6) δ 8.75 (d, J=5.7 Hz, 2H), 8.54 (t, J=2.0 Hz, 1H), 8.29 (d, J=2.5 Hz, 1H), 8.12-7.94 (m, 2H), 6.48 (d, J=2.3 Hz, 2H), 6.41 (t, J=2.2 Hz, 1H), 3.73 (s, 6H). ¹³C NMR: (101 MHz, DMSO) δ 161.09, 157.40, 154.44, 149.90, 142.32, 141.74, 139.86, 139.03, 123.01, 100.22, 97.49, 55.44.

### Example 17

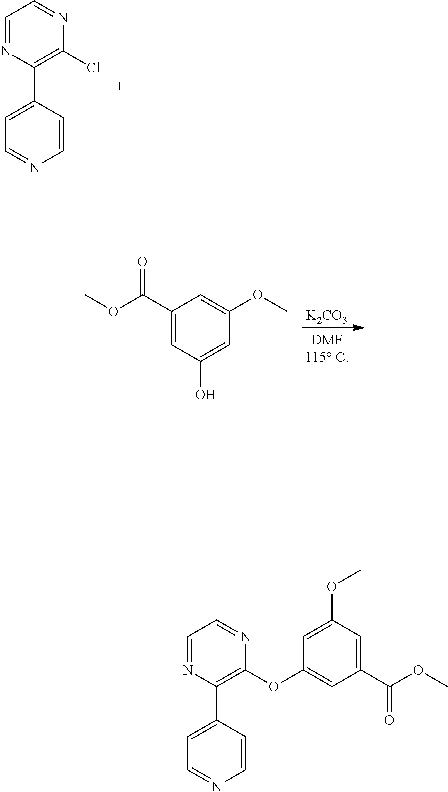

Methyl 3-([3,4′-bipyridin]-2-yloxy)-5-methoxybenzoate (Compound 17): 2-chloro-3-(pyridin-4-yl)pyrazine (115 mg, 0.6 mmol, 1.0 eq), methyl 3-hydroxy-5-methoxybenzoate (110 mg, 0.6 mmol, 1.0 eq) and K₂CO₃ (138 mg, 1.0 mmol, 2 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 8 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product as a white solid. (166.8 mg, yield=82.7%, purity=99.5%).

TLC R_{f}=0.2 (PE/EA=2/1)

MS (ESI⁺): m/z=338.50 (M+1)

¹H NMR: (400 MHz, DMSO-d6) δ 8.81-8.67 (m, 2H), 8.56 (d, J=2.5 Hz, 1H), 8.27 (d, J=2.5 Hz, 1H), 8.14-8.01 (m, 2H), 7.43 (dd, J=2.1, 1.4 Hz, 1H), 7.36 (dd, J=2.4, 1.4 Hz, 1H), 7.24 (t, J=2.3 Hz, 1H), 3.83 (d, J=9.8 Hz, 6H). ¹³C NMR: (101 MHz, DMSO) δ 165.34, 160.45, 157.24, 153.70, 149.90, 142.23, 141.50, 140.00, 139.22, 131.80, 123.08, 114.81, 112.98, 111.39, 55.81, 52.39.

### Example 18

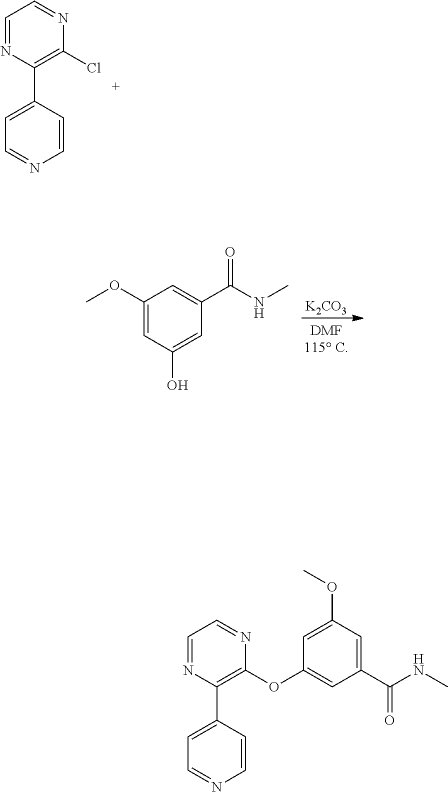

3-methoxy-N-methyl-5-((3-(pyridin-4-yl)pyrazin-2-yl)oxy)benzamide (Compound 18): 2-chloro-3-(pyridin-4-yl)pyrazine (95.5 mg, 0.5 mmol, 1.0 eq), 3-hydroxy-5-methoxy-N-methylbenzamide (90.5 mg, 0.5 mmol, 1.0 eq) and K₂CO₃ (276 mg, 2 mmol, 4.0 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 8 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product. (166.6 mg, yield=99.2%, purity=99.4%).

TLC R_{f}=0.20 (PE/EA=1/2)

MS (ESI⁺): m/z=337.40 (M+1)

¹H NMR: ¹H NMR (400 MHz, DMSO-d6) δ 8.87-8.69 (m, 2H), 8.63-8.52 (m, 1H), 8.45 (d, J=5.2 Hz, 1H), 8.36-8.24 (m, 1H), 8.20-7.99 (m, 2H), 7.42-7.25 (m, 2H), 7.10 (t, J=2.2 Hz, 1H), 3.81 (s, 3H), 2.77 (d, J=4.4 Hz, 3H). ¹³C NMR: (101 MHz, DMSO) δ 165.38, 160.30, 157.30, 153.63, 149.94, 142.24, 141.70, 139.93, 139.25, 136.78, 123.00, 112.57, 110.51, 109.72, 55.69, 26.23.

### Example 19

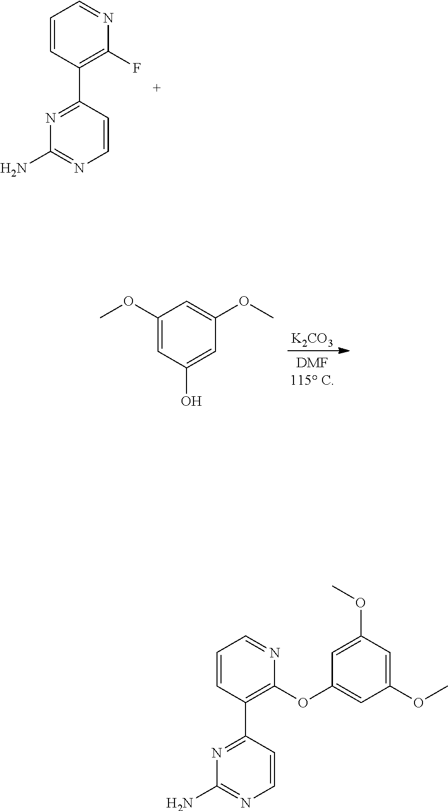

4-(2-(3,5-dimethoxyphenoxy)pyridin-3-yl)pyrimidin-2-amine (Compound 19): 4-(2-fluoropyridin-3-yl)pyrimidin-2-amine (95 mg, 0.5 mmol, 1.0 eq), 3,5-dimethoxyphenol (77 mg, 0.5 mmol, 1.0 eq) and K₂CO₃ (138 mg, 1 mmol, 2 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 8 h with vigorous stirring. The cooled solution was diluted with 20 ml ethyl acetate and washed with brine. The organic phase was dried over anhydrous Na₂SO₄ and concentrated in vacuo. The residue was purified by silica gel flash chromatography to afford the product as a white solid. (112 mg, yield=69.1%, purity=98%).

TLC R_{f}=0.33 (PE/EA=1/1)

MS (ESI⁺): m/z=325.40 (M+1)

¹H NMR: (400 MHz, DMSO-d6) δ 8.37 (dd, J=7.6, 2.0 Hz, 1H), 8.33 (d, J=5.1 Hz, 1H), 8.24 (dd, J=4.8, 2.0 Hz, 1H), 7.29 (dd, J=7.5, 4.8 Hz, 1H), 7.24 (d, J=5.2 Hz, 1H), 6.77 (s, 2H), 6.38 (t, J=2.2 Hz, 1H), 6.34 (d, J=2.2 Hz, 2H), 3.73 (s, 6H). ¹³C NMR: (101 MHz, DMSO) δ 164.27, 161.49, 161.04, 160.59, 159.12, 155.93, 148.97, 140.40, 122.39, 119.96, 110.39, 100.41, 97.27, 55.86.

### Example 21

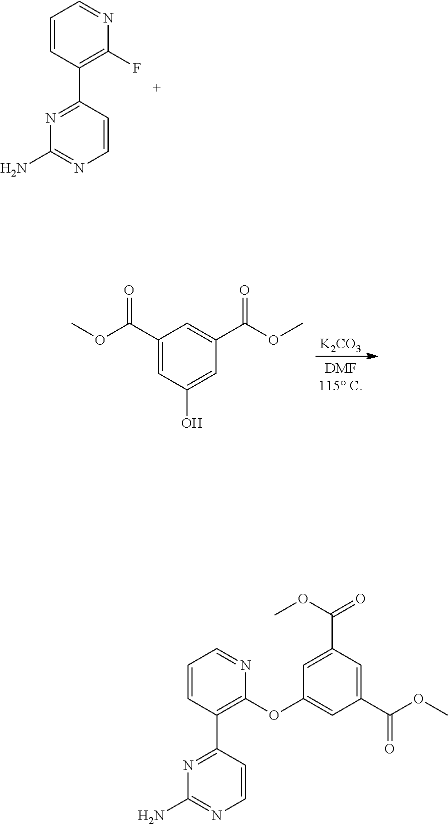

dimethyl 5-((3-(2-aminopyrimidin-4-yl)pyridin-2-yl)oxy)isophthalate (Compound 21): 4-(2-fluoropyridin-3-yl)pyrimidin-2-amine (95 mg, 0.5 mmol, 1.0 eq), dimethyl 5-hydroxyisophthalate (105 mg, 0.5 mmol, 1.0 eq) and K₂CO₃ (138 mg, 1 mmol, 2 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 8 h with vigorous stirring. The cooled solution was diluted with 20 ml ethyl acetate and washed with brine. The organic phase was dried over anhydrous Na₂SO₄ and concentrated in vacuo. The residue was purified by silica gel flash chromatography to afford the product as a white solid. (110 mg, yield=57.9%, purity=93%).

TLC R_{f}=0.33 (PE/EA=1/2)

MS (ESI⁺): m/z=381.40 (M+1)

### Example 22

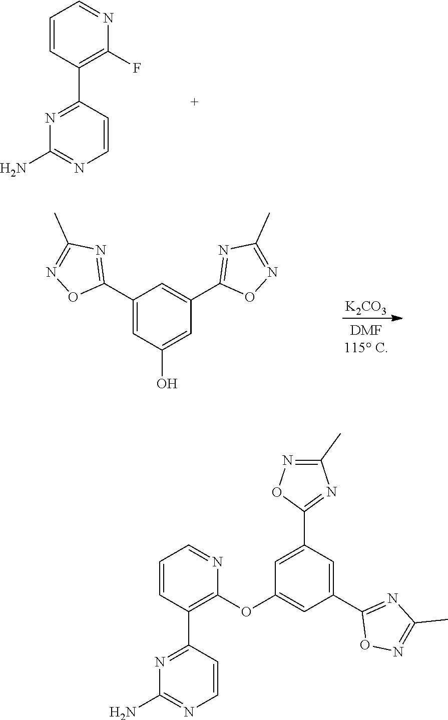

4-(2-(3,5-bis(3-methyl-1,2,4-oxadiazol-5-yl)phenoxy)pyridin-3-yl)pyrimidin-2-amine (Compound 22): 4-(2-fluoropyridin-3-yl)pyrimidin-2-amine (132.6 mg, 0.7 mmol, 1.0 eq), 3,5-bis(3-methyl-1,2,4-oxadiazol-5-yl)phenol (180 mg, 0.7 mmol, 1.0 eq) and K₂CO₃ (138 mg, 1 mmol, 2 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 8 h with vigorous stirring. The cooled solution was diluted with 20 ml ethyl acetate and washed with brine. The organic phase was dried over anhydrous Na₂SO₄ and concentrated in vacuo. The residue was purified by silica gel flash chromatography to afford the product as a white solid. (80 mg, yield=26.7%, purity=92%).

TLC R_{f}=0.25 (PE/EA=2/3)

MS (ESI⁺): m/z=429.50 (M+1)

### Example 23

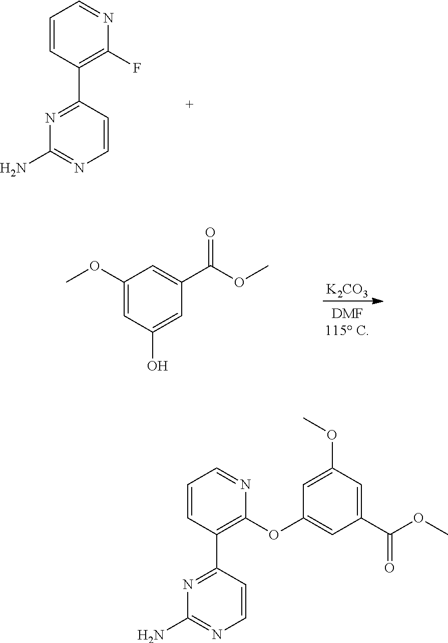

Methyl 3-((3-(2-aminopyrimidin-4-yl)pyridin-2-yl)oxy)-5-methoxybenzoate (Compound 23): 4-(2-fluoropyridin-3-yl)pyrimidin-2-amine (95 mg, 0.5 mmol, 1.0 eq), methyl 3-hydroxy-5-methoxybenzoate (91 mg, 0.5 mmol, 1.0 eq) and K₂CO₃ (138 mg, 1 mmol, 2 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 8 h with vigorous stirring. The cooled solution was diluted with 20 ml ethyl acetate and washed with brine. The organic phase was dried over anhydrous Na₂SO₄ and concentrated in vacuo. The residue was purified by silica gel flash chromatography to afford the product as a white solid. (145 mg, yield=82.4%, purity=99.7%).

TLC R_{f}=0.33 (PE/EA=1/1)

MS (ESI⁺): m/z=353.40 (M+1)

### Example 24

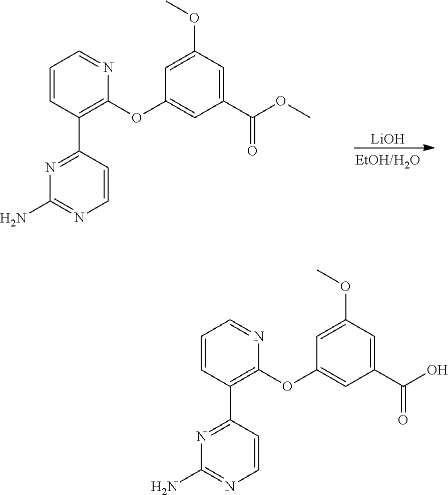 [SMILES: COC(=O)c1cc(OC)cc(Oc2ncccc2-c2ccnc(N)n2)c1 ; "LiOH" ; COc1cc(Oc2ncccc2-c2ccnc(N)n2)cc(C(=O)O)c1]

3-((3-(2-aminopyrimidin-4-yl)pyridin-2-yl)oxy)-5-methoxybenzoic acid (Compound 24): methyl 3-((3-(2-aminopyrimidin-4-yl)pyridin-2-yl)oxy)-5-methoxybenzoate (50 mg, 0.14 mmol, 1.0 eq) and LiOH (10 mg, 0.42 mmol, 3.0 eq) were added to a round-bottom flask with a magnetic bar. Then 0.6 ml EtOH and 0.3 ml H₂O were added as solvent. The reaction mixture was stirred overnight. methyl 3-((3-(2-aminopyrimidin-4-yl)pyridin-2-yl)oxy)-5-methoxybenzoate was consumed, the pH of reaction mixture was adjusted to 7 and some white solid formed, which was filtered and dried to give the product without further purification. (33 mg, yield=69.7%, purity=99%)

MS (ESI⁺): m/z=323.6 (M+1)

### Example 25

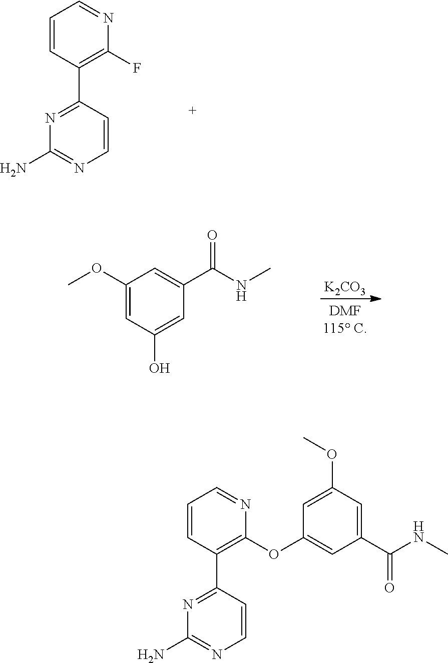

3-((3-(2-aminopyrimidin-4-yl)pyridin-2-yl)oxy)-5-methoxy-N-methylbenzamide (Compound 25): 4-(2-fluoropyridin-3-yl)pyrimidin-2-amine (95 mg, 0.5 mmol, 1.0 eq), 3-hydroxy-5-methoxy-N-methylbenzamide (91 mg, 0.5 mmol, 1.0 eq) and K₂CO₃ (138 mg, 1 mmol, 2 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 8 h with vigorous stirring. The cooled solution was diluted with 20 ml ethyl acetate and washed with brine. The organic phase was dried over anhydrous Na₂SO₄ and concentrated in vacuo. The residue was purified by silica gel flash chromatography to afford the product as a white solid. (118 mg, yield=67.2%, purity=97.5%).

TLC R_{f}=0.3 (PE/EA=1/4)

MS (ESI⁺): m/z=352.50 (M+1)

### Example 26

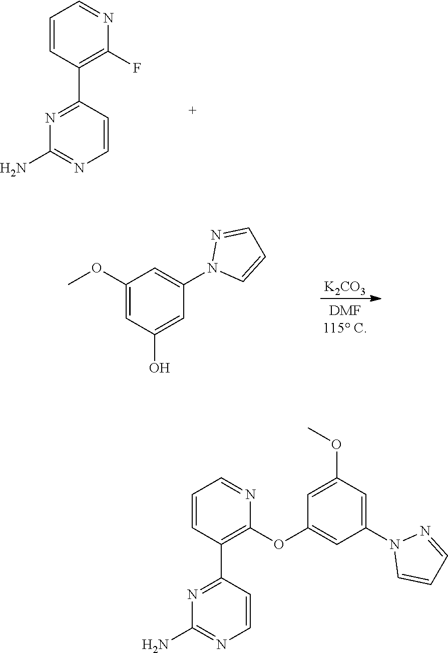

4-(2-(3-methoxy-5-(1H-pyrazol-1-yl)phenoxy)pyridin-3-yl)pyrimidin-2-amine (Compound 26): 4-(2-fluoropyridin-3-yl)pyrimidin-2-amine (95 mg, 0.5 mmol, 1.0 eq), 3-methoxy-5-(1H-pyrazol-1-yl)phenol (91 mg, 0.5 mmol, 1.0 eq) and K₂CO₃ (138 mg, 1 mmol, 2 eq) were added to a round-bottom flask with a magnetic bar, then 3 ml DMF was added as solvent.

The reaction vessel was evacuated and backfilled with N₂ three times and protected with a balloon of N₂. The reaction mixture was heated at 115° C. for at least 8 h with vigorous stirring. The cooled solution was diluted with 20 ml ethyl acetate and washed with brine. The organic phase was dried over anhydrous Na₂SO₄ and concentrated in vacuo. The residue was purified by silica gel flash chromatography to afford the product as a white solid. (130 mg, yield=72.22%, purity=99%).

TLC R_{f}=0.25 (PE/EA=1/1)

MS (ESI⁺): m/z=361.40 (M+1)

¹H NMR: (400 MHz, DMSO-d6) δ 8.56 (d, J=2.6 Hz, 1H), 8.39 (dd, J=7.6, 2.0 Hz, 1H), 8.34 (d, J=5.2 Hz, 1H), 8.25 (dd, J=4.8, 2.0 Hz, 1H), 7.73 (d, J=1.7 Hz, 1H), 7.39-7.31 (m, 2H), 7.30-7.24 (m, 2H), 6.78 (s, 2H), 6.73 (t, J=2.2 Hz, 1H), 6.54 (t, J=2.1 Hz, 1H), 3.83 (s, 3H).

### Example 27

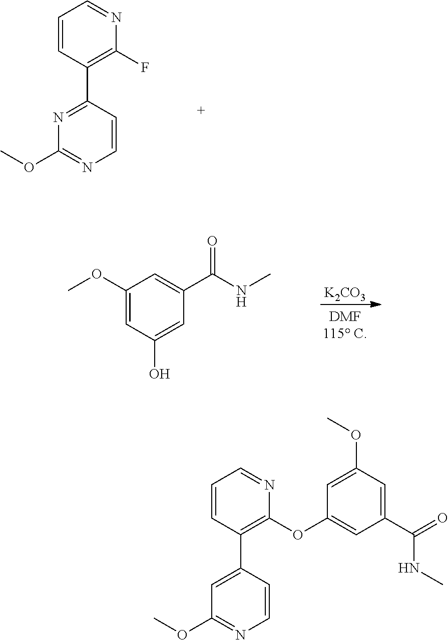

3-methoxy-5-((2′-methoxy-[3,4′-bipyridin]-2-yl)oxy)-N-methylbenzamide (Compound 27): 2-fluoro-2′-methoxy-3,4′-bipyridine (102 mg, 0.5 mmol, 1.0 eq), 3-hydroxy-5-methoxy-N-methylbenzamide (90.5 mg, 0.5 mmol, 1.0 eq) and K₂CO₃ (276 mg, 2 mmol, 4.0 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product as a white solid. (174.2 mg, yield=95.4%, purity=99.1%) TLC R_{f}=0.4 (PE/EA=1/2)

MS (ESI⁺): m/z=366.50 (M+1)

¹H NMR: (400 MHz, DMSO-d6) δ 8.44 (q, J=4.4 Hz, 1H), 8.26 (dd, J=5.4, 0.7 Hz, 1H), 8.20 (dd, J=4.9, 1.9 Hz, 1H), 8.04 (dd, J=7.5, 1.9 Hz, 1H), 7.34-7.23 (m, 3H), 7.18 (dd, J=2.1, 1.4 Hz, 1H), 7.14 (dd, J=1.5, 0.7 Hz, 1H), 6.95 (t, J=2.2 Hz, 1H), 3.89 (s, 3H), 3.80 (s, 3H), 2.76 (d, J=4.5 Hz, 3H). ¹³C NMR: (101 MHz, DMSO) δ 165.47, 163.83, 160.20, 159.30, 154.66, 147.60, 146.88, 146.21, 140.15, 136.57, 122.10, 119.80, 117.36, 112.34, 110.33, 110.26, 108.97, 55.60, 53.19, 26.22.

### Example 28

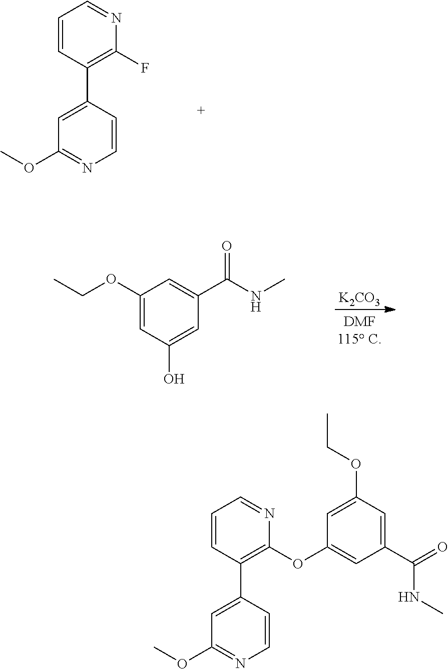

3-ethoxy-5-((2′-methoxy-[3,4′-bipyridin]-2-yl)oxy)-N-methylbenzamide (Compound 28): 2-fluoro-2′-methoxy-3,4′-bipyridine (122.5 mg, 0.6 mmol, 1.0 eq), 3-ethoxy-5-hydroxy-N-methylbenzamide (117 mg, 0.6 mmol, 1.0 eq) and K₂CO₃ (276 mg, 2 mmol, 4.0 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product as a white solid. (192 mg, yield=84.3%, purity=95.77%)

TLC R_{f}=0.25 (PE/EA=1/2)

MS (ESI⁺): m/z=380.50 (M+1)

¹H NMR: (400 MHz, DMSO-d6) δ 8.43 (d, J=4.6 Hz, 1H), 8.26 (d, J=5.4 Hz, 1H), 8.21 (dd, J=4.8, 1.9 Hz, 1H), 8.04 (dd, J=7.5, 1.9 Hz, 1H), 7.36-7.24 (m, 3H), 7.17 (t, J=1.8 Hz, 1H), 7.14 (d, J=1.4 Hz, 1H), 6.93 (t, J=2.2 Hz, 1H), 4.07 (q, J=7.0 Hz, 2H), 3.90 (s, 3H), 2.76 (d, J=4.5 Hz, 3H), 1.33 (t, J=7.0 Hz, 3H). ¹³C NMR: (101 MHz, DMSO) δ 165.98, 164.34, 159.93, 159.80, 155.18, 148.11, 147.38, 146.72, 140.64, 137.03, 122.64, 120.30, 117.86, 112.68, 111.12, 110.83, 109.87, 64.09, 53.69, 26.72, 14.99.

### Example 29

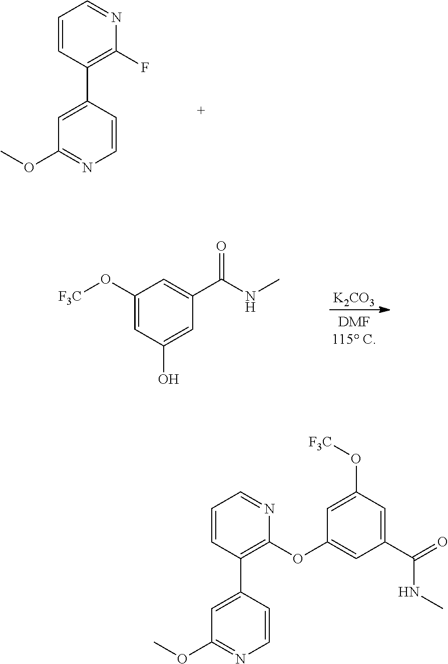

3-((2′-methoxy-[3,4′-bipyridin]-2-yl)oxy)-N-methyl-5-(trifluoromethoxy)benzamide (Compound 29): 2-fluoro-2′-methoxy-3,4′-bipyridine (122.5 mg, 0.6 mmol, 1.0 eq), 3-hydroxy-N-methyl-5-(trifluoromethoxy)benzamide (141 mg, 0.6 mmol, 1.0 eq) and K₂CO₃ (276 mg, 2 mmol, 4.0 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product as a white solid. (181 mg, yield=71.9%, purity=96%)

TLC R_{f}=0.4 (PE/EA=1/1)

MS (ESI⁺): m/z=420.40 (M+1)

¹H NMR: (400 MHz, DMSO-d6) δ 8.64 (q, J=4.5 Hz, 1H), 8.27 (d, J=5.3 Hz, 1H), 8.22 (dd, J=4.9, 1.9 Hz, 1H), 8.08 (dd, J=7.5, 1.9 Hz, 1H), 7.77-7.63 (m, 2H), 7.59-7.49 (m, 1H), 7.34 (td, J=4.5, 2.7 Hz, 2H), 7.16 (d, J=1.5 Hz, 1H), 3.90 (s, 3H), 2.78 (d, J=4.5 Hz, 3H). ¹³C NMR: (101 MHz, DMSO) δ 164.63, 164.34, 159.44, 155.02, 149.23, 148.02, 147.42, 146.48, 140.90, 137.72, 122.69, 121.72, 120.72, 119.92, 119.16, 118.11, 117.88, 116.26, 110.90, 53.69, 26.78.

### Example 30

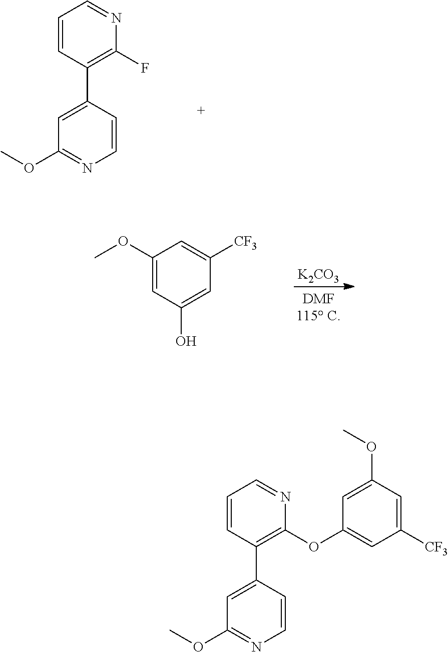

2′-methoxy-2-(3-methoxy-5-(trifluoromethyl)phenoxy)-3,4′-bipyridine (Compound 30): 2-fluoro-2′-methoxy-3,4′-bipyridine (102 mg, 0.5 mmol, 1.0 eq), 3-methoxy-5-(trifluoromethyl)phenol (96 mg, 0.5 mmol, 1.0 eq) and K₂CO₃ (276 mg, 2 mmol, 4.0 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product as a white solid. (117 mg, yield=62.2%, purity=95.5%)

TLC R_{f}=0.2 (PE/EA=10/1)

MS (ESI⁺): m/z=377.40 (M+1)

¹H NMR: (400 MHz, DMSO-d6) δ 8.26 (d, J=5.3 Hz, 1H), 8.21 (dd, J=4.9, 1.9 Hz, 1H), 8.05 (dd, J=7.5, 1.9 Hz, 1H), 7.38-7.28 (m, 2H), 7.17 (t, J=1.6 Hz, 2H), 7.12 (q, J=2.0, 1.6 Hz, 2H), 3.89 (s, 3H), 3.83 (s, 3H).

### Example 31

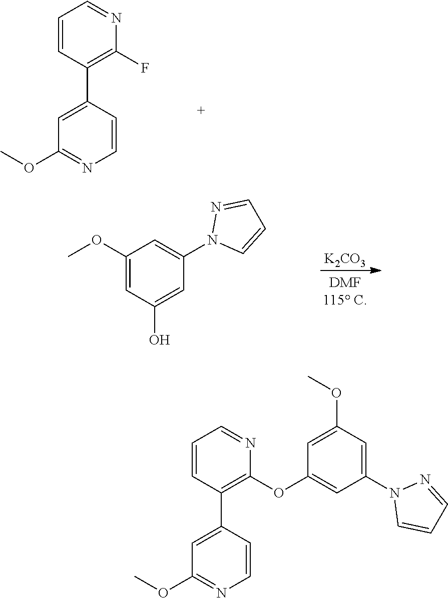

2′-methoxy-2-(3-methoxy-5-(1H-pyrazol-1-yl)phenoxy)-3,4′-bipyridine (Compound 31): 2-fluoro-2′-methoxy-3,4′-bipyridine (102 mg, 0.5 mmol, 1.0 eq), 3-methoxy-5-(1H-pyrazol-1-yl)phenol (95 mg, 0.5 mmol, 1 eq) and K₂CO₃ (276 mg, 2.0 mmol, 4 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product. (181 mg, yield=96.79%, purity=99%)

TLC R_{f}=0.4 (PE/EA=2/1)

MS (ESI⁺): m/z=375.60 (M+1)

¹H NMR: (400 MHz, DMSO-d6) δ 8.55 (d, J=2.6 Hz, 1H), 8.27 (d, J=5.4 Hz, 1H), 8.22 (dd, J=4.9, 1.9 Hz, 1H), 8.06 (dd, J=7.5, 1.9 Hz, 1H), 7.73 (d, J=1.7 Hz, 1H), 7.41-7.29 (m, 3H), 7.27 (t, J=2.0 Hz, 1H), 7.17 (d, J=1.4 Hz, 1H), 6.73 (t, J=2.2 Hz, 1H), 6.54 (t, J=2.2 Hz, 1H), 3.90 (s, 3H), 3.83 (s, 3H).

### Example 32

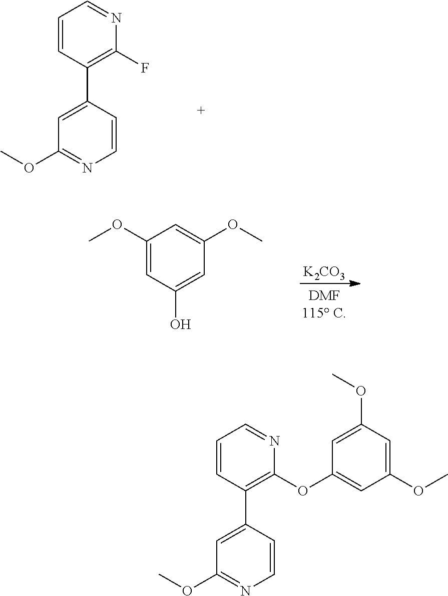

2-(3,5-dimethoxyphenoxy)-2′-methoxy-3,4′-bipyridine (Compound 32): 2-fluoro-2′-methoxy-3,4′-bipyridine (102 mg, 0.5 mmol, 1.0 eq), 3,5-dimethoxyphenol (77 mg, 0.5 mmol, 1.0 eq) and K₂CO₃ (138 mg, 1.0 mmol, 2 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product as a white solid. (92 mg, yield=54.4%, purity=99.5%)

TLC R_{f}=0.2 (PE/EA=10/1)

MS (ESI⁺): m/z=339.60 (M+1)

### Example 35

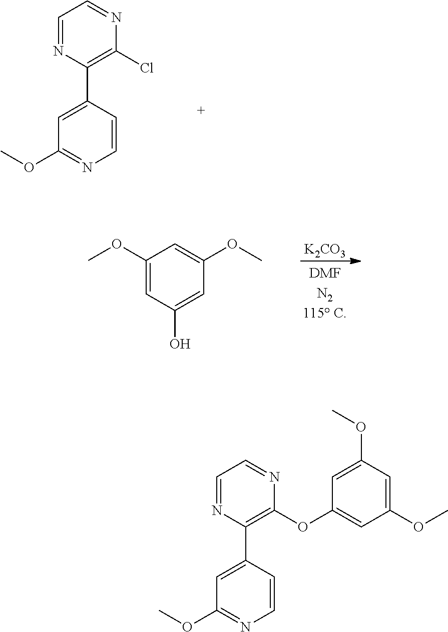

2-(3,5-dimethoxyphenoxy)-3-(2-methoxypyridin-4-yl)pyrazine (Compound 35): 2-chloro-3-(2-methoxypyridin-4-yl)pyrazine (110.5 mg, 0.5 mmol, 1.0 eq), 3,5-dimethoxyphenol (92.4 mg, 0.5 mmol, 1.0 eq) and K₂CO₃ (276 mg, 2 mmol, 4.0 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product as a white solid. (178.9 mg, yield>99%, purity=98.5%)

TLC R_{f}=0.3 (PE/EA=4/1)

MS (ESI⁺): m/z=340.40 (M+1)

¹H NMR: (400 MHz, DMSO-d6) δ 8.52 (d, J=2.5 Hz, 1H), 8.32 (dd, J=5.4, 0.7 Hz, 1H), 8.27 (d, J=2.5 Hz, 1H), 7.64 (dd, J=5.4, 1.5 Hz, 1H), 7.47 (dd, J=1.5, 0.7 Hz, 1H), 6.47 (d, J=2.3 Hz, 2H), 6.41 (t, J=2.2 Hz, 1H), 3.90 (s, 3H), 3.70 (d, J=13.9 Hz, 6H). ¹³C NMR: (101 MHz, DMSO) δ 163.89, 161.10, 159.09, 157.32, 154.46, 147.08, 145.36, 141.75, 139.72, 138.92, 116.51, 110.07, 100.17, 97.49, 93.89, 91.42, 55.43, 54.85, 53.32.

### Example 36

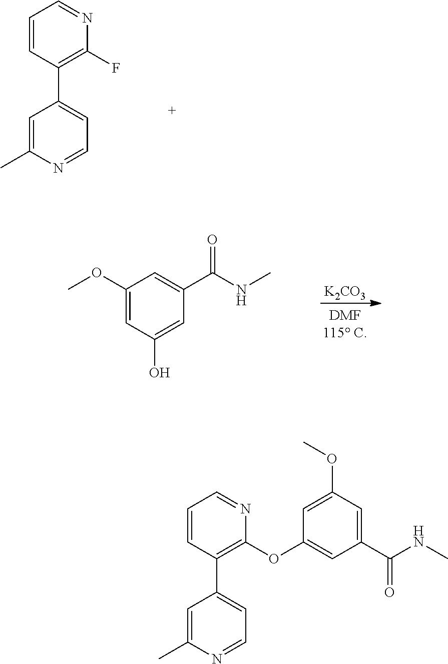

3-methoxy-N-methyl-5-((2′-methyl-[3,4′-bipyridin]-2-yl)oxy)benzamide (Compound 36): 2-fluoro-2′-methyl-3,4′-bipyridine (94 mg, 0.5 mmol, 1.0 eq), 3-hydroxy-5-methoxy-N-methylbenzamide (91 mg, 0.5 mmol, 1 eq) and K₂CO₃ (276 mg, 2.0 mmol, 4 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product. (179 mg, yield>99%, purity=92.83%)

TLC R_{f}=0.15 (PE/EA=1/4)

MS (ESI⁺): m/z=350.40 (M+1)

¹H NMR: (400 MHz, DMSO-d6) δ 8.54 (d, J=5.2 Hz, 1H), 8.45 (d, J=4.6 Hz, 1H), 8.20 (dd, J=4.9, 1.9 Hz, 1H), 8.03 (dd, J=7.5, 1.9 Hz, 1H), 7.57 (d, J=1.7 Hz, 1H), 7.53 (dd, J=5.2, 1.7 Hz, 1H), 7.36-7.23 (m, 2H), 7.19 (t, J=1.7 Hz, 1H), 6.95 (t, J=2.2 Hz, 1H), 3.80 (s, 3H), 2.77 (d, J=4.5 Hz, 3H), 2.54 (s, 3H). ¹³C NMR: (101 MHz, DMSO) δ 165.99, 160.69, 159.86, 158.61, 155.14, 149.44, 147.98, 143.95, 140.65, 137.05, 123.38, 122.86, 121.41, 120.31, 112.94, 110.82, 109.42, 56.10, 26.73, 24.61.

### Example 37

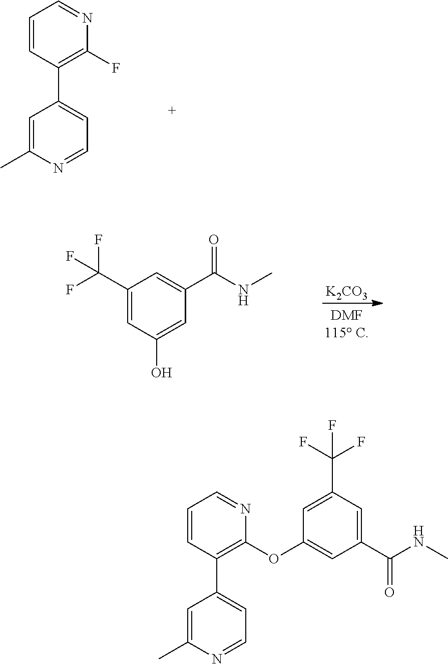

N-methyl-3-((2′-methyl-[3,4′-bipyridin]-2-yl)oxy)-5-(trifluoromethyl)benzamide (Compound 37): 2-fluoro-2′-methyl-3,4′-bipyridine (94 mg, 0.5 mmol, 1.0 eq), 3-hydroxy-N-methyl-5-(trifluoromethyl)benzamide (110 mg, 0.5 mmol, 1 eq) and K₂CO₃ (276 mg, 2.0 mmol, 4 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product. (154 mg, yield=79.59%, purity=99%)

TLC R_{f}=0.3 (PE/EA=1/4)

MS (ESI⁺): m/z=388.40 (M+1)

¹H NMR: (400 MHz, DMSO-d6) δ 8.72 (d, J=5.1 Hz, 1H), 8.55 (d, J=5.2 Hz, 1H), 8.21 (dd, J=4.8, 1.9 Hz, 1H), 8.08 (dd, J=7.4, 1.9 Hz, 1H), 8.05 (s, 1H), 7.93 (t, J=1.9 Hz, 1H), 7.85 (s, 1H), 7.62 (s, 1H), 7.58 (dd, J=5.2, 1.7 Hz, 1H), 7.35 (dd, J=7.5, 4.9 Hz, 1H), 2.80 (d, J=4.5 Hz, 3H), 2.55 (s, 3H).

### Example 38

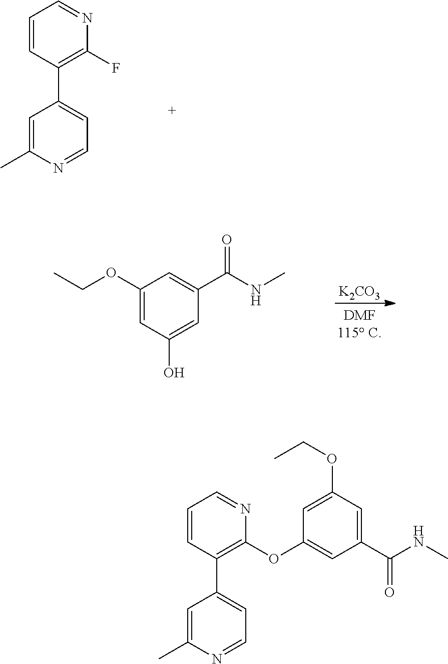

3-ethoxy-N-methyl-5-((2′-methyl-[3,4′-bipyridin]-2-yl)oxy)benzamide (Compound 38): 2-fluoro-2′-methyl-3,4′-bipyridine (94 mg, 0.5 mmol, 1.0 eq), 3-ethoxy-5-hydroxy-N-methylbenzamide (97.5 mg, 0.5 mmol, 1.0 eq) and K₂CO₃ (276 mg, 2 mmol, 4.0 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product. (144 mg, yield=79.3%, purity=99%)

TLC R_{f}=0.1 (PE/EA=1/4)

MS (ESI⁺): m/z=364.40 (M+1)

¹H NMR: (400 MHz, DMSO-d6) δ 8.53 (d, J=5.2 Hz, 1H), 8.44 (d, J=4.7 Hz, 1H), 8.20 (dd, J=4.9, 1.9 Hz, 1H), 8.03 (dd, J=7.5, 1.9 Hz, 1H), 7.57 (d, J=1.7 Hz, 1H), 7.52 (dd, J=5.2, 1.6 Hz, 1H), 7.30 (dd, J=7.5, 4.9 Hz, 1H), 7.26 (t, J=1.8 Hz, 1H), 7.18 (t, J=1.7 Hz, 1H), 6.92 (t, J=2.2 Hz, 1H), 4.11-4.03 (m, 2H), 2.76 (d, J=4.5 Hz, 3H), 2.54 (s, 3H), 1.33 (t, J=6.9 Hz, 3H).

¹³C NMR: (101 MHz, DMSO) δ 166.01, 159.92, 159.85, 158.60, 155.16, 149.44, 147.98, 143.95, 140.64, 137.02, 123.38, 122.90, 121.40, 120.31, 112.78, 111.17, 109.81, 64.08, 26.72, 24.61, 14.99.

### Example 39

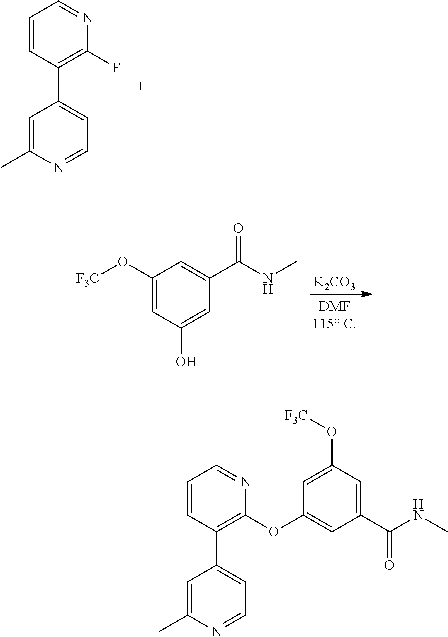

N-methyl-3-((2′-methyl-[3,4′-bipyridin]-2-yl)oxy)-5-(trifluoromethoxy)benzamide (Compound 39): 2-fluoro-2′-methyl-3,4′-bipyridine (94 mg, 0.5 mmol, 1.0 eq), 3-hydroxy-N-methyl-5-(trifluoromethoxy)benzamide (117.5 mg, 0.5 mmol, 1.0 eq) and K₂CO₃ (276 mg, 2 mmol, 4.0 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product as a white solid. (152.8 mg, yield=75.8%, purity=99.5%)

TLC R_{f}=0.3 (PE/EA=1/2)

MS (ESI⁺): m/z=404.30 (M+1)

¹H NMR: (400 MHz, DMSO-d6) δ 8.63 (q, J=4.5 Hz, 1H), 8.54 (d, J=5.2 Hz, 1H), 8.22 (dd, J=4.9, 1.9 Hz, 1H), 8.07 (dd, J=7.5, 1.9 Hz, 1H), 7.68 (dt, J=8.7, 1.5 Hz, 2H), 7.59 (d, J=1.6 Hz, 1H), 7.57-7.50 (m, 2H), 7.35 (dd, J=7.5, 4.8 Hz, 1H), 2.78 (d, J=4.5 Hz, 3H), 2.54 (s, 3H).

### Example 40

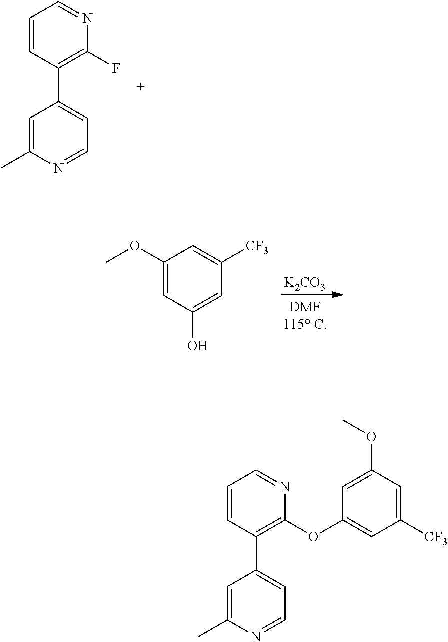

2-(3-methoxy-5-(trifluoromethyl)phenoxy)-2′-methyl-3,4′-bipyridine (Compound 40): 2-fluoro-2′-methyl-3,4′-bipyridine (94 mg, 0.5 mmol, 1.0 eq), 3-methoxy-5-(trifluoromethyl)phenol (96 mg, 0.5 mmol, 1 eq) and K₂CO₃ (276 mg, 2.0 mmol, 4 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product. (143 mg, yield=79.44%, purity=84.09%)

TLC R_{f}=0.5 (PE/EA=1/2)

MS (ESI⁺): m/z=361.40 (M+1)

### Example 41

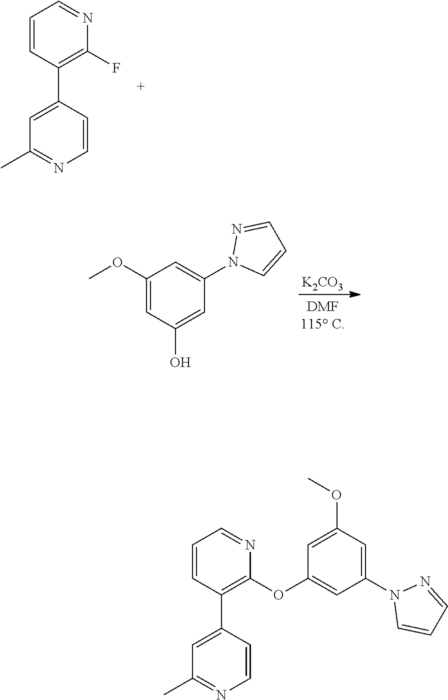

2-(3-methoxy-5-(1H-pyrazol-1-yl)phenoxy)-2′-methyl-3,4′-bipyridine (Compound 41): 2-fluoro-2′-methyl-3,4′-bipyridine (94 mg, 0.5 mmol, 1.0 eq), 3-methoxy-5-(1H-pyrazol-1-yl)phenol (95 mg, 0.5 mmol, 1 eq) and K₂CO₃ (276 mg, 2.0 mmol, 4 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product. (139 mg, yield=77.65%, purity=98.80%)

TLC R_{f}=0.4 (PE/EA=1/2)

MS (ESI⁺): m/z=359.50 (M+1)

¹H NMR: (400 MHz, DMSO-d6) δ 8.61-8.48 (m, 2H), 8.22 (dd, J=4.9, 1.9 Hz, 1H), 8.04 (dd, J=7.5, 1.9 Hz, 1H), 7.73 (d, J=1.7 Hz, 1H), 7.59 (d, J=1.7 Hz, 1H), 7.55 (dd, J=5.2, 1.7 Hz, 1H), 7.32 (td, J=6.0, 5.3, 3.5 Hz, 2H), 7.28 (t, J=2.0 Hz, 1H), 6.73 (t, J=2.1 Hz, 1H), 6.54 (t, J=2.1 Hz, 1H), 3.83 (s, 3H), 2.54 (s, 3H).

### Example 42

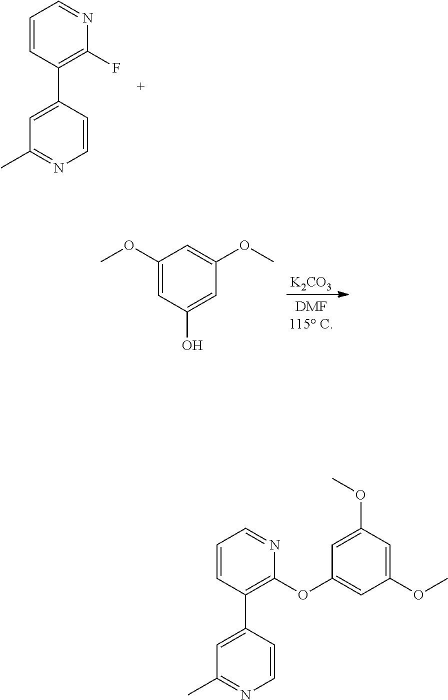

2-(3,5-dimethoxyphenoxy)-2′-methyl-3,4′-bipyridine (Compound 42): 2-fluoro-2′-methyl-3,4′-bipyridine (94 mg, 0.5 mmol, 1.0 eq), 3,5-dimethoxyphenol (92.4 mg, 0.5 mmol, 1.0 eq) and K₂CO₃ (276 mg, 2 mmol, 4.0 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product as a white solid. (165.6 mg, yield=99%, purity=97.1%)

TLC R_{f}=0.3 (PE/EA=1/1)

MS (ESI⁺): m/z=323.40 (M+1)

¹H NMR: (400 MHz, DMSO-d6) δ 8.52 (dd, J=5.2, 0.8 Hz, 1H), 8.20 (dd, J=4.9, 1.9 Hz, 1H), 7.99 (dd, J=7.5, 1.9 Hz, 1H), 7.56-7.52 (m, 1H), 7.50 (dd, J=5.2, 1.8 Hz, 1H), 7.27 (dd, J=7.5, 4.9 Hz, 1H), 6.35 (t, J=2.2 Hz, 1H), 6.33 (d, J=2.2 Hz, 2H), 3.71 (s, 6H), 2.52 (s, 3H). ¹³C NNMR: (101 MHz, DMSO) b 160.98, 159.39, 158.06, 155.53, 148.91, 147.51, 143.52, 140.02, 122.87, 122.41, 120.90, 119.67, 99.96, 96.70, 55.35, 24.11.

### Example 43

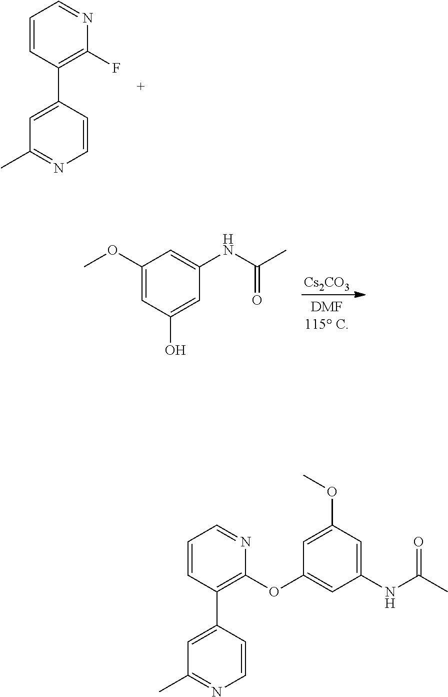

N-(3-methoxy-5-((2′-methyl-[3,4′-bipyridin]-2-yl)oxy)phenyl)acetamide (Compound 43): 2-fluoro-2′-methyl-3,4′-bipyridine (94 mg, 0.5 mmol, 1.0 eq), N-(3-hydroxy-5-methoxyphenyl)acetamide (91 mg, 0.5 mmol, leg) and Cs₂C₀₃ (326 mg, 1.0 mmol, 2 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product. (134 mg, yield=77.01%, purity=99%)

TLC R_{f}=0.25 (PE/EA=1/4)

MS (ESI⁺): m/z=350.60 (M+1)

¹H NMR: (400 MHz, DMSO-d6) δ 9.98 (s, 1H), 8.53 (d, J=5.1 Hz, 1H), 8.22 (dd, J=4.8, 1.9 Hz, 1H), 8.03 (dd, J=7.5, 1.9 Hz, 1H), 7.54 (d, J=1.6 Hz, 1H), 7.49 (dd, J=5.1, 1.7 Hz, 1H), 7.30 (dd, J=7.5, 4.9 Hz, 1H), 7.04 (t, J=2.1 Hz, 1H), 6.98 (t, J=1.9 Hz, 1H), 6.45 (t, J=2.2 Hz, 1H), 3.71 (s, 3H), 2.53 (s, 3H), 2.02 (s, 3H).

### Example 44

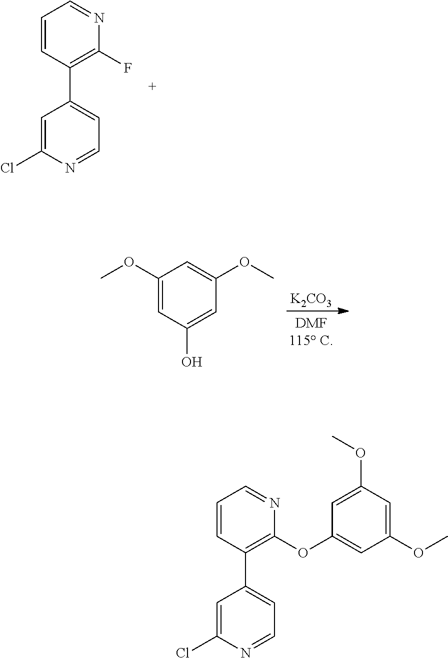

2′-chloro-2-(3,5-dimethoxyphenoxy)-3,4′-bipyridine (Compound 44): 2′-chloro-2-fluoro-3,4′-bipyridine (104 mg, 0.5 mmol, 1.0 eq), 3,5-dimethoxyphenol (77 mg, 0.5 mmol, 1.0 eq) and K₂CO₃ (276 mg, 2 mmol, 4.0 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product. (161.4 mg, yield=94.3%, purity=99.0%)

TLC R_{f}=0.25 (PE/EA=4/1)

MS (ESI⁺): m/z=343.60 (M+1)

### Example 45

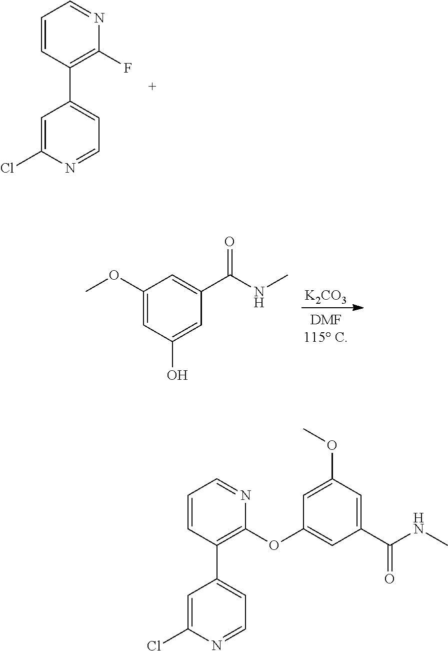

3-((2′-chloro-[3,4′-bipyridin]-2-yl)oxy)-5-methoxy-N-methylbenzamide (Compound 45): 2′-chloro-2-fluoro-3,4′-bipyridine (104 mg, 0.5 mmol, 1.0 eq), 3-hydroxy-5-methoxy-N-methylbenzamide (90.5 mg, 0.5 mmol, 1.0 eq) and K₂CO₃ (276 mg, 2 mmol, 4.0 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product. (200 mg, yield=100%, purity=98.9%)

TLC R_{f}=0.2 (PE/EA=1/2)

MS (ESI⁺): m/z=370.50 (M+1)

### Example 46

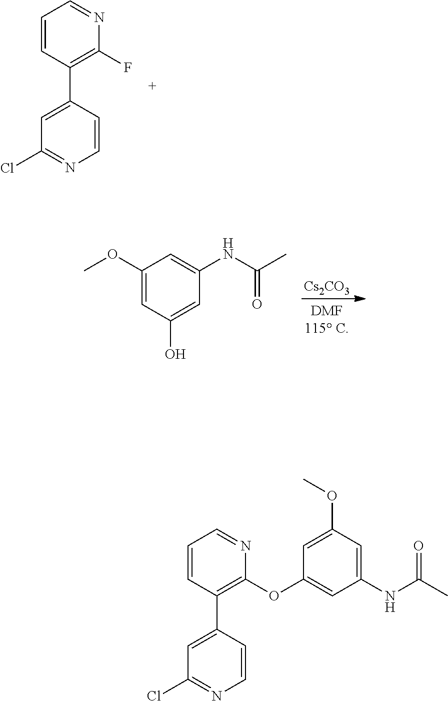

N-(3-((2′-chloro-[3,4′-bipyridin]-2-yl)oxy)-5-methoxyphenyl)acetamide (Compound 46): 2′-chloro-2-fluoro-3,4′-bipyridine (105 mg, 0.5 mmol, 1.0 eq), N-(3-hydroxy-5-methoxyphenyl)acetamide (91 mg, 0.5 mmol, 1 eq) and Cs₂CO₃ (326 mg, 1.0 mmol, 2 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product. (146 mg, yield=78.92%, purity=99%)

TLC R_{f}=0.2 (PE/EA=1/2)

MS (ESI⁺): m/z=370.50 (M+1)

¹H NMR: (400 MHz, DMSO-d6) δ 10.00 (s, 1H), 8.52 (d, J=5.2 Hz, 1H), 8.25 (dd, J=4.9, 1.9 Hz, 1H), 8.11 (dd, J=7.5, 1.9 Hz, 1H), 7.84 (d, J=1.4 Hz, 1H), 7.75 (dd, J=5.1, 1.5 Hz, 1H), 7.32 (dd, J=7.5, 4.8 Hz, 1H), 7.02 (dt, J=13.0, 2.0 Hz, 2H), 6.49 (t, J=2.2 Hz, 1H), 3.72 (s, 3H), 2.02 (s, 3H).

### Example 47

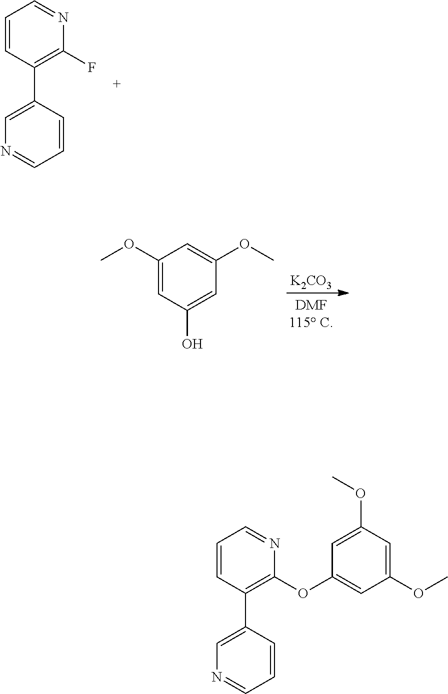

2-(3,5-dimethoxyphenoxy)-3,3′-bipyridine (Compound 47): 2-fluoro-3,3′-bipyridine (87 mg, 0.5 mmol, 1.0 eq), 3,5-dimethoxyphenol (115.5 mg, 0.75 mmol, 1.5 eq) and K₂CO₃ (138 mg, 1.0 mmol, 2 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product as a colorless oil. (43 mg, yield=27.9%, purity=89.4%)

TLC R_{f}=0.25 (PE/EA=2/1)

MS (ESI⁺): m/z=309.40 (M+1)

### Example 50

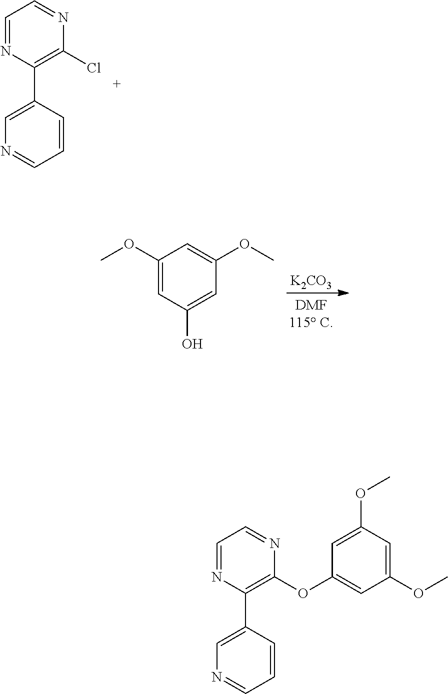

2-(3,5-dimethoxyphenoxy)-3,3′-bipyridine (Compound 50): 2-chloro-3-(pyridin-3-yl)pyrazine (115 mg, 0.6 mmol, 1.0 eq), 3,5-dimethoxyphenol (93 mg, 0.6 mmol, 1.0 eq) and K₂CO₃ (166 mg, 1.0 mmol, 2 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 8 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product as a white solid. (166.8 mg, yield=89.9%, purity=98%)

TLC R_{f}=0.22 (PE/EA=2/1)

MS (ESI⁺): m/z=310.50 (M+1)

¹H NMR: ¹H NMR (400 MHz, DMSO-d6) δ 9.24 (dt, J=2.6, 1.2 Hz, 1H), 8.68 (dd, J=4.8, 1.5 Hz, 1H), 8.51 (tt, J=3.3, 2.0 Hz, 1H), 8.43 (ddd, J=8.0, 2.3, 1.7 Hz, 1H), 8.29-8.17 (m, 1H), 7.64-7.48 (m, 1H), 6.55-6.45 (m, 2H), 6.41 (t, J=2.2 Hz, 1H), 3.73 (s, 6H). ¹³C NMR: (101 MHz, DMSO) δ 161.08, 157.20, 154.53, 150.03, 149.58, 140.76, 140.36, 138.95, 136.32, 131.02, 123.36, 100.19, 97.43, 55.42

### Example 52

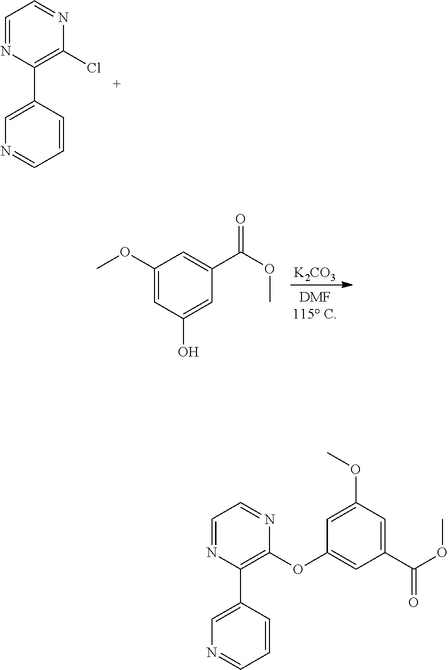

methyl 3-methoxy-5-((3-(pyridin-3-yl)pyrazin-2-yl)oxy)benzoate (Compound 52): 2-chloro-3-(pyridin-3-yl)pyrazine (115 mg, 0.6 mmol, 1.0 eq), methyl 3-hydroxy-5-methoxybenzoate (110 mg, 0.6 mmol, 1.0 eq) and K₂CO₃ (166 mg, 1.0 mmol, 2 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 8 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product as a white solid. (150 mg, yield=74%, purity=98.8%)

TLC R_{f}=0.2 (PE/EA=2/1)

MS (ESI⁺): m/z=338.5 (M+1)

¹H NMR: ¹H NMR (400 MHz, DMSO-d6) δ 9.25 (dd, J=2.3, 0.9 Hz, 1H), 8.68 (dd, J=4.8, 1.7 Hz, 1H), 8.53 (d, J=2.6 Hz, 1H), 8.46 (ddd, J=8.0, 2.3, 1.7 Hz, 1H), 8.22 (d, J=2.6 Hz, 1H), 7.57 (ddd, J=8.0, 4.8, 0.9 Hz, 1H), 7.43 (dd, J=2.1, 1.4 Hz, 1H), 7.36 (dd, J=2.4, 1.4 Hz, 1H), 7.24 (t, J=2.3 Hz, 1H), 3.83 (d, J=9.8 Hz, 6H). ¹³C NMR: δ 165.35, 160.44, 157.06, 153.79, 150.07, 149.63, 140.53, 140.48, 139.14, 136.42, 131.78, 130.94, 123.36, 114.80, 112.94, 111.33, 55.80, 52.38.

### Example 55

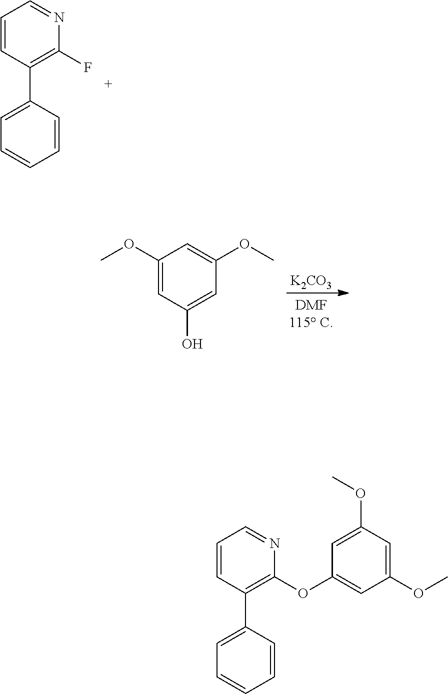

2-(3, 5-dimethoxyphenoxy)-3-phenylpyridine (Compound 55): 2-fluoro-3-phenylpyridine (103.8 mg, 0.6 mmol, 1.0 eq), 3,5-dimethoxyphenol (123 mg, 0.8 mmol, 1.33 eq) and K₂CO₃ (166 mg, 1.2 mmol, 2 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 8 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product as a white solid. (137 mg, yield=89.2%, purity=95.5%)

TLC R_{f}=0.3 (PE/EA=10/1)

MS (ESI⁺): m/z=308.60 (M+1)

### Example 56

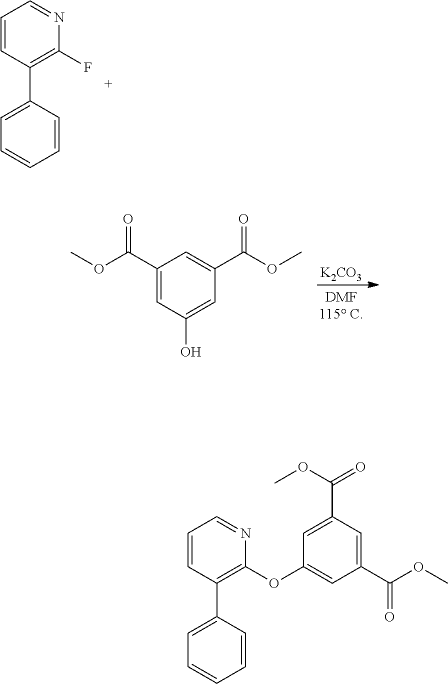

dimethyl 5-((3-phenylpyridin-2-yl)oxy)isophthalate (Compound 56): 2-fluoro-3-phenylpyridine (103.8 mg, 0.6 mmol, 1.0 eq), 3,5-dimethoxyphenol (126 mg, 0.6 mmol, 1.33 eq) and K₂CO₃ (166 mg, 1.0 mmol, 2 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 8 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product as a white solid. (62 mg, yield=28.4%, purity=97%)

TLC R_{f}=0.45 (PE/EA=4/1)

MS (ESI⁺): m/z=364.40 (M+1)

¹H NMR: (400 MHz, DMSO-d6) δ 8.30 (t, J=1.5 Hz, 1H), 8.13 (dd, J=4.9, 1.9 Hz, 1H), 7.97 (dd, J=7.4, 1.9 Hz, 1H), 7.92 (d, J=1.5 Hz, 2H), 7.75-7.66 (m, 2H), 7.52-7.44 (m, 2H), 7.44-7.36 (m, 1H), 7.30 (dd, J=7.5, 4.8 Hz, 1H), 3.87 (s, 6H). ¹³C NMR: (101 MHz, DMSO) δ 164.77, 158.91, 154.25, 146.03, 140.40, 135.34, 131.63, 129.10, 128.38, 127.90, 126.39, 125.41, 125.07, 120.31, 52.60, 39.48.

### Example 57

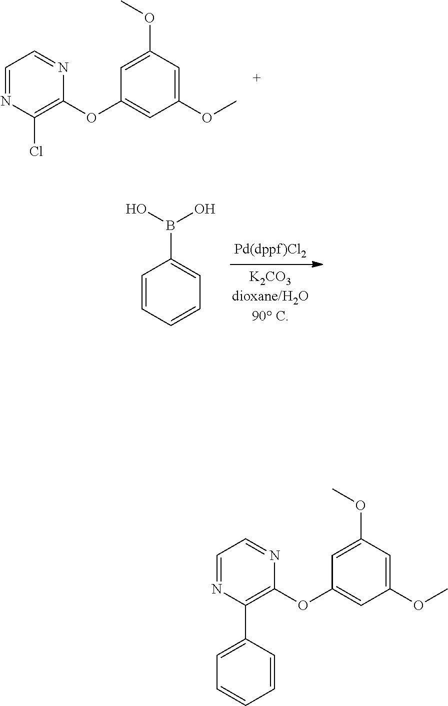 [SMILES: COc1cc(OC)cc(Oc2nccnc2Cl)c1 ; OB(O)c1ccccc1 ; COc1cc(OC)cc(Oc2nccnc2-c2ccccc2)c1]

2-(3,5-dimethoxyphenoxy)-3-phenylpyrazine (Compound 57): 2-chloro-3-(3,5-dimethoxyphenoxy)pyrazine (160 mg, 0.6 mmol, 1.0 eq), phenylboronic acid (110 mg, 0.9 mmol, 1.5 eq), Pd(dppf)Cl₂ (11 mg, 0.015 mmol, 2.5% eq) and K₂CO₃ (273 mg, 1.98 mmol, 3.3 eq) were added to a round-bottom flask with a magnetic bar, then 5 ml dioxane and 0.6 ml H₂O were added as solvent. The reaction vessel was evacuated and backfilled with N₂ three times and protected with a balloon of N₂. The reaction mixture was heated at 90° C. for at least 12 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product as a white solid. (156 mg, yield=99.1%, purity=95.6%)

TLC R_{f}=0.20 (PE/EA=10/1)

MS (ESI⁺): m/z=309.40 (M+1)

### Example 58

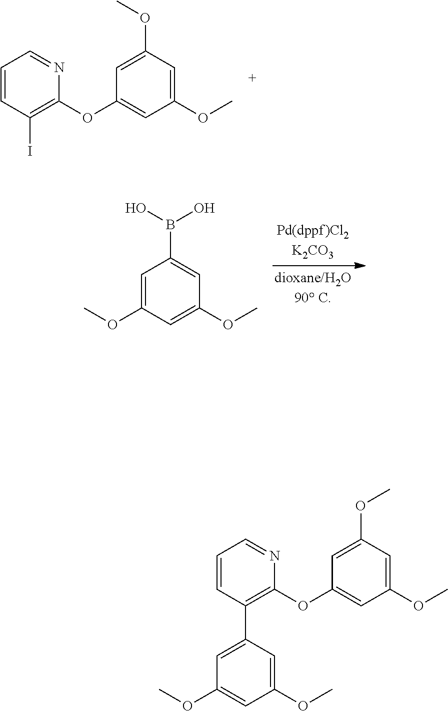

2-(3,5-dimethoxyphenoxy)-3-(3,5-dimethoxyphenyl)pyridine (Compound 58): 2-(3,5-dimethoxyphenoxy)-3-iodopyridine (178 mg, 0.5 mmol, 1.0 eq), 3,5-dimethoxyphenylboronic acid (135.8 mg, 0.6 mmol, 1.2 eq), Pd(dppf)Cl₂ (9.1 mg, 0.0125 mmol, 0.025 eq) and K₂CO₃ (227.7 mg, 1.65 mmol, 3.3 eq) were added to a round-bottom flask with a magnetic bar. The flask was purged with N₂. Then 3 ml dioxane and 0.375 ml water were added as solvent. The reaction vessel was evacuated and backfilled with N₂ three times and protected with a balloon of N₂. The reaction mixture was heated at 90° C. for 8 h with vigorous stirring. The cooled solution was diluted with 30 ml ethyl acetate and washed with brine. The organic phase was dried over anhydrous Na₂SO₄ and concentrated in vacuo. The residue was purified by silica gel flash chromatography to afford the product as a white solid. (170 mg, yield=92.5%, purity=98.7%)

TLC R_{f}=0.22 (PE/EA=8/1)

MS (ESI⁺): m/z=368.40 (M+1)

### Example 59

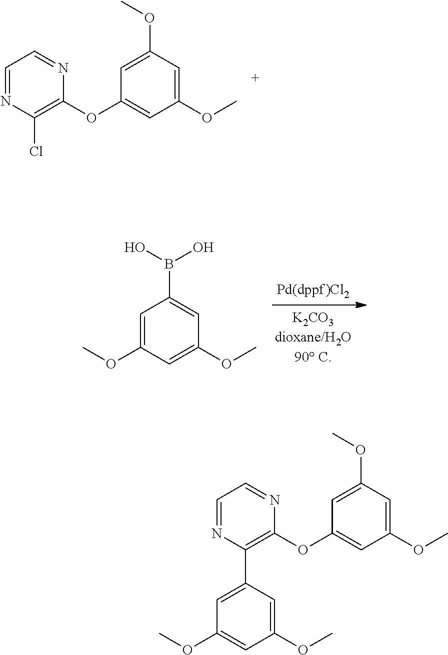

2-(3,5-dimethoxyphenoxy)-3-(3,5-dimethoxyphenyl)pyrazine (Compound 59): 2-chloro-3-(3,5-dimethoxyphenoxy)pyrazine (160 mg, 0.6 mmol, 1.0 eq), 3,5-dimethoxyphenylboronic acid (164 mg, 0.9 mmol, 1.5 eq), Pd(dppf)Cl₂ (11 mg, 0.015 mmol, 2.5% eq) and K₂CO₃ (273 mg, 1.98 mmol, 3.3 eq) were added to a round-bottom flask with a magnetic bar, then 5 ml dioxane and 0.6 ml H₂O were added as solvent. The reaction vessel was evacuated and backfilled with N₂ three times and protected with a balloon of N₂. The reaction mixture was heated at 90° C. for at least 12 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product as a white solid. (186 mg, yield=84.2%, purity=98.5%)

TLC R_{f}=0.23 (PE/EA=4/1)

MS (ESI⁺): m/z=369.50 (M+1)

### Example 60

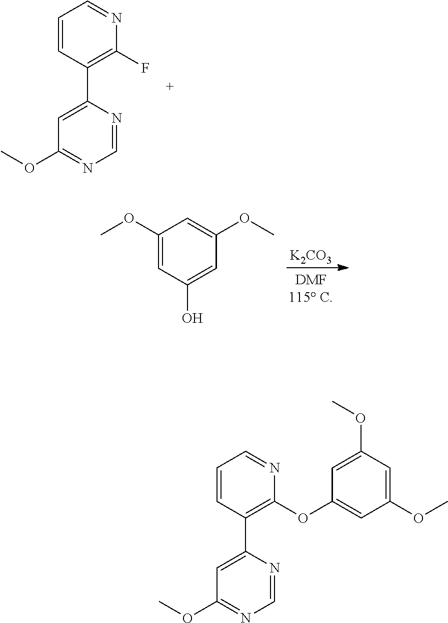

4-(2-(3,5-dimethoxyphenoxy)pyridin-3-yl)-6-methoxypyrimidine (Compound 60): 4-(2-fluoropyridin-3-yl)-6-methoxypyrimidine (102.5 mg, 0.5 mmol, 1.0 eq), 3,5-dimethoxyphenol (92.4 mg, 0.5 mmol, 1.0 eq) and K₂CO₃ (276 mg, 2 mmol, 4.0 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product as a white solid. (143.2 mg, yield=84.4%, purity=97.6%)

TLC R_{f}=0.35 (PE/EA=4/1)

MS (ESI⁺): m/z=340.40 (M+1)

¹H NMR: (400 MHz, DMSO-d6) δ 8.92 (d, J=1.1 Hz, 1H), 8.48 (dd, J=7.6, 2.0 Hz, 1H), 8.25 (dd, J=4.8, 2.0 Hz, 1H), 7.55 (d, J=1.1 Hz, 1H), 7.31 (dd, J=7.6, 4.8 Hz, 1H), 6.38 (s, 3H), 3.96 (s, 3H), 3.72 (s, 6H). ¹³C NMR: (101 MHz, DMSO) δ 169.54, 161.00, 160.18, 160.09, 158.19, 155.10, 148.88, 140.21, 120.58, 119.50, 107.42, 100.14, 97.00, 55.37, 53.89.

### Example 61

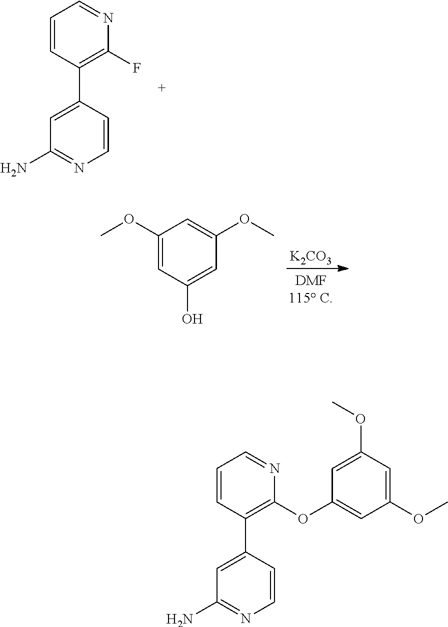

2-(3,5-dimethoxyphenoxy)-[3,4′-bipyridin]-2′-amine (Compound 61): 2-fluoro-[3,4′-bipyridin]-2′-amine (94.5 mg, 0.5 mmol, 1.0 eq), 3,5-dimethoxyphenol (92.4 mg, 0.6 mmol, 1.2 eq) and K₂CO₃ (276 mg, 2 mmol, 4.0 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product as a white solid. (129.5 mg, yield=80.0%, purity=91.5%)

TLC R_{f}=0.1 (PE/EA=1/2)

MS (ESI⁺): m/z=324.50 (M+1)

### Example 62

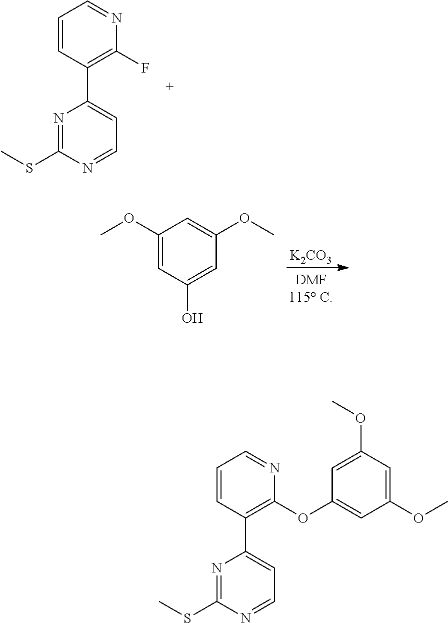

4-(2-(3,5-dimethoxyphenoxy)pyridin-3-yl)-2-(methylthio)pyrimidine (Compound 62): 4-(2-fluoropyridin-3-yl)-2-(methylthio)pyrimidine (110.5 mg, 0.5 mmol, 1.0 eq), 3,5-dimethoxyphenol (77 mg, 0.6 mmol, 1.2 eq) and K₂CO₃ (276 mg, 2 mmol, 4.0 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product. (195.9 mg, yield=99%, purity=98.7%)

TLC R_{f}=0.45 (PE/EA=4/1)

MS (ESI⁺): m/z=356.40 (M+1)

### Example 63

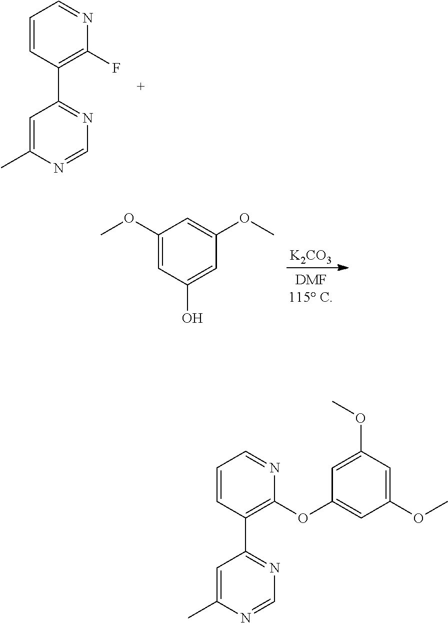

4-(2-(3,5-dimethoxyphenoxy)pyridin-3-yl)-6-methylpyrimidine (Compound 63): 4-(2-fluoropyridin-3-yl)-6-methylpyrimidine (83.7 mg, 0.44 mmol, 1.0 eq), 3,5-dimethoxyphenol (81.6 mg, 0.53 mmol, 1.2 eq) and K₂CO₃ (276 mg, 2 mmol, 4.0 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product. (131.8 mg, yield=92.7%, purity=99.5%)

TLC R_{f}=0.25 (PE/EA=2/1)

MS (ESI⁺): m/z=324.40 (M+1)

### Example 64

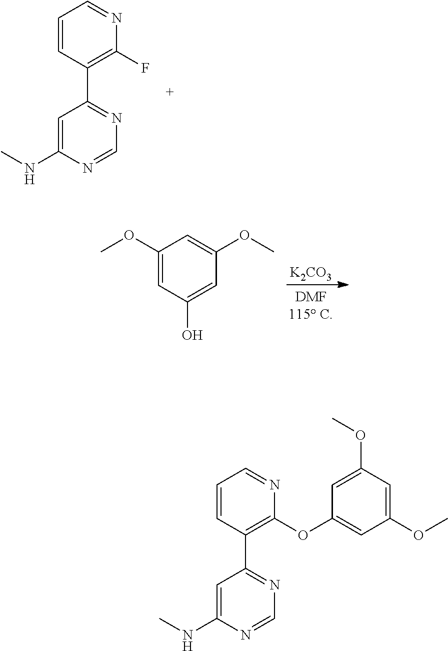

6-(2-(3,5-dimethoxyphenoxy)pyridin-3-yl)-N-methylpyrimidin-4-amine (Compound 64): 6-(2-fluoropyridin-3-yl)-N-methylpyrimidin-4-amine (87 mg, 0.4 mmol, 1.0 eq), 3,5-dimethoxyphenol (77 mg, 0.5 mmol, 1.2 eq) and K₂CO₃ (276 mg, 2 mmol, 5.0 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product as a white solid. (134.3 mg, yield=99.3%, purity=97.9%)

TLC R_{f}=0.4 (PE/EA=1/4)

MS (ESI⁺): m/z=339.60 (M+1)

### Example 65

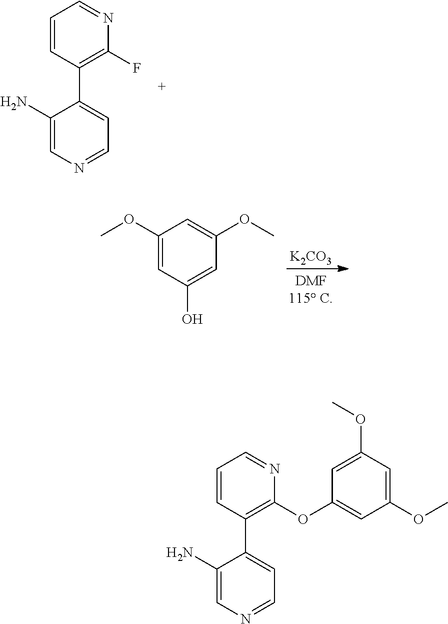

2-(3,5-dimethoxyphenoxy)-[3,4′-bipyridin]-3′-amine (Compound 65): 2-fluoro-[3,4′-bipyridin]-3′-amine (94.5 mg, 0.5 mmol, 1.0 eq), 3,5-dimethoxyphenol (92.4 mg, 0.6 mmol, 1.2 eq) and K₂CO₃ (276 mg, 2 mmol, 4.0 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product as a white solid. (119.4 mg, yield=73.9%, purity=45.6%)

TLC R_{f}=0.25 (PE/EA=1/4)

MS (ESI⁺): m/z=324.50 (M+1)

### Example 66

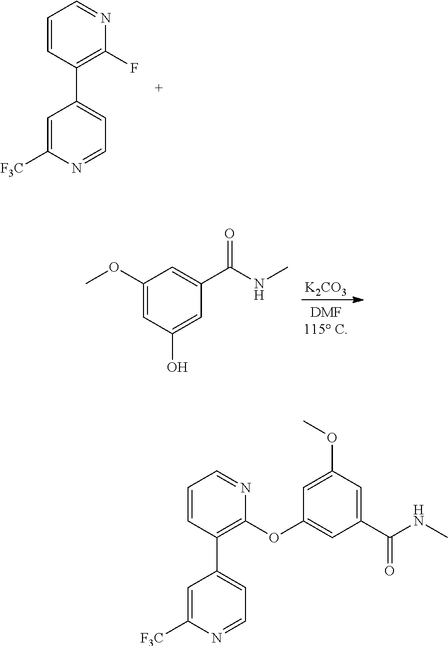

3-methoxy-N-methyl-5-((2′-(trifluoromethyl)-[3,4′-bipyridin]-2-yl)oxy)benzamide (Compound 66): 2-fluoro-2′-(trifluoromethyl)-3,4′-bipyridine (121 mg, 0.5 mmol, 1.0 eq), 3-hydroxy-5-methoxy-N-methylbenzamide (90.5 mg, 0.5 mmol, 1.0 eq) and K₂CO₃ (276 mg, 2 mmol, 4.0 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product. (171.5 mg, yield=85.1%, purity=97.9%)

TLC R_{f}=0.25 (PE/EA=1/2)

MS (ESI⁺): m/z=404.30 (M+1)

¹H NMR: (400 MHz, DMSO-d6) δ 8.89 (d, J=5.1 Hz, 1H), 8.45 (q, J=4.5 Hz, 1H), 8.30-8.22 (m, 2H), 8.19 (dd, J=7.5, 1.9 Hz, 1H), 8.10 (dd, J=5.1, 1.6 Hz, 1H), 7.35 (dd, J=7.5, 4.9 Hz, 1H), 7.29 (dd, J=2.5, 1.4 Hz, 1H), 7.24 (d, J=1.7 Hz, 1H), 7.01 (t, J=2.2 Hz, 1H), 3.81 (s, 3H), 2.77 (d, J=4.5 Hz, 3H). ¹³C NMR: (101 MHz, DMSO) δ 165.98, 160.70, 159.85, 154.84, 150.88, 148.83, 145.97, 141.10, 137.12, 127.72, 121.25, 121.07, 121.04, 120.33, 113.14, 110.99, 109.61, 56.10, 26.71.

### Example 67

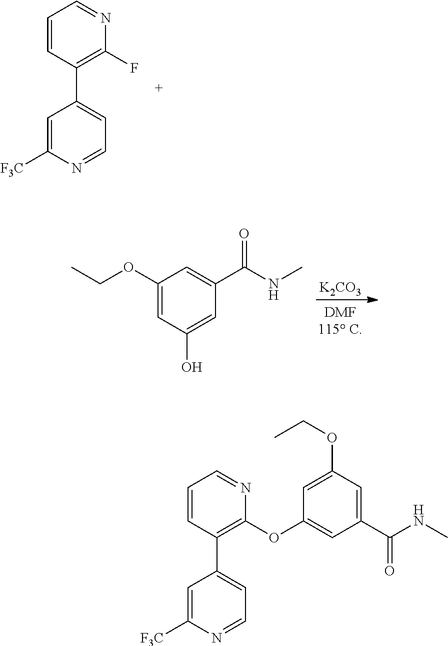

3-ethoxy-N-methyl-5-((2′-(trifluoromethyl)-[3,4′-bipyridin]-2-yl)oxy)benzamide (Compound 67): 2-fluoro-2′-(trifluoromethyl)-3,4′-bipyridine (73 mg, 0.3 mmol, 1.0 eq), 3-ethoxy-5-hydroxy-N-methylbenzamide (58.5 mg, 0.3 mmol, 1.0 eq) and K₂CO₃ (166 mg, 1.2 mmol, 4.0 eq) were added to a round-bottom flask with a magnetic bar, then 2 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product as a white solid. (104.9 mg, yield=83.8%, purity=99.1%)

TLC R_{f}=0.25 (PE/EA=1/2)

MS (ESI⁺): m/z=418.30 (M+1)

¹H NMR: (400 MHz, DMSO-d6) δ 8.89 (d, J=5.1 Hz, 1H), 8.43 (q, J=4.4 Hz, 1H), 8.30-8.23 (m, 2H), 8.19 (dd, J=7.5, 1.9 Hz, 1H), 8.10 (dd, J=5.1, 1.6 Hz, 1H), 7.35 (dd, J=7.5, 4.9 Hz, 1H), 7.30-7.25 (m, 1H), 7.21 (t, J=1.8 Hz, 1H), 6.98 (t, J=2.2 Hz, 1H), 4.07 (q, J=7.0 Hz, 2H), 2.76 (d, J=4.5 Hz, 3H), 1.33 (t, J=7.0 Hz, 3H). ¹³C NMR: (101 MHz, DMSO) δ 165.98, 159.92, 159.84, 154.85, 150.89, 148.84, 145.98, 141.11, 137.07, 127.73, 121.28, 121.08, 121.05, 120.34, 112.97, 111.34, 110.01, 64.09, 26.72, 14.97.

### Example 81

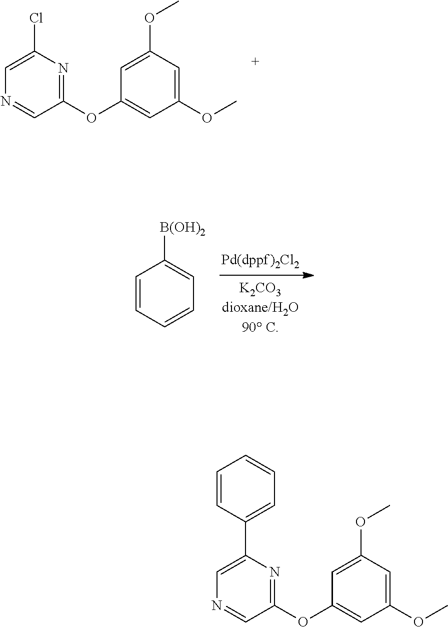 [SMILES: COc1cc(OC)cc(Oc2cncc(Cl)n2)c1 ; OB(O)c1ccccc1 ; COc1cc(OC)cc(Oc2cncc(-c3ccccc3)n2)c1]

2-(3,5-dimethoxyphenoxy)-6-phenylpyrazine (Compound 81): 2-chloro-6-(3,5-dimethoxyphenoxy)pyrazine (133.4 mg, 0.5 mmol, 1.0 eq), phenylboronic acid (73.2 mg, 0.6 mmol, 1.2 eq), Pd(dppf)C₁₂ (9.1 mg, 0.0125 mmol, 0.025 eq) and K₂CO₃ (227.7 mg, 1.65 mmol, 3.3 eq) were added to a round-bottom flask with a magnetic bar. The flask was purged with N₂. Then 3 ml dioxane and 0.375 ml water were added as solvent. The reaction vessel was evacuated and backfilled with N₂ three times and protected with a balloon of N₂. The reaction mixture was heated at 90° C. for 8 h with vigorous stirring. The cooled solution was diluted with 30 ml ethyl acetate and washed with brine. The organic phase was dried over anhydrous Na₂SO₄, and concentrated in vacuo. The residue was purified by silica gel flash chromatography to afford the product as a white solid. (148 mg, yield=96%, purity>99%)

TLC R_{f}=0.33 (PE/EA=10/1)

MS (ESI⁺): m/z=309.40 (M+1)

### Example 82

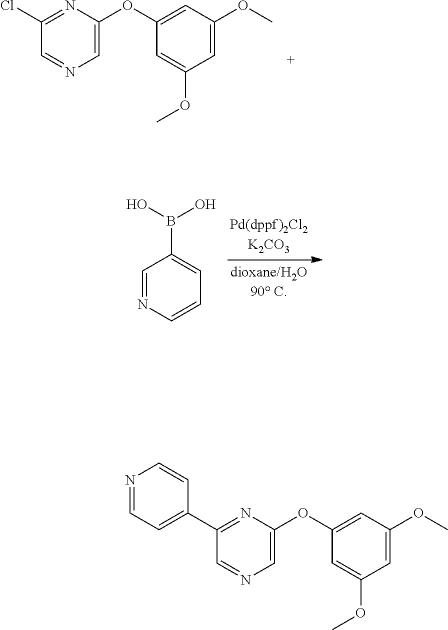 [SMILES: COc1cc(OC)cc(Oc2cncc(Cl)n2)c1 ; OB(O)c1cccnc1 ; COc1cc(OC)cc(Oc2cncc(-c3ccncc3)n2)c1]

2-(3,5-dimethoxyphenoxy)-6-(pyridin-3-yl)pyrazine (Compound 82): 2-chloro-6-(3,5-dimethoxyphenoxy)pyrazine (133.4 mg, 0.5 mmol, 1.0 eq), pyridin-3-ylboronic acid (73.8 mg, 0.6 mmol, 1.2 eq), Pd(dppf)C₁₂ (9.1 mg, 0.0125 mmol, 0.025 eq) and K₂CO₃ (227.7 mg, 1.65 mmol, 3.3 eq) were added to a round-bottom flask with a magnetic bar. The flask was purged with N₂. Then 3 ml dioxane and 0.375 ml water were added as solvent. The reaction vessel was evacuated and backfilled with N₂ three times and protected with a balloon of N₂. The reaction mixture was heated at 90° C. for 8 h with vigorous stirring. The cooled solution was diluted with 30 ml ethyl acetate and washed with brine. The organic phase was dried over anhydrous Na₂SO₄, and concentrated in vacuo. The residue was purified by silica gel flash chromatography to afford the product as a white solid. (64 mg, yield=41.4%, purity=99.5%)

TLC R_{f}=0.20 (PE/EA=2/1)

MS (ESI⁺): m/z=310.50 (M+1)

### Example 83

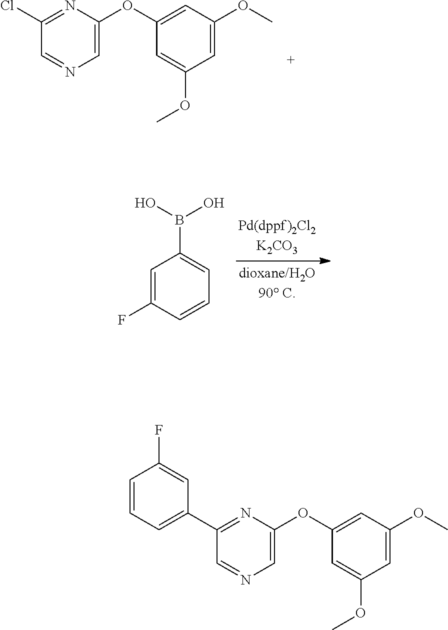 [SMILES: COc1cc(OC)cc(Oc2cncc(Cl)n2)c1 ; OB(O)c1cccc(F)c1 ; COc1cc(OC)cc(Oc2cncc(-c3cccc(F)c3)n2)c1]

2-(3,5-dimethoxyphenoxy)-6-(3-fluorophenyl)pyrazine (Compound 83): 2-chloro-6-(3,5-dimethoxyphenoxy)pyrazine (133.4 mg, 0.5 mmol, 1.0 eq), (3-fluorophenyl)boronic acid (105 mg, 0.75 mmol, 1.5 eq), Pd(dppf)Cl₂ (9.1 mg, 0.0125 mmol, 0.025 eq) and K₂CO₃ (227.7 mg, 1.65 mmol, 3.3 eq) were added to a round-bottom flask with a magnetic bar. The flask was purged with N₂. Then 3 ml dioxane and 0.375 ml water were added as solvent. The reaction vessel was evacuated and backfilled with N₂ three times and protected with a balloon of N₂. The reaction mixture was heated at 90° C. for 8 h with vigorous stirring. The cooled solution was diluted with 30 ml ethyl acetate and washed with brine. The organic phase was dried over anhydrous Na₂SO₄, and concentrated in vacuo. The residue was purified by silica gel flash chromatography to afford the product as a white solid. (134 mg, yield=82.1%, purity=98.1%)

TLC R_{f}=0.33 (PE/EA=8/1)

MS (ESI⁺): m/z=327.50 (M+1)

### Example 84

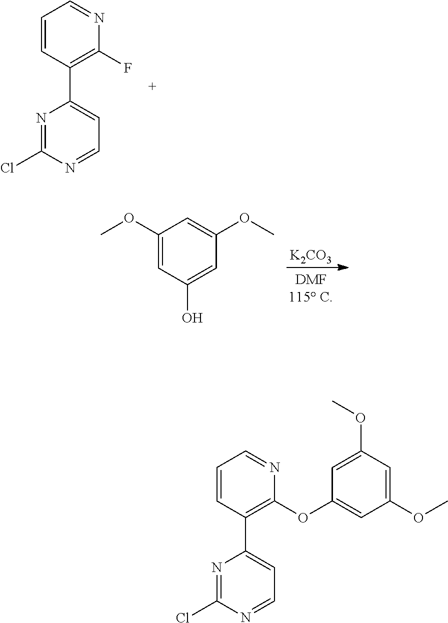

2-chloro-4-(2-(3,5-dimethoxyphenoxy)pyridin-3-yl)pyrimidine (Compound 84): 2-chloro-4-(2-fluoropyridin-3-yl)pyrimidine (108.5 mg, 0.5 mmol, 1.0 eq), 3,5-dimethoxyphenol (77 mg, 0.5 mmol, 1.0 eq) and K₂CO₃ (276 mg, 2 mmol, 4.0 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product. (109.2 mg, yield=63.7%, purity=88.9%)

TLC R_{f}=0.23 (PE/EA=2/1)

MS (ESI⁺): m/z=344.40 (M+1)

### Example 85

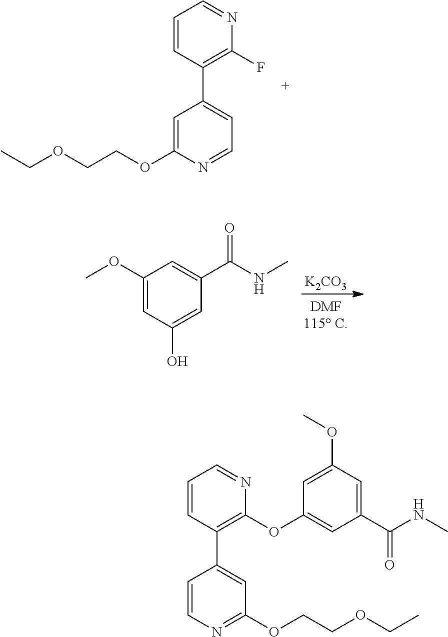

3-((2′-(2-ethoxyethoxy)-[3,4′-bipyridin]-2-yl)oxy)-5-methoxy-N-methylbenzamide (Compound 85): 2′-(2-ethoxyethoxy)-2-fluoro-3,4′-bipyridine (131.2 mg, 0.5 mmol, 1.0 eq), 3-hydroxy-5-methoxy-N-methylbenzamide (91 mg, 0.5 mmol, 1.0 eq) and K₂CO₃ (276 mg, 2 mmol, 4.0 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product. (197 mg, yield=93%, purity=94.88%)

TLC R_{f}=0.35 (PE/EA=1/4)

MS (ESI⁺): m/z=424.60 (M+1)

### Example 86

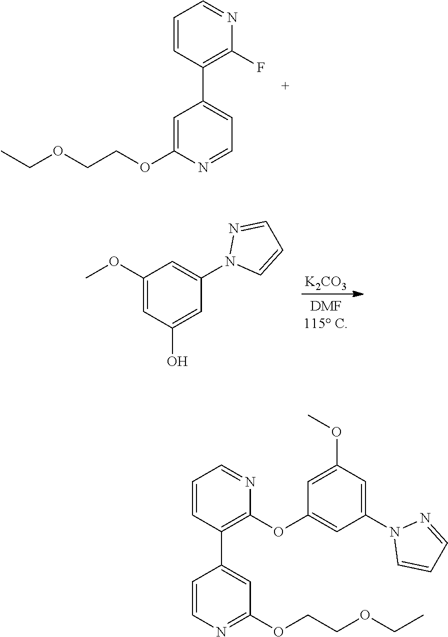

2′-(2-ethoxyethoxy)-2-(3-methoxy-5-(1H-pyrazol-1-yl)phenoxy)-3,4′-bipyridine (Compound 86): 2′-(2-ethoxyethoxy)-2-fluoro-3,4′-bipyridine (131.2 mg, 0.5 mmol, 1.0 eq), 3-methoxy-5-(1H-pyrazol-1-yl)phenol (95 mg, 0.5 mmol, 1.0 eg) and K₂CO₃ (276 mg, 2 mmol, 4.0 eq) were added to a round-bottom flask with a magnetic bar, then 3 ml DMF was added as solvent.

The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product. (126 mg, yield=58.2%, purity=98.6%)

TLC R_{f}=0.30 (PE/EA=2/1)

MS (ESI⁺): m/z=433.40 (M+1)

### Example 87

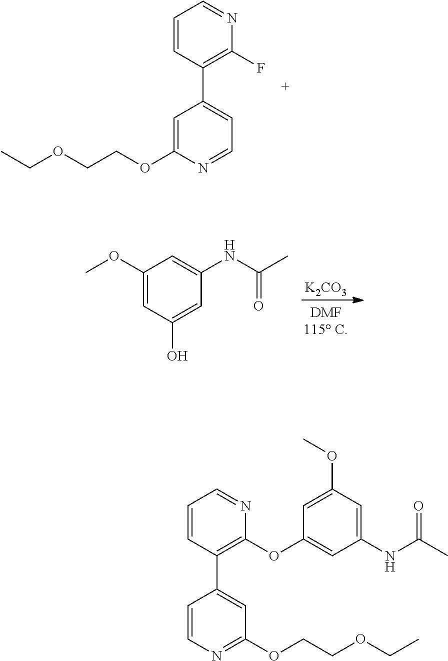

N-(3-((2′-(2-ethoxyethoxy)-[3,4′-bipyridin]-2-yl)oxy)-5-methoxyphenyl)acetamide (Compound 87): 2′-(2-ethoxyethoxy)-2-fluoro-3,4′-bipyridine (131.2 mg, 0.5 mmol, 1.0 eq), N-(3-hydroxy-5-methoxyphenyl)acetamide (91 mg, 0.5 mmol, 1.0 eq) and K₂CO₃ (276 mg, 2 mmol, 4.0 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product. (155.2 mg, yield=73.4%, purity=98.4%)

TLC R_{f}=0.3 (PE/EA=1/2)

MS (ESI⁺): m/z=424.60 (M+1)

¹H NMR: (400 MHz, DMSO-d6) δ 10.01 (s, 1H), 8.33-8.21 (m, 2H), 8.10 (dd, J=7.5, 1.9 Hz, 1H), 7.41-7.29 (m, 2H), 7.16 (d, J=1.4 Hz, 1H), 7.09 (t, J=2.1 Hz, 1H), 7.01 (t, J=1.9 Hz, 1H), 6.50 (t, J=2.2 Hz, 1H), 4.55-4.35 (m, 2H), 3.81-3.69 (m, 5H), 3.54 (q, J=7.0 Hz, 2H), 2.06 (s, 3H), 1.17 (t, J=7.0 Hz, 3H).

### Example 88

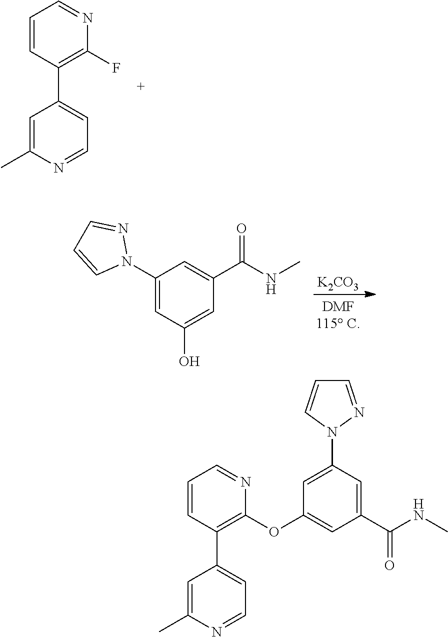

N-methyl-3-((2′-methyl-[3,4′-bipyridin]-2-yl)oxy)-5-(1H-pyrazol-1-yl)benzamide (Compound 88): 2-fluoro-2′-methyl-3,4′-bipyridine (94 mg, 0.5 mmol, 1.0 eq), 3-hydroxy-N-methyl-5-(1H-pyrazol-1-yl)benzamide (109 mg, 0.5 mmol, 1 eq) and K₂CO₃ (276 mg, 2.0 mmol, 4 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product. (168 mg, yield=87.27%, purity=99%)

TLC R_{f}=0.10 (PE/EA=1/2)

MS (ESI⁺): m/z=386.20 (M+1)

¹H NMR: (400 MHz, DMSO-d6) δ 8.64 (dd, J=6.9, 3.5 Hz, 2H), 8.61 (d, J=5.2 Hz, 1H), 8.27 (dd, J=4.9, 1.9 Hz, 1H), 8.24 (t, J=1.7 Hz, 1H), 8.13 (dd, J=7.5, 1.9 Hz, 1H), 7.91 (t, J=2.1 Hz, 1H), 7.84 (d, J=1.7 Hz, 1H), 7.66 (d, J=1.6 Hz, 1H), 7.62 (dd, J=5.3, 1.7 Hz, 1H), 7.59 (t, J=1.8 Hz, 1H), 7.39 (dd, J=7.5, 4.9 Hz, 1H), 6.64 (t, J=2.1 Hz, 1H), 2.86 (d, J=4.5 Hz, 3H), 2.60 (s, 3H).

### Example 89

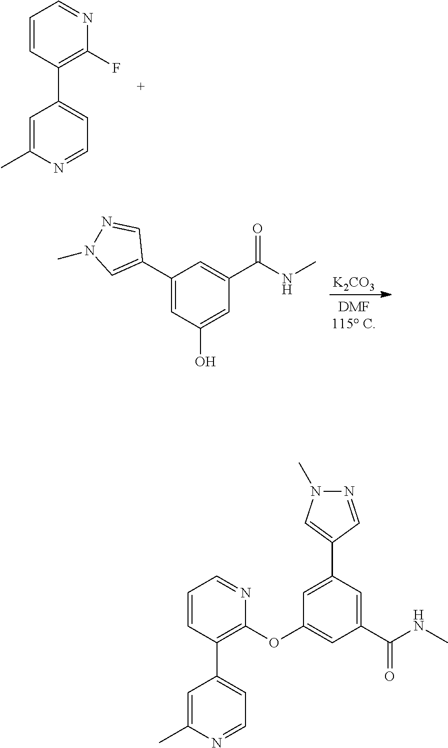

N-methyl-3-(1-methyl-1H-pyrazol-4-yl)-5-((2′-methyl-[3,4′-bipyridin]-2-yl)oxy)benzamide (Compound 89): 2-fluoro-2′-methyl-3,4′-bipyridine (94 mg, 0.5 mmol, 1.0 eq), 3-hydroxy-N-methyl-5-(1-methyl-1H-pyrazol-4-yl)benzamide (166 mg, 0.5 mmol, 1 eq) and K₂CO₃ (276 mg, 2.0 mmol, 4 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product. (330 mg, yield=99%, purity=95.14%)

TLC R_{f}=0.25 (DCM/MeOH=20/1)

MS (ESI⁺): m/z=400.40 (M+1)

¹H NMR: (400 MHz, DMSO-d6) δ 8.47 (d, J=5.2 Hz, 1H), 8.40 (q, J=4.5 Hz, 1H), 8.14 (s, 1H), 8.12 (dd, J=4.9, 1.9 Hz, 1H), 7.97 (dd, J=7.5, 1.9 Hz, 1H), 7.86 (d, J=0.8 Hz, 1H), 7.82 (t, J=1.6 Hz, 1H), 7.52 (d, J=1.7 Hz, 1H), 7.50-7.46 (m, 2H), 7.33 (t, J=1.9 Hz, 1H), 7.23 (dd, J=7.5, 4.9 Hz, 1H), 3.79 (s, 3H), 2.72 (d, J=4.5 Hz, 3H), 2.47 (s, 3H).

### Example 90

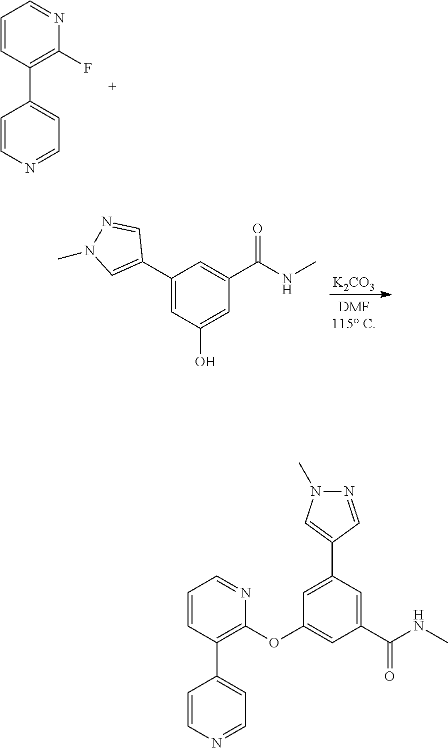

3-([3,4′-bipyridin]-2-yloxy)-N-methyl-5-(1-methyl-1H-pyrazol-4-yl)benzamide (Compound 90): 2-fluoro-3,4′-bipyridine (87 mg, 0.5 mmol, 1.0 eq), 3-hydroxy-N-methyl-5-(1-methyl-1H-pyrazol-4-yl)benzamide (166 mg, 0.5 mmol, 1 eq) and K₂CO₃ (276 mg, 2.0 mmol, 4 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product. (210 mg, yield=99%, purity=88.2%)

TLC R_{f}=0.25 (DCM/MeOH=20/1)

MS (ESI⁺): m/z=386.40 (M+1)

¹H NMR: (400 MHz, DMSO-d6) δ 8.73-8.63 (m, 2H), 8.47 (q, J=4.5 Hz, 1H), 8.25-8.17 (m, 2H), 8.12-8.05 (m, 1H), 7.93 (s, 1H), 7.90 (t, J=1.6 Hz, 1H), 7.79-7.71 (m, 2H), 7.57 (t, J=1.9 Hz, 1H), 7.41 (t, J=1.9 Hz, 1H), 7.32 (dd, J=7.5, 4.9 Hz, 1H), 3.87 (s, 3H), 2.80 (d, J=4.5 Hz, 3H).

### Example 91

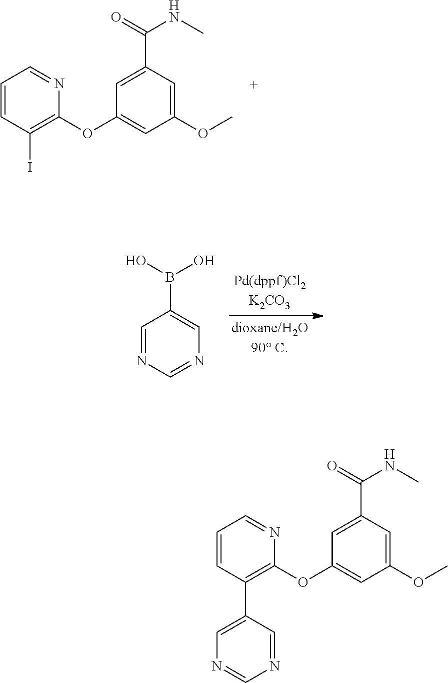 [SMILES: CNC(=O)c1cc(OC)cc(Oc2ncccc2I)c1 ; OB(O)c1cncnc1 ; CNC(=O)c1cc(OC)cc(Oc2ncccc2-c2cncnc2)c1]

3-methoxy-N-methyl-5-((3-(pyrimidin-5-yl)pyridin-2-yl)oxy)benzamide (Compound 91): 3-((3-iodopyridin-2-yl)oxy)-5-methoxy-N-methylbenzamide (192 mg, 0.5 mmol, 1.0 eq), pyrimidin-5-ylboronic acid (62 mg, 0.5 mmol, 1.0 eq), Pd(dppf)C₁₂ (9.1 mg, 0.0125 mmol, 0.025 eq) and K₂CO₃ (227.7 mg, 1.65 mmol, 3.3 eq) were added to a round-bottom flask with a magnetic bar. The flask was purged with N₂. Then 3 ml dioxane and 0.375 ml water were added as solvent. The reaction vessel was evacuated and backfilled with N₂ three times and protected with a balloon of N₂. The reaction mixture was heated at 90° C. for 8 h with vigorous stirring. The cooled solution was diluted with 30 ml ethyl acetate and washed with brine. The organic phase was dried over anhydrous Na₂SO₄ and concentrated in vacuo. The residue was purified by silica gel flash chromatography to afford the product as a white solid. (119.3 mg, yield=71%, purity=99%)

TLC R_{f}=0.20 (PE/EA=1/5)

MS (ESI⁺): m/z=337.20 (M+1)

¹H NMR: (400 MHz, DMSO-d6) δ 9.23 (s, 1H), 9.18 (s, 2H), 8.42 (d, J=4.6 Hz, 1H), 8.23 (dd, J=4.9, 1.9 Hz, 1H), 8.15 (dd, J=7.5, 1.9 Hz, 1H), 7.35 (dd, J=7.5, 4.9 Hz, 1H), 7.27 (dd, J=2.4, 1.4 Hz, 1H), 7.21 (t, J=1.8 Hz, 1H), 7.00 (t, J=2.2 Hz, 1H), 3.80 (s, 3H), 2.76 (d, J=4.5 Hz, 3H).

### Example 92

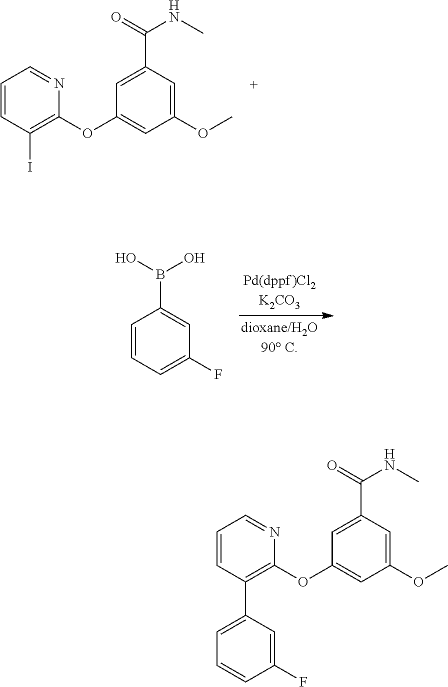 [SMILES: CNC(=O)c1cc(OC)cc(Oc2ncccc2I)c1 ; OB(O)c1cccc(F)c1 ; CNC(=O)c1cc(OC)cc(Oc2ncccc2-c2cccc(F)c2)c1]

3-((3-(3-fluorophenyl)pyridin-2-yl)oxy)-5-methoxy-N-methylbenzamide (Compound 92): 3-((3-iodopyridin-2-yl)oxy)-5-methoxy-N-methylbenzamide (192 mg, 0.5 mmol, 1.0 eq), (3-fluorophenyl)boronic acid (195 mg, 1.0 mmol, 2.0 eq), Pd(dppf)C₁₂ (9.1 mg, 0.0125 mmol, 0.025 eq) and K₂CO₃ (227.7 mg, 1.65 mmol, 3.3 eq) were added to a round-bottom flask with a magnetic bar. The flask was purged with N₂. Then 3 ml dioxane and 0.375 ml water were added as solvent. The reaction vessel was evacuated and backfilled with N₂ three times and protected with a balloon of N₂. The reaction mixture was heated at 90° C. for 8 h with vigorous stirring. The cooled solution was diluted with 30 ml ethyl acetate and washed with brine. The organic phase was dried over anhydrous Na₂SO₄ and concentrated in vacuo. The residue was purified by silica gel flash chromatography to afford the product as a white solid. (143 mg, yield=81.2%, purity=99%)

TLC R_{f}=0.25 (PE/EA=1/2)

MS (ESI⁺): m/z=353.10 (M+1)

¹H NMR: (400 MHz, DMSO-d6) δ 8.42 (d, J=4.6 Hz, 1H), 8.17 (dd, J=4.8, 1.9 Hz, 1H), 8.00 (dd, J=7.5, 1.9 Hz, 1H), 7.64-7.48 (m, 3H), 7.37-7.21 (m, 3H), 7.17 (t, J=1.8 Hz, 1H), 6.94 (t, J=2.2 Hz, 1H), 3.80 (s, 3H), 2.76 (d, J=4.5 Hz, 3H).

### Example 93

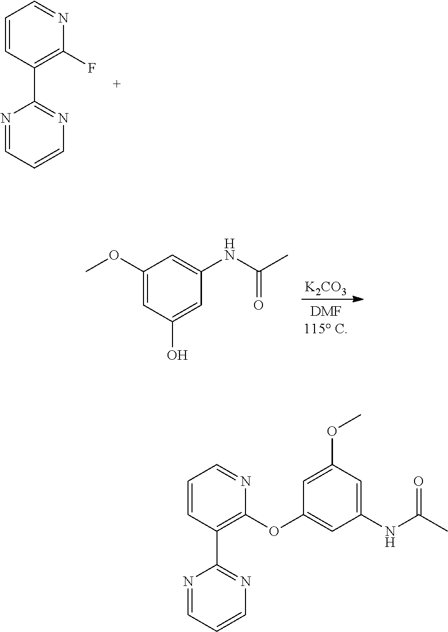

N-(3-methoxy-5-((3-(pyrimidin-2-yl)pyridin-2-yl)oxy)phenyl)acetamide (Compound 93): 2-(2-fluoropyridin-3-yl)pyrimidine (87 mg, 0.5 mmol, 1.0 eq), N-(3-hydroxy-5-methoxyphenyl)acetamide (90 mg, 0.5 mmol, 1 eq) and K₂CO₃ (276 mg, 2.0 mmol, 4 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product. (105.3 mg, yield=62.7%, purity=99%)

TLC R_{f}=0.25 (PE/EA=1/4)

MS (ESI⁺): m/z=337.10 (M+1)

¹H NMR: (400 MHz, DMSO-d6) δ 9.99 (s, 1H), 9.00 (d, J=4.9 Hz, 2H), 8.39-8.18 (m, 2H), 7.55 (t, J=4.9 Hz, 1H), 7.36 (dd, J=7.5, 4.9 Hz, 1H), 7.07 (t, J=2.1 Hz, 1H), 6.99 (t, J=1.9 Hz, 1H), 6.40 (t, J=2.2 Hz, 1H), 3.74 (s, 3H), 2.05 (s, 3H).

### Example 94

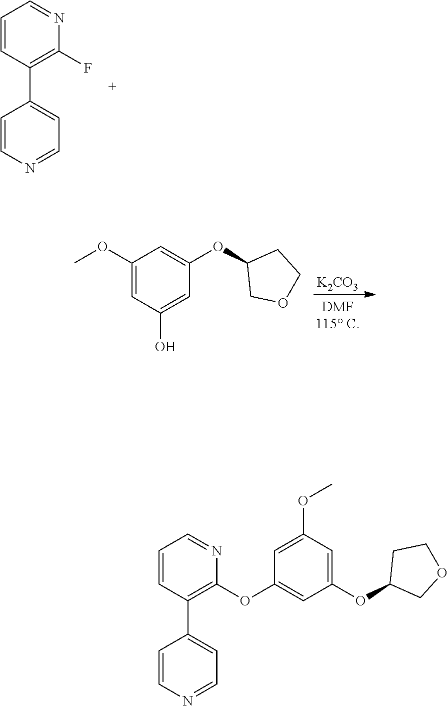

(S)-2-(3-methoxy-5-((tetrahydrofuran-3-yl)oxy)phenoxy)-3,4′-bipyridine (Compound 94): 2-fluoro-3,4′-bipyridine (87 mg, 0.5 mmol, 1.0 eq), 3-methoxy-5-((tetrahydrofuran-3-yl)oxy)phenol (168 mg, 0.8 mmol, 1.6 eq) and K₂CO₃ (276 mg, 2.0 mmol, 4 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product. (163 mg, yield=89.6%, purity=98.4%)

TLC R_{f}=0.3 (PE/EA=1/1)

MS (ESI⁺): m/z=365.10 (M+1)

¹H NMR: (400 MHz, DMSO-d6) δ 8.66-8.49 (m, 2H), 8.15 (dd, J=4.8, 1.9 Hz, 1H), 7.97 (dd, J=7.5, 1.9 Hz, 1H), 7.77-7.50 (m, 2H), 7.23 (dd, J=7.5, 4.9 Hz, 1H), 6.40-6.14 (m, 3H), 3.87-3.58 (m, 8H), 2.11 (dtd, J=14.3, 8.2, 6.2 Hz, 1H), 1.97-1.76 (m, 1H).

### Example 95

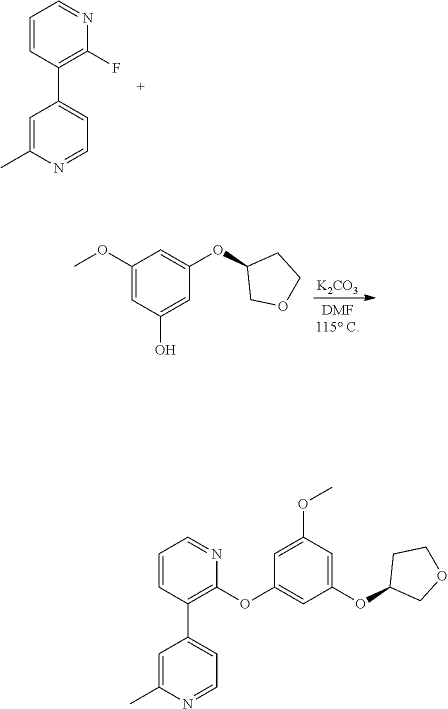

(S)-2-(3-methoxy-5-((tetrahydrofuran-3-yl)oxy)phenoxy)-2′-methyl-3,4′-bipyridine (Compound 95): 2-fluoro-2′-methyl-3,4′-bipyridine (94 mg, 0.5 mmol, 1.0 eq), 3-methoxy-5-((tetrahydrofuran-3-yl)oxy)phenol (168 mg, 0.8 mmol, 1.6 eq) and K₂CO₃ (276 mg, 2.0 mmol, 4 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product. (137 mg, yield=72.49%, purity=99.43%)

TLC R_{f}=0.2 (PE/EA=2/1)

MS (ESI⁺): m/z=379.00 (M+1)

¹H NMR: (400 MHz, DMSO-d6) δ 8.45 (d, J=5.2 Hz, 1H), 8.14 (dd, J=4.9, 1.9 Hz, 1H), 7.93 (dd, J=7.5, 1.9 Hz, 1H), 7.47 (d, J=1.7 Hz, 1H), 7.43 (dd, J=5.3, 1.7 Hz, 1H), 7.22 (dd, J=7.5, 4.9 Hz, 1H), 6.25 (dd, J=8.0, 2.1 Hz, 3H), 3.87-3.54 (m, 8H), 2.46 (s, 3H), 2.12 (ddd, J=13.2, 8.5, 6.0 Hz, 1H), 1.94-1.84 (m, 1H).

### Example 96

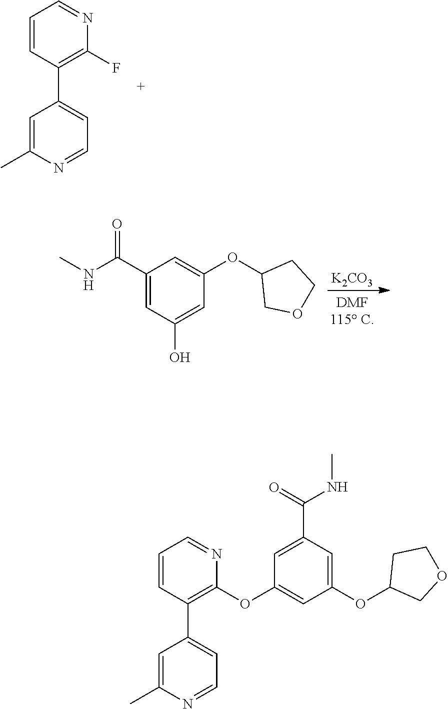

N-methyl-3-((2′-methyl-[3,4′-bipyridin]-2-yl)oxy)-5-((tetrahydrofuran-3-yl)oxy)benzamide (Compound 96): 2-fluoro-2′-methyl-3,4′-bipyridine (112.8 mg, 0.6 mmol, 1.2 eq), 3-hydroxy-N-methyl-5-((tetrahydrofuran-3-yl)oxy)benzamide (118.6 mg, 0.5 mmol, 1.0 eq) and K₂CO₃ (276 mg, 2.0 mmol, 4 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product. (173 mg, yield=85.3%, purity=98.61%)

TLC R_{f}=0.3 (DCM/MeOH=40/1)

MS (ESI⁺): m/z=406.20 (M+1)

¹H NMR: (400 MHz, DMSO-d6) δ 8.54 (d, J=5.2 Hz, 1H), 8.42 (q, J=4.5 Hz, 1H), 8.21 (dd, J=4.9, 1.9 Hz, 1H), 8.04 (dd, J=7.5, 1.9 Hz, 1H), 7.57 (d, J=1.7 Hz, 1H), 7.53 (dd, J=5.2, 1.7 Hz, 1H), 7.31 (dd, J=7.5, 4.9 Hz, 1H), 7.27-7.20 (m, 1H), 7.18 (t, J=1.8 Hz, 1H), 6.94 (t, J=2.2 Hz, 1H), 4.21-3.63 (m, 5H), 2.76 (d, J=4.5 Hz, 3H), 2.54 (s, 3H), 2.23 (dtd, J=14.1, 8.3, 6.1 Hz, 1H), 1.98-1.91 (m, 1H).

### Example 97

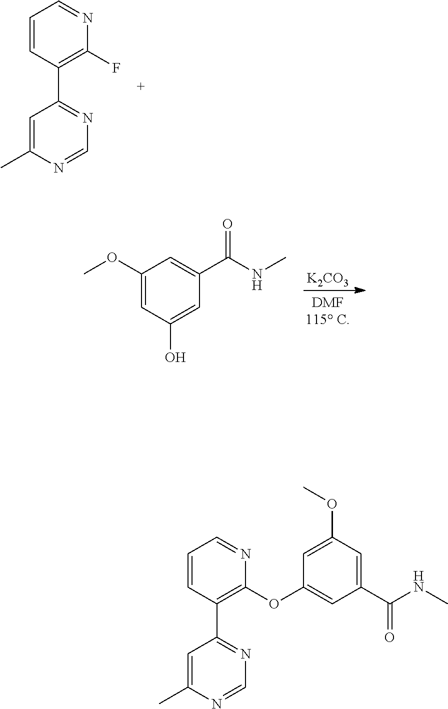

3-methoxy-N-methyl-5-((3-(6-methylpyrimidin-4-yl)pyridin-2-yl)oxy)benzamide (Compound 97): 4-(2-fluoropyridin-3-yl)-6-methylpyrimidine (95 mg, 0.5 mmol, 1.0 eq), 3-hydroxy-5-methoxy-N-methylbenzamide (91 mg, 0.5 mmol, 1 eq) and K₂CO₃ (276 mg, 2.0 mmol, 4 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product. (137 mg, yield=78.29%, purity=89.59%)

TLC R_{f}=0.2 (PE/EA=1/6)

MS (ESI⁺): m/z=350.90 (M+1)

¹H NMR: (400 MHz, DMSO-d6) δ 9.11 (d, J=1.3 Hz, 1H), 8.41 (dd, J=7.6, 2.0 Hz, 1H), 8.37 (q, J=4.6 Hz, 1H), 8.20 (dd, J=4.8, 2.0 Hz, 1H), 8.07-7.92 (m, 1H), 7.28 (dd, J=7.6, 4.8 Hz, 1H), 7.22 (dd, J=2.4, 1.4 Hz, 1H), 7.16 (t, J=1.7 Hz, 1H), 6.93 (t, J=2.2 Hz, 1H), 3.74 (s, 3H), 2.69 (d, J=4.5 Hz, 3H), 2.47 (s, 3H).

### Example 98

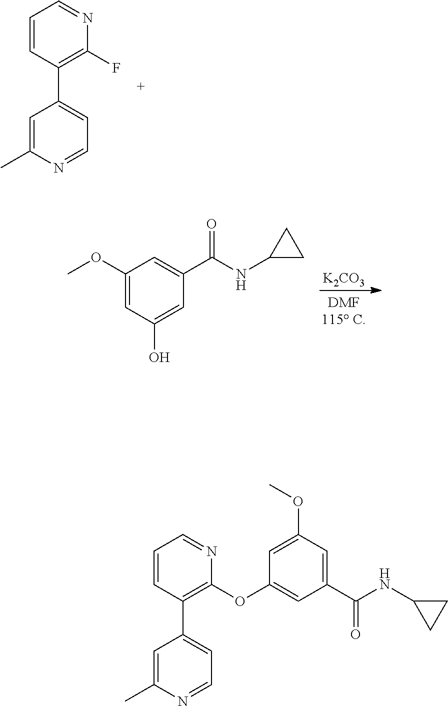

N-cyclopropyl-3-methoxy-5-((2′-methyl-[3,4′-bipyridin]-2-yl)oxy)benzamide (Compound 98): 2-fluoro-2′-methyl-3,4′-bipyridine (627 mg, 3.33 mmol, 1.0 eq), N-cyclopropyl-3-hydroxy-5-methoxybenzamide (690 mg, 3.33 mmol, 1 eq) and K₂CO₃ (1.84 g, 13.33 mmol, 4 eq) were added to a round-bottom flask with a magnetic bar, then 20 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 200 ml ethyl acetate and washed with brine. The organic phase was dried over anhydrous Na₂SO₄, and concentrated in vacuo. The residue was purified by silica gel flash chromatography to afford the product. (1.2 g, yield=96.1%, purity=95.75%)

TLC R_{f}=0.35 (DCM/MeOH=20/1)

MS (ESI⁺): m/z=376.20 (M+1)

¹H NMR: (400 MHz, DMSO-d6) δ 8.54 (d, J=5.2 Hz, 1H), 8.41 (d, J=4.2 Hz, 1H), 8.20 (dd, J=4.9, 1.9 Hz, 1H), 8.04 (dd, J=7.5, 1.9 Hz, 1H), 7.61-7.57 (m, 1H), 7.54 (dd, J=5.3, 1.7 Hz, 1H), 7.30 (dd, J=7.5, 4.9 Hz, 1H), 7.25 (dd, J=2.4, 1.4 Hz, 1H), 7.18 (t, J=1.7 Hz, 1H), 6.94 (t, J=2.2 Hz, 1H), 3.80 (s, 3H), 2.82 (tq, J=7.8, 4.0 Hz, 1H), 2.54 (s, 3H), 0.68 (td, J=7.1, 4.6 Hz, 2H), 0.59-0.49 (m, 2H).

### Example 99

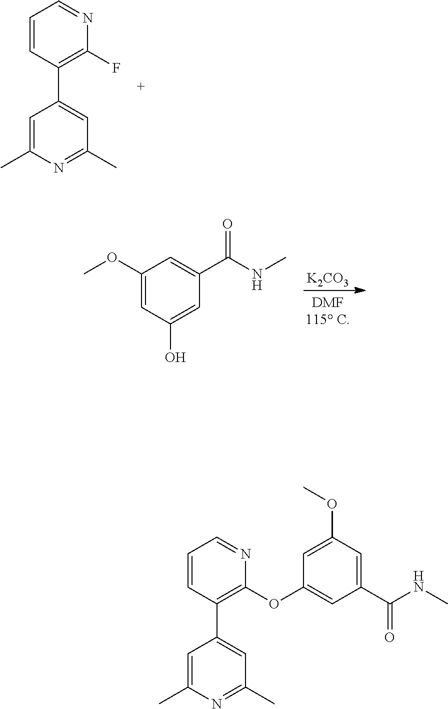

3-((2′,6′-dimethyl-[3,4′-bipyridin]-2-yl)oxy)-5-methoxy-N-methylbenzamide (Compound 99): 2-fluoro-2′,6′-dimethyl-3,4′-bipyridine (121 mg, 0.6 mmol, 1.2 eq), 3-hydroxy-5-methoxy-N-methylbenzamide (91 mg, 0.5 mmol, 1 eq) and K₂CO₃ (276 mg, 2.0 mmol, 4 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product. (106 mg, yield=58.4%, purity=82.35%)

TLC R_{f}=0.25 (DCM/MeOH=10/1)

MS (ESI⁺): m/z=364.10 (M+1)

### Example 100

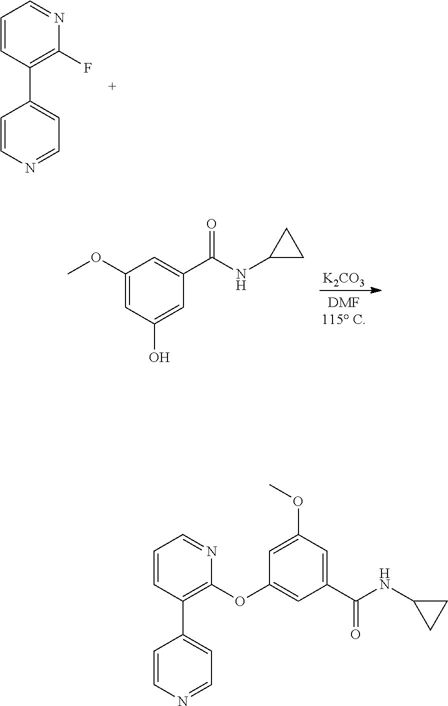

3-([3,4′-bipyridin]-2-yloxy)-N-cyclopropyl-5-methoxybenzamide (Compound 100): 2-fluoro-3,4′-bipyridine (87 mg, 0.5 mmol, 1.0 eq), N-cyclopropyl-3-hydroxy-5-methoxybenzamide (104 mg, 0.5 mmol, 1.0 eq) and K₂CO₃ (138 mg, 1.0 mmol, 2.0 eq) were added to a round-bottom flask with a magnetic bar, then 20 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product. (160 mg, yield=88.64f %, purity=87.37%)

TLC R_{f}=0.65 (DCM/MeOH=20/1)

MS (ESI⁺): m/z=362.20 (M+1)

¹H NMR: (400 MHz, DMSO-d6) δ 8.77-8.61 (m, 2H), 8.41 (d, J=4.2 Hz, 1H), 8.21 (dd, J=4.8, 1.9 Hz, 1H), 8.07 (dd, J=7.5, 1.9 Hz, 1H), 7.80-7.69 (m, 2H), 7.32 (dd, J=7.5, 4.9 Hz, 1H), 7.26 (dd, J=2.4, 1.4 Hz, 1H), 7.18 (t, J=1.7 Hz, 1H), 6.96 (t, J=2.2 Hz, 1H), 3.80 (s, 3H), 2.82 (tq, J=7.8, 4.0 Hz, 1H), 0.67 (dt, J=6.9, 3.2 Hz, 2H), 0.55 (dt, J=7.1, 4.4 Hz, 2H).

### Example 101

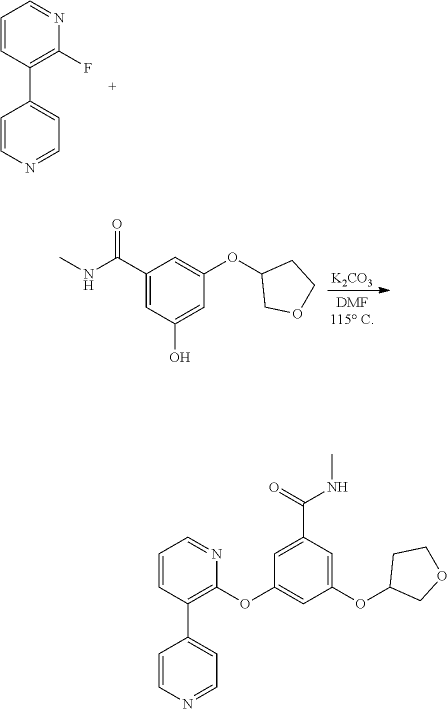

3-([3,4′-bipyridin]-2-yloxy)-N-methyl-5-((tetrahydrofuran-3-yl)oxy)benzamide (Compound 101): 2-fluoro-3,4′-bipyridine (104 mg, 0.6 mmol, 1.2 eq), 3-hydroxy-N-methyl-5-((tetrahydrofuran-3-yl)oxy)benzamide (118.6 mg, 0.5 mmol, 1.0 eq) and K₂CO₃ (276 mg, 2.0 mmol, 4 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product. (157 mg, yield=80.3%, purity=99.58%)

TLC R_{f}=0.3 (DCM/MeOH=40/1)

MS (ESI⁺): m/z=392.40 (M+1)

### Example 102. Cell Culture and Treatment

Six lung cancer cell lines (NCI-H23, NCI-H460, NCI-H596, NCI-H2170, Calu-6 and A549), two colon cancer cell lines (HCT116 and SW460), one breast cancer cell line MDA-MB-231, one gastric cancer cell line NCI-N87, one prostate cancer cell line DU145, one cervical cancer cell line Hela, and one glioblastoma cell line T98G were obtained from ATCC. The T2 HCC cell line was derived from a mouse liver cancer model initiated by a transgene of MYC (Reference). All cell lines were cultured in DMEM (Gibco, Cleveland, TN, USA) supplemented with 5% fetal bovine serum (Gibco), penicillin (100 U/mL)-streptomycin (100 μg/mL) (Gibco, Cat. No. 15140-122), 2 mM L-glutamine (Gibco, 200 mM solution, Cat. No. 25030081), and 1 mM sodium pyruvate (Gibco, 100 mM solution, Cat. No. 11360070) at 37° C. in a humidified incubator that was maintained at 5% CO2.

### Example 103. Phenotypic Screening Assays

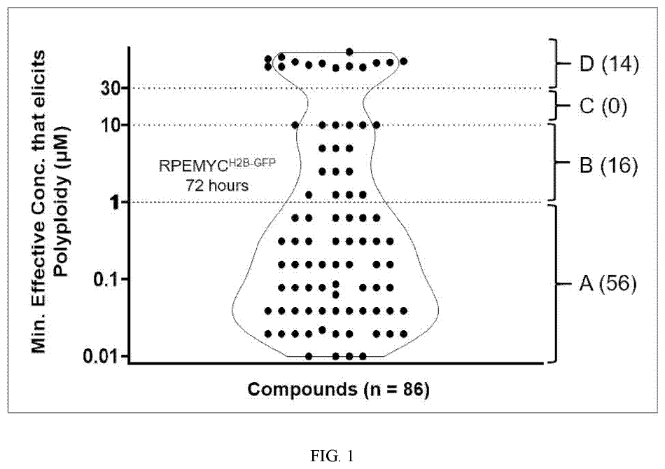

We have Previously Developed a Mechanism-Informed Phenotypic Screening Assay to identify novel antimitotic agents (Li et al. 2019). Equipped with a prior understanding of the versatile functions of the chromosomal passenger protein (CPP) complex in orchestrating karyokinesis and cytokinesis, the screening assay is to score for phenotypes typically seen when the CPP complex is disabled. Specifically, the parameters for a positive hit are a temporary elevation of mitotic index (MI) at 24 hours of drug treatment and an accumulation of polyploid cells at 48 hours of drug treatment, indicative of mitotic arrest and cytokinetic failure respectively. These parameters exclude compounds that elicit only a prolonged arrest of cells in mitosis, a phenotype typically provoked by spindle toxins. The screening procedure is briefly summarized here. RPEMYC^{H2B-GFP} cells engineered to express a Histone 2B-EGFP fusion protein were passaged as batches of 96-well plates, 18-24 hours before exposure to the chemical compounds of the present disclosure at concentrations from 20 nM to 20 μM. At 24, 48 or 72 hours after initiation of treatment, cells were analyzed for either an arrest in mitosis or a change in DNA content by GE IN-Cell Analyzer 2000. Testing results of 85 compounds were summarized in FIG. 1 and Table 2. As set forth in table 2 below, a value of greater than or equal to 1 μM and less than or equal to 1.0 μM is marked e “A”; a value greater than 1.00 nM and less than or equal to 10.0 μM is marked “B”; a value greater than 10.0 μM and less than or equal to 30.0 μM is marked “C”; and a value greater than 30.0 μM is marked “D.”

TABLE 2

| | | Min. Effective | Along | |
|---|---|---|---|---|
| | Mitotic | conc. for | with | Proliferative |
| | Arrest | polyploidy | Cell | arrest |
| NO. | (≥5%) | (≥5%) | Death | (>50%) |
| 1 | A | A | A | A |
| 2 | A | A | A | A |
| 3 | D | D | D | D |
| 4 | A | A | A | A |
| 5 | B | B | B | B |
| 6 | A | A | A | A |
| 7 | A | A | A | A |
| 8 | A | A | A | A |
| 9 | A | A | A | A |
| 10 | A | A | A | A |
| 11 | A | A | A | A |
| 12 | A | A | A | A |
| 13 | A | A | A | A |
| 14 | A | A | A | A |
| 15 | A | A | A | A |
| 16 | B | B | B | B |
| 17 | A | A | A | A |
| 18 | A | A | A | A |
| 19 | A | A | A | A |
| 20 | A | A | A | A |
| 21 | A | A | A | A |
| 22 | D | D | D | D |
| 23 | A | A | A | A |
| 24 | D | D | D | D |
| 25 | A | A | A | A |
| 26 | A | A | A | A |
| 27 | A | A | A | A |
| 28 | A | A | A | A |
| 29 | A | A | A | A |
| 30 | A | A | A | A |
| 31 | A | A | A | A |
| 32 | A | A | A | A |
| 33 | A | A | A | A |
| 34 | A | A | A | A |
| 35 | A | A | A | A |
| 36 | A | A | A | A |
| 37 | A | A | A | A |
| 38 | A | A | A | A |
| 39 | A | A | A | A |
| 40 | A | A | A | A |
| 41 | A | A | A | A |
| 42 | A | A | A | A |
| 43 | A | A | A | A |
| 44 | A | A | A | A |
| 45 | A | A | A | A |
| 46 | A | A | A | A |
| 47 | A | A | A | A |
| 48 | B | B | B | B |
| 49 | A | A | A | A |
| 50 | A | A | A | A |
| 51 | D | D | D | D |
| 52 | A | A | A | A |
| 53 | D | D | D | D |
| 54 | D | D | D | D |
| 55 | B | B | B | B |
| 56 | A | A | A | A |
| 57 | B | B | B | B |
| 58 | A | A | A | A |
| 59 | B | B | B | B |
| 60 | A | A | A | A |
| 61 | A | A | A | A |
| 62 | B | B | B | B |
| 63 | A | A | A | A |
| 64 | B | B | B | B |
| 65 | B | B | B | B |
| 66 | A | A | A | A |
| 67 | B | B | B | B |
| 68 | A | A | A | A |
| 69 | A | A | A | A |
| 70 | A | A | A | A |
| 71 | D | D | D | D |
| 72 | B | B | B | B |
| 73 | D | D | D | D |
| 74 | B | B | B | B |
| 75 | B | B | B | B |
| 76 | D | D | D | D |
| 77 | D | D | D | D |
| 78 | D | D | D | D |
| 79 | D | D | D | D |
| 80 | B | B | B | B |
| 81 | B | B | B | B |
| 82 | B | B | B | B |
| 83 | D | D | D | D |
| 84 | C | D | D | D |
| 85 | A | A | A | A |
| 86 | A | A | A | A |
| 87 | A | A | A | A |
| 88 | A | A | A | A |
| 89 | A | A | A | A |
| 90 | A | A | A | A |
| 91 | A | A | A | A |
| 92 | A | A | A | A |
| 93 | inactive | inactive | inactive | inactive |
| 94 | A | A | A | A |
| 95 | A | A | A | A |
| 96 | A | A | A | A |
| 97 | A | A | A | A |
| 98 | A | A | A | A |
| 99 | A | A | A | A |
| 100 | A | A | A | A |
| 101 | A | A | A | A |

### Example 104. Soft Agar Colony Formation Assay

Measuring the ability of cells to grow in soft agar has been popularly believed as the gold standard assay for cellular transformation in vitro. In the Soft Agar Assay, cells grow from single cells to cell colonies in a semi-solid agar solution that keeps them away from the solid surface and allows growth in an anchorage-independent way.

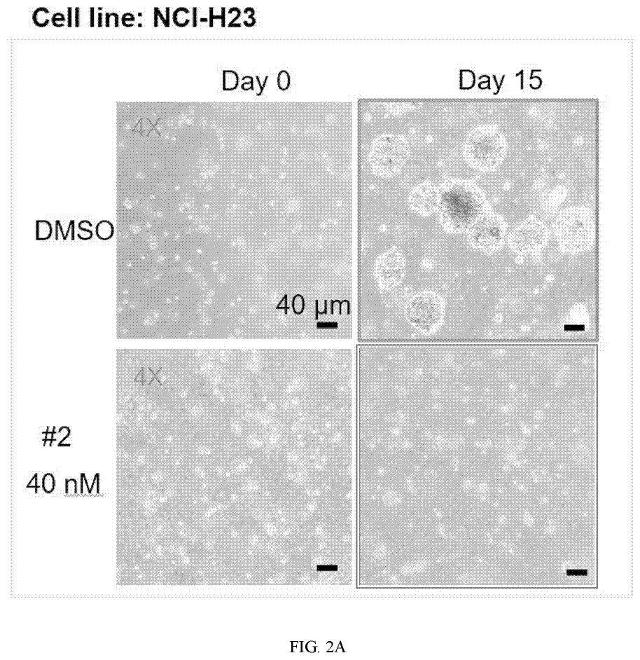

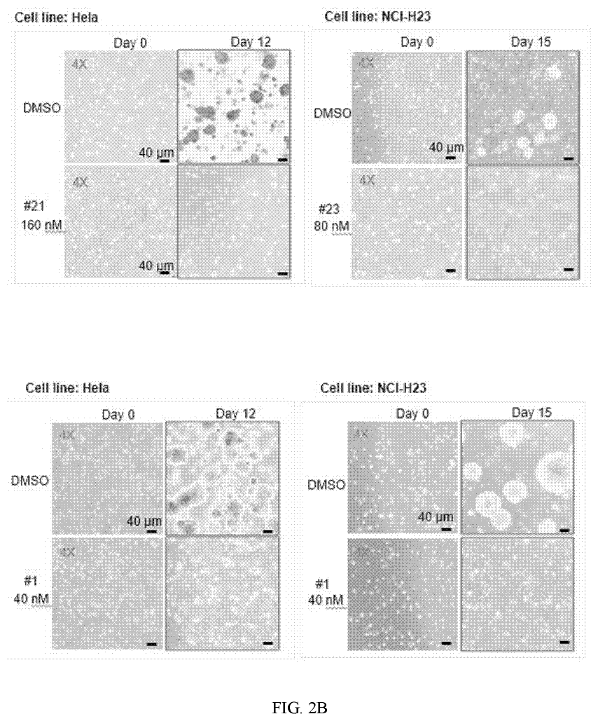

The anchorage-independent growth of cells is one of the hallmarks of cancer cells. Normal epithelial cells are supported by basement membranes that provide survival and proliferative signals while undergo a type of apoptosis called anoikis when lose their attachment to the extracellular matrix. Cancer cells, in contrast, evade attachment-induced apoptosis, leading to uncontrolled proliferation and metastasis. The Soft Agar Colony Formation Assay allows testing of the therapeutic efficacy of compounds against anchorage-independent 3D growth of cancer cells in vitro. The assay was performed in 6-well plates with two layers of agar. For the first, 0.75% agar in DMEM medium was melted in a microwave oven and poured to form a bottom layer. Once solidified, 10-100K cells in 1 ml of DMEM containing 0.35% agar was added to form the top layer, which was later covered with 0.5 ml of DMEM. Cell culture medium was changed once every two days until colonies were ready to photograph. We test the antitumor activity of select compounds in soft agar colony formation assays. Data are summarized in FIG. 2. Both compound #1 and compound #21 effectively suppressed the anchorage-independent growth of a cervical cancer cell line Hela. Similarly, all compound tested, including #1, #2, and #23 completely blocked the growth of a human lung cancer cell line NCI-H23 in soft agar. The suppression of growth of these two cancer cell lines in 3D culture is consistent with the potent impact of these compounds on cellular proliferation in 2D culture.

### Example 105. The MTT Assay of Cellular Proliferation and Determination of EC₅₀

The MTT assay measures cellular metabolic activity as a proxy for cell viability and involves the conversion of the water-soluble yellow dye MTT [3-(4,5-dimethylthiazol-2-yl)-2,5-diphenyltetrazolium bromide] into an insoluble purple formazan by the action of mitochondrial reductase. Formazan is then solubilized and its concentration is determined by measuring the optical density (OD) value at a wavelength of 570 nm. The value is in proportional to the number of live cells with excellent linearity up to ˜10⁶ cells per well. The MTT assay was used to determine the EC₅₀ value, the concentration of a compound that leads to 50% inhibition of cellular proliferation. Briefly, cells were split when growing to the mid-Log phase. Cells in 100 μL of culture medium were seeded into each well of 96-well microplates and cultivated for 15˜24 hours to reach a confluence of 20˜30% and were then exposed to drugs at concentrations ranging from 1 nM to 10 μM. At the endpoint, 20 μL of a MTT stock solution in DMSO (5 mg/mL) was added to each well that contains 1001 μL of DMEM. The microplates were left in the cell culture incubator for 3˜4 h before subjected to solubilization and determination of formazan at A570 in a microplate reader (BioTek ELX808iu). The results of these assays are summarized in Table 3. As set forth in table 2 below, a value of greater than or equal to 1 nM and less than or equal to 1.0 μM is marked “A”; a value greater than 1.00 μM and less than or equal to 10.0 μM is marked “B”; a value greater than 10.0 μM and less than or equal to 30.0 μM is marked “C”; and a value greater than 30.0 μM is marked “D.” The series of compounds displayed potent activity in all human cancer cell lines tested, including six lung cancer cell lines (NCI-H23, NCI-H460, NCI-H596, NCI-H2170, Calu-6 and A549), two colon cancer cell lines (HCT116 and SW460), one breast cancer cell line MDA-MB-231, one gastric cancer cell line NCI-N87, one prostate cancer cell line DU145, one cervical cancer cell line Hela, one glioblastoma cell line T98 G, and one liver cancer cell line T2 HCC. Therefore, these compounds might hold a broad utility in the treatment of a large variety of human malignancies.

TABLE 3
MECP: Minimal effective concentration that elicits polyploidy
EC50: Concentration that inhibits 50% of proliferation
A: 1 nM < conc. ≤ 1 μM
B: 1 μM < conc. ≤ 10 μM
C: 10 μM < conc. ≤ 30 μM
D: 30 μM < conc.
“—” Indicates no assays in this cell line

| | | | | | | | | | | MDA- | | | | | |
|---|---|---|---|---|---|---|---|---|---|---|---|---|---|---|---|
| | | NCI- | NCI- | NCI- | NCI- | | | | | MB- | | NCI- | | Calu- | T2 |
| Compound | | H23 | H460 | H596 | H2170 | A549 | HCT116 | SW480 | T98G | 231 | Hela | N87 | DU145 | 6 | HCC |
| #19 | MECP | A | A | A | A | B | A | — | A | A | A | A | B | A | A |
| | EC50 | A | B | A | A | B | A | — | A | A | A | A | B | A | A |
| #21 | MECP | A | B | A | A | — | A | — | B | A | A | A | A | A | A |
| | EC50 | A | C | B | A | — | A | — | B | A | A | A | A | A | A |
| #1 | MECP | A | B | A | A | B | A | — | A | A | A | A | A | A | A |
| | EC50 | A | C | C | A | C | A | — | A | A | A | A | A | A | A |
| #23 | MECP | A | B | A | A | B | A | A | A | A | A | A | A | A | A |
| | EC50 | A | C | A | A | C | A | A | C | A | A | A | A | A | B |
| #2 | MECP | A | B | A | A | B | A | A | A | A | A | A | A | A | A |
| | EC50 | A | C | A | A | C | A | A | A | A | A | A | A | A | A |
| #4 | MECP | A | — | — | — | A | A | A | — | A | A | A | — | — | A |
| | EC50 | A | — | — | — | A | A | A | — | A | A | B | — | — | A |
| #3 | MECP | A | — | — | — | A | A | A | — | A | A | A | — | — | A |
| | EC50 | A | — | — | — | A | A | A | — | A | A | A | — | — | A |
| #28 | MECP | A | — | — | — | A | A | A | — | A | A | A | — | — | A |
| | EC50 | A | — | — | — | A | A | A | — | A | A | A | — | — | A |
| #7 | MECP | A | — | — | — | A | A | A | — | A | A | A | — | — | A |
| | EC50 | A | — | — | — | A | A | A | — | A | A | A | — | — | A |
| #29 | MECP | A | — | — | — | A | A | A | — | A | A | A | — | — | A |
| | EC50 | A | — | — | — | A | A | A | — | A | A | A | — | — | A |
| #8 | MECP | A | — | — | — | A | A | A | — | A | A | A | — | — | A |
| | EC50 | A | — | — | — | A | A | A | — | A | A | A | — | — | A |
| #36 | MECP | A | — | — | — | A | A | A | — | A | A | A | — | — | A |
| | EC50 | A | — | — | — | A | A | A | — | A | A | A | — | — | A |
| #37 | MECP | A | — | — | — | A | A | A | — | A | A | A | — | — | A |
| | EC50 | A | — | — | — | A | A | A | — | A | A | A | — | — | A |
| #38 | MECP | A | — | — | — | A | A | A | — | A | A | A | — | — | A |
| | EC50 | A | — | — | — | A | A | A | — | A | A | A | — | — | A |
| #39 | MECP | A | — | — | — | A | A | A | — | A | A | A | — | — | A |
| | EC50 | A | — | — | — | A | A | A | — | A | A | A | — | — | A |
| #9 | MECP | A | — | — | — | A | A | A | — | A | A | A | — | — | A |
| | EC50 | A | — | — | — | A | A | A | — | A | A | A | — | — | A |
| #40 | MECP | A | — | — | — | A | A | A | — | A | A | A | — | — | A |
| | EC50 | A | — | — | — | A | A | A | — | A | A | A | — | — | A |
| #30 | MECP | A | — | — | — | A | A | A | — | A | A | A | — | — | A |
| | EC50 | A | — | — | — | A | A | A | — | A | A | A | — | — | A |
| #10 | MECP | A | — | — | — | A | A | A | — | A | A | A | — | — | A |
| | EC50 | A | — | — | — | A | A | A | — | A | A | A | — | — | A |
| #41 | MECP | A | — | — | — | A | A | A | — | A | A | A | — | — | A |
| | EC50 | A | — | — | — | A | A | A | — | A | A | A | — | — | A |
| #31 | MECP | A | — | — | — | A | A | A | — | A | A | A | — | — | A |
| | EC50 | A | — | — | — | A | A | A | — | A | A | A | — | — | A |
| #26 | MECP | A | — | — | — | A | A | A | — | A | A | A | — | — | A |
| | EC50 | A | — | — | — | A | A | A | — | A | A | A | — | — | A |
| #13 | MECP | A | — | — | — | A | A | A | — | A | A | A | — | — | A |
| | EC50 | A | — | — | — | A | A | A | — | A | A | A | — | — | A |
| #43 | MECP | A | — | — | — | A | A | A | — | A | A | A | — | — | A |
| | EC50 | A | — | — | — | A | A | A | — | A | A | A | — | — | A |
| #46 | MECP | A | — | — | — | A | A | A | — | A | A | A | — | — | A |
| | EC50 | A | — | — | — | A | A | A | — | A | A | A | — | — | A |
| #34 | MECP | A | — | — | — | A | A | A | — | A | A | A | — | — | A |
| | EC50 | A | — | — | — | A | A | A | — | A | A | A | — | — | A |
| #32 | MECP | A | — | — | — | A | A | A | — | A | A | A | — | — | A |
| | EC50 | A | — | — | — | A | A | A | — | A | A | A | — | — | A |
| #17 | MECP | A | — | — | — | A | A | — | — | A | A | A | — | — | A |
| | EC50 | A | — | — | — | C | A | — | — | A | A | A | — | — | A |
| #52 | MECP | A | — | — | — | A | A | A | — | A | A | A | — | — | A |
| | EC50 | A | — | — | — | B | A | A | — | A | A | A | — | — | A |
| #14 | MECP | A | — | — | — | A | A | A | — | A | A | A | — | — | A |
| | EC50 | A | — | — | — | A | A | A | — | A | A | A | — | — | A |
| #27 | MECP | A | — | — | — | A | A | A | — | A | A | A | — | — | A |
| | EC50 | A | — | — | — | A | A | A | — | A | A | A | — | — | A |
| #42 | MECP | A | — | — | — | A | A | A | — | A | A | A | — | — | A |
| | EC50 | A | — | — | — | A | A | A | — | A | A | A | — | — | A |
| #35 | MECP | A | — | — | — | A | A | A | — | A | A | A | — | — | A |
| | EC50 | A | — | — | — | A | A | A | — | A | A | A | — | — | A |
| #63 | MECP | A | — | — | — | A | A | A | — | A | A | A | — | — | A |
| | EC50 | A | — | — | — | A | A | A | — | A | A | A | — | — | A |
| #45 | MECP | A | — | — | — | A | A | A | — | A | A | A | — | — | A |
| | EC50 | A | — | — | — | A | A | A | — | A | A | A | — | — | A |
| #12 | MECP | A | — | — | — | A | A | A | — | A | A | A | — | — | A |
| | EC50 | A | — | — | — | A | A | A | — | A | A | A | — | — | A |

### Example 106. Xenograft Assays

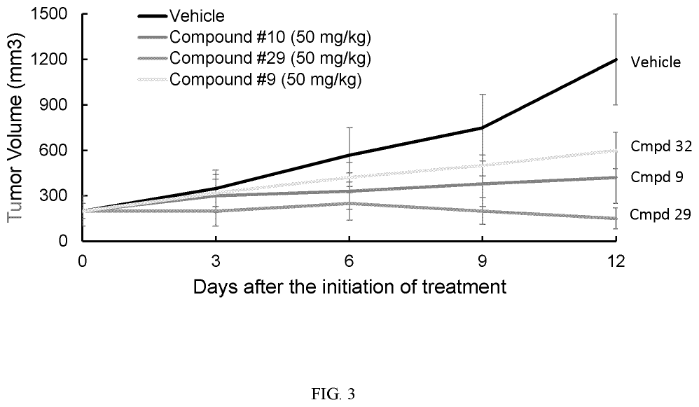

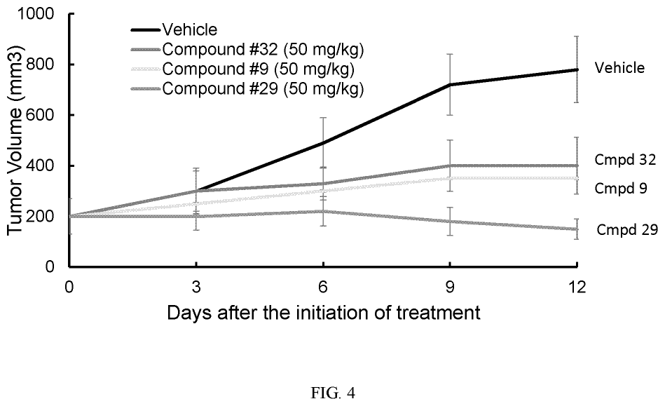

Xenografts were initiated in immunocompromised (Nu/Nu) mice with the human lung adenocarcinoma cell line NCI-H23 (FIG. 3) and the human colon cancer cell line HCT116 (FIG. 4). Five million cells were injected subcutaneously into each mouse and treatment was initiated when the average tumor volumes reached 150 mm3 (n=5/groups). Tumor-bearing mice were randomized into different groups to receive either vehicle or indicated compounds. The compounds were administered through oral gavage twice a day for 9 days. Day 0 on the x-axis indicates the day that treatment was initiated. For these experiments, all compounds were first dissolved in DMSO and then diluted 1:10 into a mixture containing 50% PEG300 and 49% PBS, 1% Tween 80, pH2.2. 100 ul of drug solution was administered with each dose. Tumor volumes were determined once every three days and are calculated from digital caliper raw data by using the formula: Volume (mm3)=(L×W²)/2. The value W (Width) is the smaller of two perpendicular tumor axes and the value L (Length) is the larger of two perpendicular axes. Mean tumor volume growth curves and means are calculated for each treatment group. Compounds #9, #10, #29 and #32 demonstrated the therapeutic efficacy in these mouse tumor models, suppressing the tumor growth and even eliciting tumor regression by compound #9 (FIG. 3 and FIG. 4).

### REFERENCES

1. Victor J. Cee, Laurie B. Schenkel, Brian L. Hodous et al., J. Med. Chem. 2010, 53, 17, 6368-6377.
2. Shanghua Xia, Lu Gan, Kailiang Wang, Zheng Li, Dawei Ma. J. Am. Chem. Soc. 2016, 138, 13493-13496.

1. A compound of formula I: 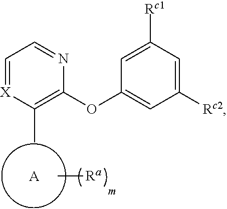
or a pharmaceutically acceptable salt,
wherein
X is —CH═ or —N═;
Group A is optionally substituted phenyl or optionally substituted 6-membered heteroaryl;
each R^{a} is independently selected from the group consisting of halogen, —CN, —OH, —OR¹, —SR¹, —NH₂, —NR²R³, —C(O)R¹, —C(O)OH, —C(O)OR¹, —C(O)NH₂, —C(O)NR²R³, optionally substituted C₁-C₇ aliphatic, optionally substituted phenyl, optionally substituted 5-10 membered heteroaryl, and optionally substituted 5-10 membered heterocyclyl;
R^{c1} is selected from the group consisting of C₁-C₆ haloalkyl, halogen, —OR¹, —C(O)OR¹, and optionally substituted 5-6 membered heteroaryl;
R^{c2} is selected from the group consisting of halogen, —CN, —OH, —OR¹, —NH₂, —NR²R³ optionally substituted C₁-C₇ aliphatic, optionally substituted phenyl, —C(O)R¹, —C(O)OH, —C(O)OR¹, —C(O)NH₂, —C(O)NR²R³, optionally substituted 5-10 membered heteroaryl, and optionally substituted 5-10 membered heterocyclyl;
each R¹ is independently selected from the group consisting of optionally substituted C₁-C₆ aliphatic, optionally substituted phenyl, optionally substituted 5-10 membered heteroaryl, and optionally substituted 5-10 membered heterocyclyl;
each R² is independently selected from the group consisting of —C(O)R¹, —C(O)OR¹, —C(O)NR¹R³, optionally substituted C₁-C₆ aliphatic, optionally substituted phenyl, optionally substituted 5-10 membered heteroaryl, and optionally substituted 5-10 membered heterocyclyl;
each R³ is independently selected from the group consisting of hydrogen, —C(O)R′, —C(O)OR¹, —C(O)NHR¹, optionally substituted C₁-C₆ aliphatic, optionally substituted phenyl, optionally substituted 5-10 membered heteroaryl, and optionally substituted 5-10 membered heterocyclyl; and
m is 0, 1, 2, 3, 4, or 5.

2. The compound of claim 1, wherein the compound is a compound of formula (I-a): 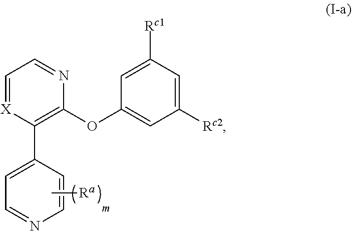
or a pharmaceutically acceptable salt thereof.

3. The compound of claim 1, wherein the compound is a compound of formula (I-b): 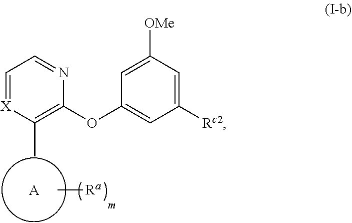
or a pharmaceutically acceptable salt thereof.

4. The compound of claim 1, wherein the compound is a compound of formula (I-c): 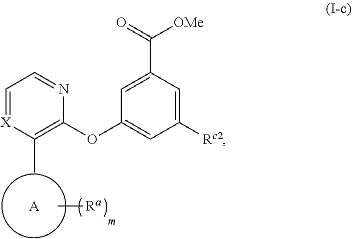
or a pharmaceutically acceptable salt thereof.

5. The compound of claim 1, wherein the compound is a compound of formula (I-d): 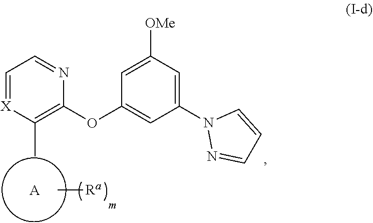
or a pharmaceutically acceptable salt thereof.

6. The compound of any of claims 1-5, wherein R^{∘}1 is —OMe.

7. The compound of any of claims 1-5, wherein R^{∘}1 is —OEt.

8. The compound of any of claims 1-5, wherein R^{∘}1 is CF₃.

9. The compound of any of claims 1-5, wherein R^{∘}1 is —OR¹ and R¹ is substituted C₁-C₆ aliphatic.

10. The compound of any of claims 1-9, wherein R^{c2} is —C(O)R^{1′}—C(O)OR¹, or —C(O)NH₂.

11. The compound of any of claims 1-9, wherein R² is a substituted 5-10 membered heteroaryl.

12. The compound of any of claims 1-11, wherein Group A is optionally substituted 6-membered heteroaryl.

13. The compound of claim 12 wherein Group A is 6 optionally substituted pyridine or optionally substituted pyrimidine.

14. The compound of any of claims 1-11, wherein Group A is optionally substituted phenyl.

15. A compound selected from the group consisting of

| NO. | Structure |
|---|---|
| 1 | 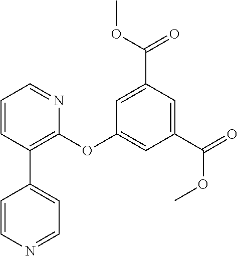 [SMILES: COC(=O)c1cc(Oc2ncccc2-c2ccncc2)cc(C(=O)OC)c1] |
| 2 | 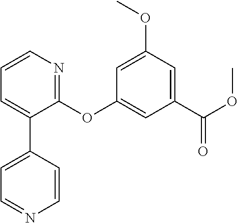 [SMILES: COC(=O)c1cc(OC)cc(Oc2ncccc2-c2ccncc2)c1] |
| 3 | 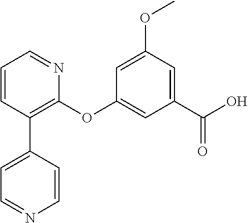 [SMILES: COc1cc(Oc2ncccc2-c2ccncc2)cc(C(=O)O)c1] |
| 4 | 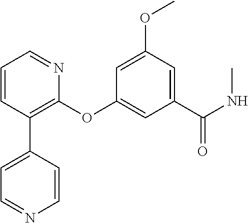 [SMILES: CNC(=O)c1cc(OC)cc(Oc2ncccc2-c2ccncc2)c1] |
| 5 | 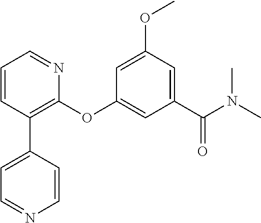 [SMILES: COc1cc(Oc2ncccc2-c2ccncc2)cc(C(=O)N(C)C)c1] |
| 6 | 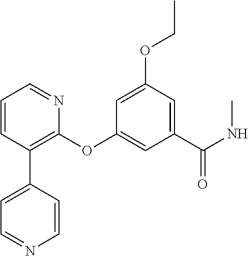 [SMILES: CCOc1cc(Oc2ncccc2-c2ccncc2)cc(C(=O)NC)c1] |
| 7 | 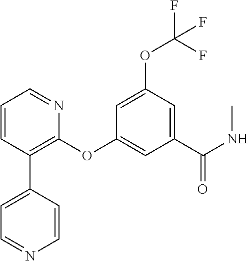 [SMILES: CNC(=O)c1cc(Oc2ncccc2-c2ccncc2)cc(OC(F)(F)F)c1] |
| 8 | 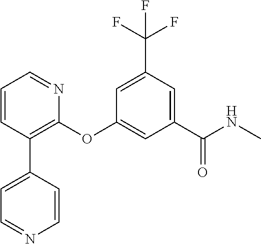 [SMILES: CNC(=O)c1cc(Oc2ncccc2-c2ccncc2)cc(C(F)(F)F)c1] |
| 9 | 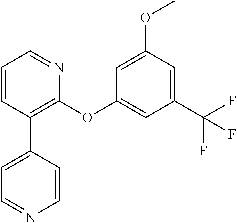 [SMILES: COc1cc(Oc2ncccc2-c2ccncc2)cc(C(F)(F)F)c1] |
| 10 | 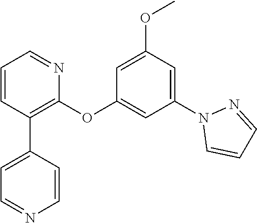 [SMILES: COc1cc(Oc2ncccc2-c2ccncc2)cc(-n2cccn2)c1] |
| 11 | 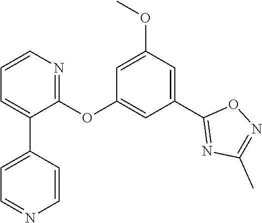 [SMILES: COc1cc(Oc2ncccc2-c2ccncc2)cc(-c2nc(C)no2)c1] |
| 12 | 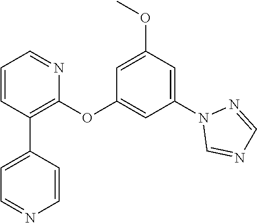 [SMILES: COc1cc(Oc2ncccc2-c2ccncc2)cc(-n2cncn2)c1] |
| 13 | 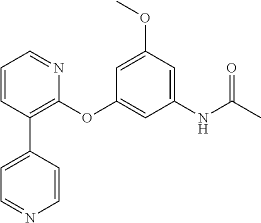 [SMILES: COc1cc(NC(C)=O)cc(Oc2ncccc2-c2ccncc2)c1] |
| 14 | 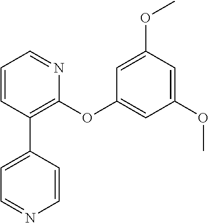 [SMILES: COc1cc(OC)cc(Oc2ncccc2-c2ccncc2)c1] |
| 15 | 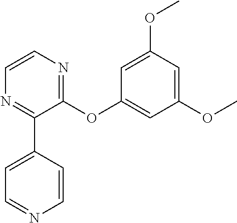 [SMILES: COc1cc(OC)cc(Oc2nccnc2-c2ccncc2)c1] |
| 17 | 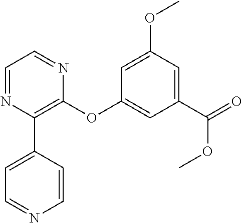 [SMILES: COC(=O)c1cc(OC)cc(Oc2nccnc2-c2ccncc2)c1] |
| 18 | 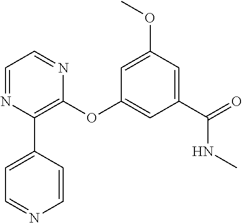 [SMILES: CNC(=O)c1cc(OC)cc(Oc2nccnc2-c2ccncc2)c1] |
| 19 | 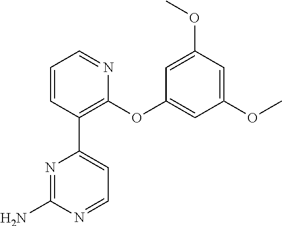 [SMILES: COc1cc(OC)cc(Oc2ncccc2-c2ccnc(N)n2)c1] |
| 21 | 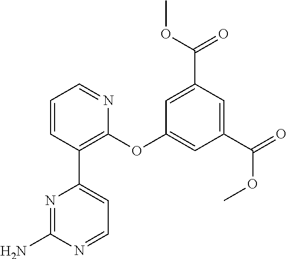 [SMILES: COC(=O)c1cc(Oc2ncccc2-c2ccnc(N)n2)cc(C(=O)OC)c1] |
| 22 | 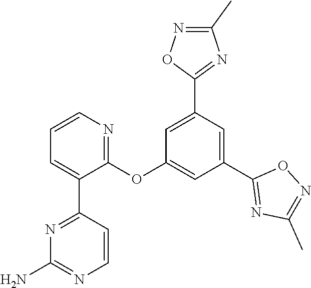 [SMILES: Cc1noc(-c2cc(Oc3ncccc3-c3ccnc(N)n3)cc(-c3nc(C)no3)c2)n1] |
| 23 | 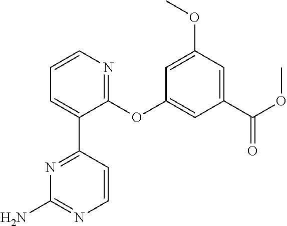 [SMILES: COC(=O)c1cc(OC)cc(Oc2ncccc2-c2ccnc(N)n2)c1] |
| 24 | 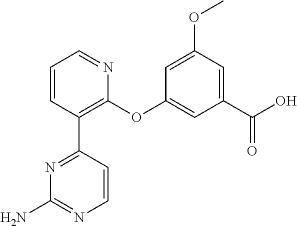 [SMILES: COc1cc(Oc2ncccc2-c2ccnc(N)n2)cc(C(=O)O)c1] |
| 25 | 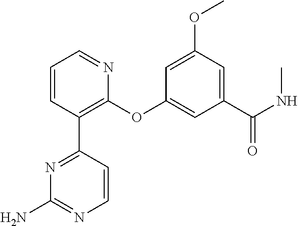 [SMILES: CNC(=O)c1cc(OC)cc(Oc2ncccc2-c2ccnc(N)n2)c1] |
| 26 | 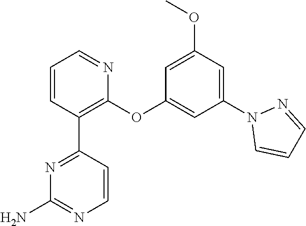 [SMILES: COc1cc(Oc2ncccc2-c2ccnc(N)n2)cc(-n2cccn2)c1] |
| 27 | 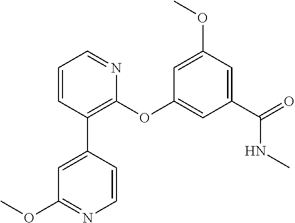 |
| 28 | 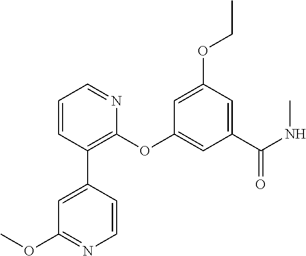 |
| 29 | 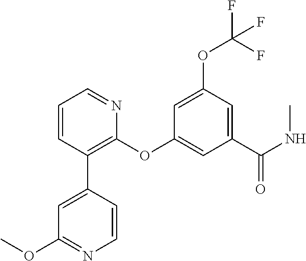 |
| 30 | 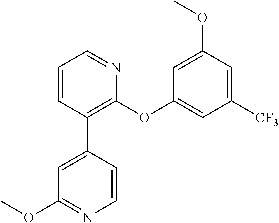 [SMILES: COc1cc(Oc2ncccc2-c2ccnc(OC)c2)cc(C(F)(F)F)c1] |
| 31 | 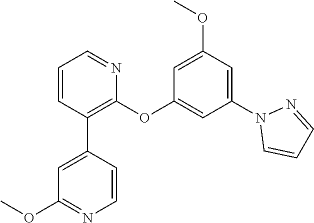 [SMILES: COc1cc(Oc2ncccc2-c2ccnc(OC)c2)cc(-n2cccn2)c1] |
| 32 | 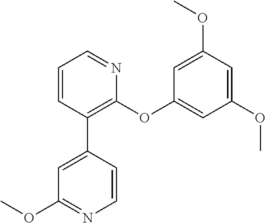 |
| 34 | 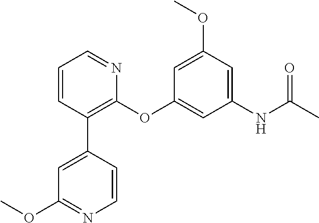 [SMILES: COc1cc(NC(C)=O)cc(Oc2ncccc2-c2ccnc(OC)c2)c1] |
| 35 | 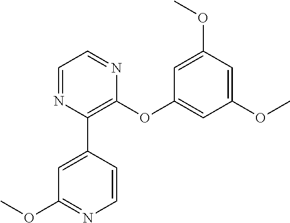 |
| 36 | 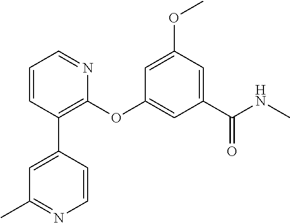 |
| 37 | 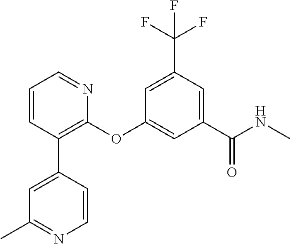 |
| 38 | 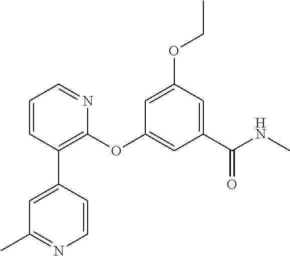 |
| 39 | 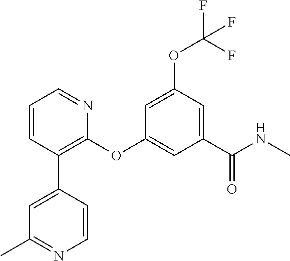 |
| 40 | 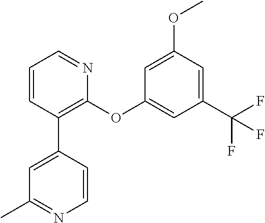 [SMILES: COc1cc(Oc2ncccc2-c2ccnc(C)c2)cc(C(F)(F)F)c1] |
| 41 | 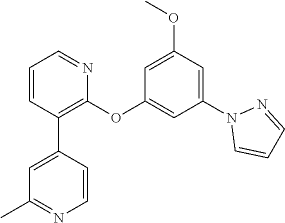 [SMILES: COc1cc(Oc2ncccc2-c2ccnc(C)c2)cc(-n2cccn2)c1] |
| 42 | 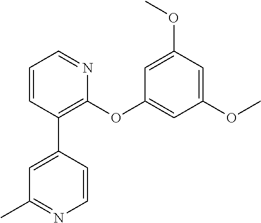 |
| 43 | 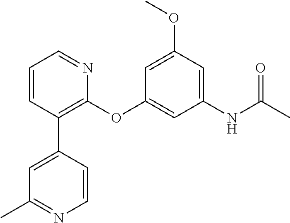 [SMILES: COc1cc(NC(C)=O)cc(Oc2ncccc2-c2ccnc(C)c2)c1] |
| 44 | 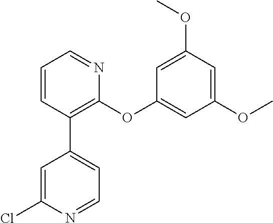 [SMILES: COc1cc(OC)cc(Oc2ncccc2-c2ccnc(Cl)c2)c1] |
| 45 | 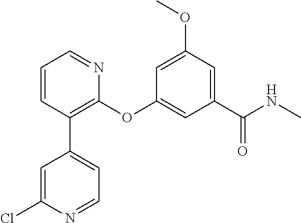 [SMILES: CNC(=O)c1cc(OC)cc(Oc2ncccc2-c2ccnc(Cl)c2)c1] |
| 46 | 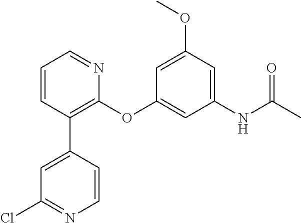 [SMILES: COc1cc(NC(C)=O)cc(Oc2ncccc2-c2ccnc(Cl)c2)c1] |
| 47 | 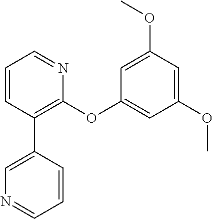 [SMILES: COc1cc(OC)cc(Oc2ncccc2-c2cccnc2)c1] |
| 50 | 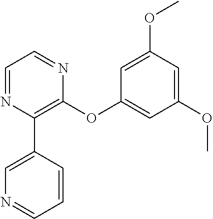 [SMILES: COc1cc(OC)cc(Oc2nccnc2-c2cccnc2)c1] |
| 52 | 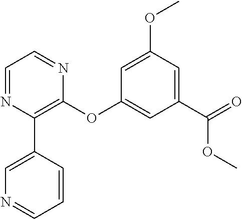 [SMILES: COC(=O)c1cc(OC)cc(Oc2nccnc2-c2cccnc2)c1] |
| 55 | 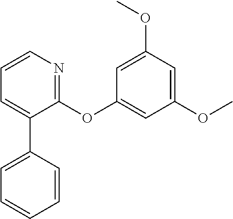 [SMILES: COc1cc(OC)cc(Oc2ncccc2-c2ccccc2)c1] |
| 56 | 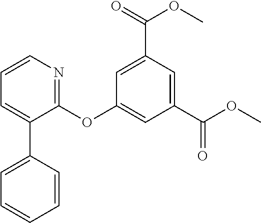 [SMILES: COC(=O)c1cc(Oc2ncccc2-c2ccccc2)cc(C(=O)OC)c1] |
| 57 | 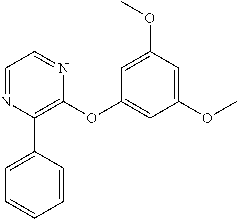 [SMILES: COc1cc(OC)cc(Oc2nccnc2-c2ccccc2)c1] |
| 58 | 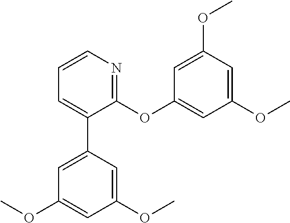 |
| 59 | 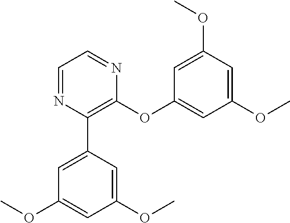 |
| 60 | 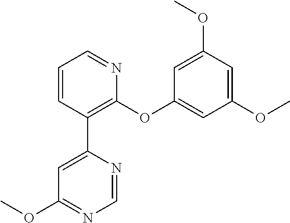 |
| 61 | 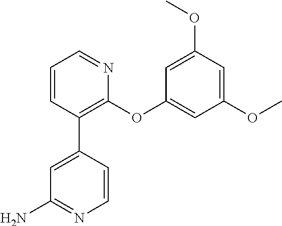 [SMILES: COc1cc(OC)cc(Oc2ncccc2-c2ccnc(N)c2)c1] |
| 62 | 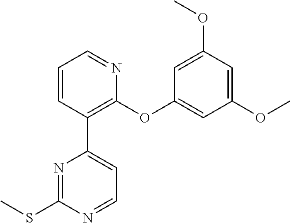 |
| 63 | 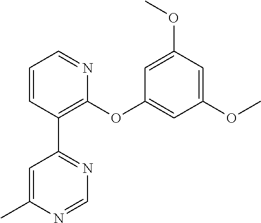 |
| 64 | 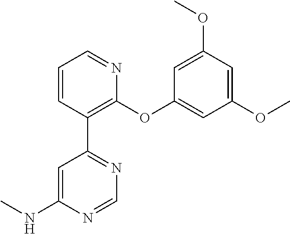 |
| 65 | 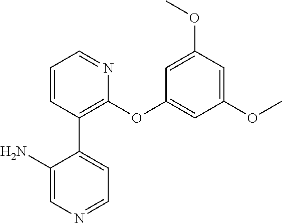 [SMILES: COc1cc(OC)cc(Oc2ncccc2-c2ccncc2N)c1] |
| 66 | 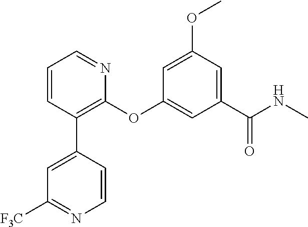 [SMILES: CNC(=O)c1cc(OC)cc(Oc2ncccc2-c2ccnc(C(F)(F)F)c2)c1] |
| 67 | 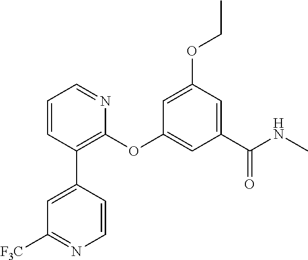 [SMILES: CCOc1cc(Oc2ncccc2-c2ccnc(C(F)(F)F)c2)cc(C(=O)NC)c1] |
| 81 | 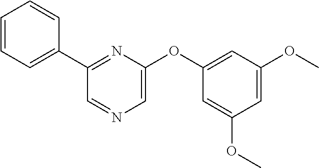 [SMILES: COc1cc(OC)cc(Oc2cncc(-c3ccccc3)n2)c1] |
| 82 | 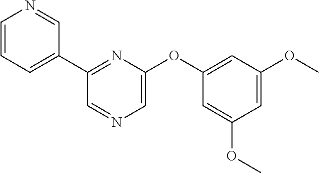 [SMILES: COc1cc(OC)cc(Oc2cncc(-c3cccnc3)n2)c1] |
| 83 | 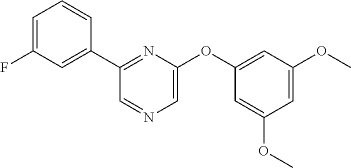 [SMILES: COc1cc(OC)cc(Oc2cncc(-c3cccc(F)c3)n2)c1] |
| 84 | 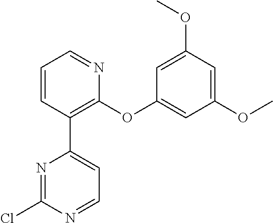 [SMILES: COc1cc(OC)cc(Oc2ncccc2-c2ccnc(Cl)n2)c1] |
| 85 | 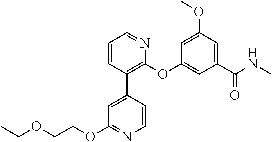 [SMILES: CCOCCOc1cc(-c2cccnc2Oc2cc(OC)cc(C(=O)NC)c2)ccn1] |
| 86 | 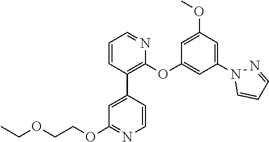 [SMILES: CCOCCOc1cc(-c2cccnc2Oc2cc(OC)cc(-n3cccn3)c2)ccn1] |
| 87 | 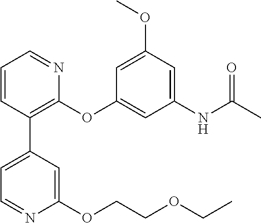 [SMILES: CCOCCOc1cc(-c2cccnc2Oc2cc(NC(C)=O)cc(OC)c2)ccn1] |
| 88 | 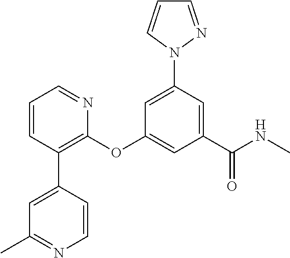 |
| 89 | 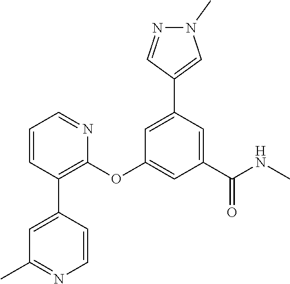 |
| 90 | 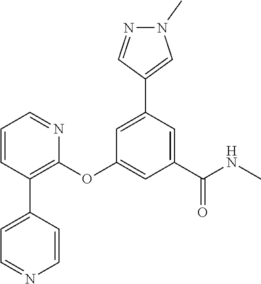 [SMILES: CNC(=O)c1cc(Oc2ncccc2-c2ccncc2)cc(-c2cnn(C)c2)c1] |
| 91 | 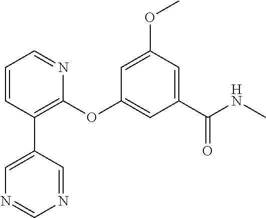 [SMILES: CNC(=O)c1cc(OC)cc(Oc2ncccc2-c2cncnc2)c1] |
| 92 | 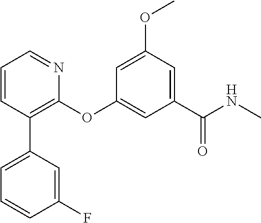 [SMILES: CNC(=O)c1cc(OC)cc(Oc2ncccc2-c2cccc(F)c2)c1] |
| 93 | 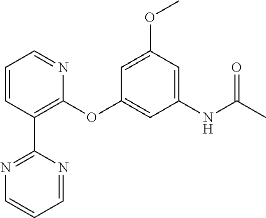 [SMILES: COc1cc(NC(C)=O)cc(Oc2ncccc2-c2ncccn2)c1] |
| 94 | 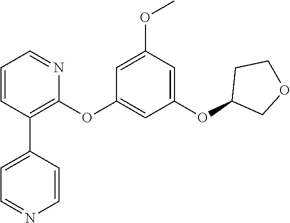 [SMILES: COc1cc(Oc2ncccc2-c2ccncc2)cc(O[C@H]2CCOC2)c1] |
| 95 | 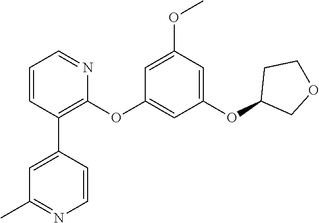 [SMILES: COc1cc(Oc2ncccc2-c2ccnc(C)c2)cc(O[C@H]2CCOC2)c1] |
| 96 | 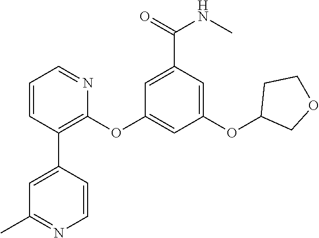 [SMILES: CNC(=O)c1cc(Oc2ncccc2-c2ccnc(C)c2)cc(OC2CCOC2)c1] |
| 97 | 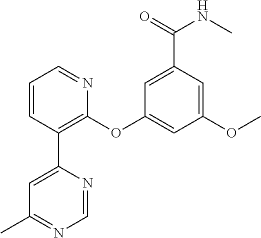 |
| 98 | 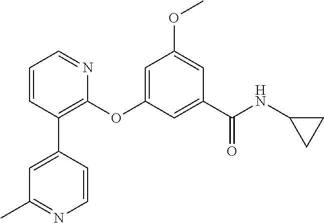 [SMILES: COc1cc(Oc2ncccc2-c2ccnc(C)c2)cc(C(=O)NC2CC2)c1] |
| 99 | 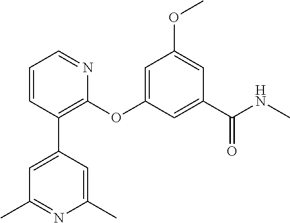 |
| 100 | 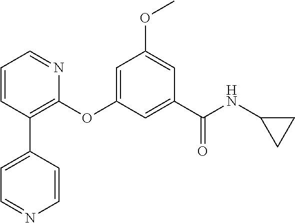 [SMILES: COc1cc(Oc2ncccc2-c2ccncc2)cc(C(=O)NC2CC2)c1] |
| 101 | 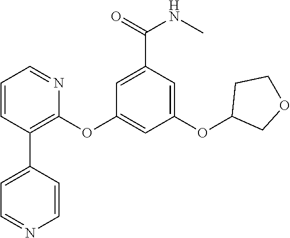 [SMILES: CNC(=O)c1cc(Oc2ncccc2-c2ccncc2)cc(OC2CCOC2)c1] |

or a pharmaceutically acceptable salt thereof.

16. A pharmaceutical composition comprising a compound of any of the previous claims and a pharmaceutically acceptable excipient.

17. A method of treat cancer comprising administering to a patient in need thereof the compound of any of claims 1-15 or the pharmaceutical composition of claim 16.

18. A compound of formula II: 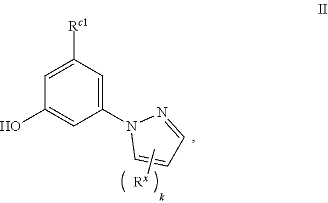
or a pharmaceutically acceptable salt,
wherein
R^{c1} is selected from the group consisting of C₁-C₆ haloalkyl, —OR¹, —C(O)OR¹, and optionally substituted 5-6 membered heteroaryl;
R^{x} is selected from the group consisting of halogen, —CN, —OH, —OR¹, —NH₂, —NR²R³, optionally substituted C₁-C₇ aliphatic, optionally substituted phenyl, —C(O)R¹, —C(O)OH, —C(O)OR¹, —C(O)NH₂, —C(O)NR²R³, optionally substituted 5-10 membered heteroaryl, and optionally substituted 5-10 membered heterocyclyl; and
k is 0, 1, 2, or 3.

19. The compound of claim 18, wherein the compound is of formula (II-a): 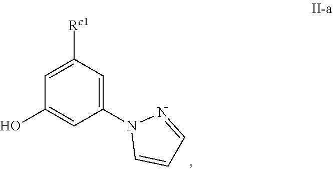
or a pharmaceutically acceptable salt thereof.

20. A compound represented by: 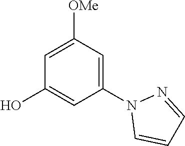 [SMILES: COc1cc(O)cc(-n2cccn2)c1]
or a pharmaceutically acceptable salt thereof.
